(19) United States
(12) Patent Application Publication
(10) Pub. No.: US 2023/0414583 A1
(43) Pub. Date: Dec. 28, 2023

(54) LYSERGIC ACID DERIVATIVES WITH MODIFIED LSD-LIKE ACTION

(71) Applicant: Mind Medicine, Inc., New York, NY (US)

(72) Inventors: Daniel TRACHSEL, Detligen (CH); Matthias Emanuel Liechti, Oberwil (CH); Felix Lustenberger, Meggen (CH)

(73) Assignee: Mind Medicine, Inc., New York, NY (US)

(21) Appl. No.: 18/337,253

(22) Filed: Jun. 19, 2023

Related U.S. Application Data
(60) Provisional application No. 63/353,673, filed on Jun. 20, 2022.

(51) Int. Cl. A61K 31/437; A61P 25/18

(52) U.S. Cl. CPC A61K 31/437; A61P 25/18

(57) ABSTRACT

A composition of a compound represented generically by FIG. 1A for use in substance-assisted therapy. A method of changing neurotransmission, by administering a pharmaceutically effective amount of a compound of FIG. 1A to a mammal, interacting with serotonin 5-HT2A receptors in the mammal, in particular also human beings, and inducing psychoactive effects. A method of treating an individual, by administering a pharmaceutically effective amount of a compound of FIG. 1A to the individual and treating the individual.

### BACKGROUND OF THE INVENTION

### 1. Technical Field

The present invention relates to both the substance definition and synthesis of lysergic acid derivatives with modified LSD-like action to be used in substance-assisted psychotherapy.

### 2. Background Art

Psychedelics are substances inducing unique subjective effects including dream-like alterations of consciousness, affective changes, enhanced introspective abilities, visual imagery, pseudo-hallucinations, synesthesia, mystical-type experiences, disembodiment, and ego-dissolution (Liechti, 2017; Passie, Halpern, Stichtenoth, Emrich, & Hintzen, 2008).

Psychedelics, mainly lysergic acid diethylamide (LSD) and psilocybin, are currently investigated as potential medications. First clinical trials indicate potential efficacy of LSD and psilocybin in addiction (Bogenschutz, 2013; Bogenschutz et al., 2015; Garcia-Romeu et al., 2019; Garcia-Romeu, Griffiths, & Johnson, 2014; Johnson, Garcia-Romeu, Cosimano, & Griffiths, 2014; Johnson, Garcia-Romeu, & Griffiths, 2016; Krebs & Johansen, 2012), anxiety associated with life-threatening illness (Gasser et al., 2014; Gasser, Kirchner, & Passie, 2015), depression (R. Carhart-Harris et al., 2021; R. L. Carhart-Harris, Bolstridge, et al., 2016; Davis et al., 2021; R. R. Griffiths et al., 2016; Roseman, Nutt, & Carhart-Harris, 2017; Ross et al., 2016), and anxiety (R. R. Griffiths et al., 2016; Grob et al., 2011; Ross et al., 2016). Several trials investigating therapeutic effects of LSD, psilocybin and other psychedelics are also ongoing. There is also evidence that the psychedelic brew Ayahuasca which contains the active psychedelic substance N,N-dimethyltryptamine (DMT) (Dominguez-Clave et al., 2016) may alleviate depression (Dos Santos et al., 2016; Palhano-Fontes et al., 2019; Sanches et al., 2016). In contrast, there are no comparable therapeutic studies or elaborated concepts on the use of psychedelic lysergic acid derivatives other than LSD to treat medical conditions.

Although no psychedelic is currently licensed for medical use, psilocybin and LSD are already used in special therapeutic-use programs (Schmid, Gasser, Oehen, & Liechti, 2021). LSD is a serotonergic psychedelic similar to psilocybin with comparable acute effects, although with significant longer duration of action (8-12 hours for LSD compared with 6 hours for psilocybin) (Becker et al., 2022; Holze et al., 2022; Holze, Vizeli, et al., 2021).

A potentially important disadvantage of LSD is its long duration of acute action resulting in the need for long days of supervising patients and related costs. On the other hand, LSD has advantages over psilocybin and other shorter-acting substances. In particular, there is a long history of use of LSD (Nichols, 2016) and substantial information on its safety pharmacology (Holze, Caluori, Vizeli, & Liechti, 2021; Nichols & Grob, 2018). The pharmacology of LSD is well studied (Holze, Caluori, et al., 2021; Holze et al., 2019; Holze, Vizeli, et al., 2021; Holze et al., 2020; Nichols, 2018b; Vizeli et al., 2021) and LSD is also among the most potent known psychedelic in vivo resulting in the need of only very low doses to produce the desired effect (Luethi & Liechti, 2018). Accordingly, it would be desirable to design an LSD analog with similar pharmacological properties in terms of potency, efficacy, and safety to LSD and with a similar or preferably different and faster metabolism and thus shorter duration of action than classic LSD. Novel lysergic acid derivatives can be equally suitable or superior to treat medical conditions. Specifically, existing psychedelic treatments such as LSD, psilocybin and DMT may not be suitable to be used in every patient considered for psychedelic-assisted therapy. Generally, the availability of several substances with different properties is important and the present lack thereof is a therapeutic problem which will further increase with more patients needing psychedelic-assisted therapy and an increase in demand for such treatment once the efficacy of first treatments is documented in large clinical studies. For example, some patients may react with strong adverse responses to existing therapies such as psilocybin presenting with untoward effects including headaches, nausea/vomiting, anxiety, cardiovascular stimulation, or marked dysphoria (Davis et al., 2021; R. R. Griffiths et al., 2016; Holze et al., 2022; Ross et al., 2016). On the other hand, the long duration of action of LSD may, in some cases, be a limited factor and the increased therapeutic session time may significantly contribute to the medical treatment costs. Further on, such long therapy sessions need tedious planning. Thus, novel compounds with psychedelic-like action are needed.

Structurally, LSD is an ergoline derivative, unlike psilocybin. Although they share some structural features such as the tryptamine core, the main pharmacophore of LSD remains significantly different to psychedelic tryptamines such as psilocybin and DMT, and their binding modes and overall pharmacological profiles are different (Cao et al., 2022; Rickli, Moning, Hoener, & Liechti, 2016; Wacker et al., 2017). Psychedelics from the ergoline, tryptamine and phenethylamine classes are all thought to induce their acute psychedelic effects primarily via their common stimulation of the 5-HT2A receptor. All serotonergic psychedelics including LSD, psilocybin, DMT, and mescaline are agonists at the 5-HT2A receptor (Rickli et al., 2016) and may therefore produce overall largely similar effects. However, there are differences in how the substances interact with the 5-HT2A receptor at the binding site and some compounds even bind to the receptor but do not produce subjective effects (Cao et al., 2022). Additionally, there are differences in the receptor activation profiles and in the subsequent signal transduction pathway activation patterns between the substances that may induce different subjective effects. Furthermore, LSD potently stimulates the 5-HT2A receptor but also 5-HT2B/C, 5-HT1 and D1-3 receptors (Rickli et al., 2016). Psilocin, i.e., the active metabolite present in the human body derived from the prodrug psilocybin, also stimulates the 5-HT2A receptor but additionally inhibits the 5-HT transporter (SERT) (Rickli et al., 2016). Mescaline binds in a similar, rather low concentration range to 5-HT2A, 5-HT2C, 5-HT1A and α2A receptors. In contrast to LSD, psilocybin and mescaline show no affinity for D2 receptors (Rickli et al., 2016). Taken together, LSD can have greater dopaminergic activity than psilocybin and mescaline, psilocybin can have additional action at the SERT. Mescaline and its derivatives do not interact with the SERT in contrast to psilocybin. Taken together, the pharmacological profiles of psychedelics may be different at the 5-HT2A receptor but clearly also regarding additional effects at other receptors which can then translate into different and even unique effect profiles for each substance.

In humans, subjective effects of psychoactive doses of LSD appear within 15-60 minutes, peak at 2-4 hours and dose-dependently last 8-12 hours. The plasma half-life is approximately 4 hours (Holze et al., 2019; Holze et al., 2022; Holze, Vizeli, et al., 2021). The long duration of action of LSD reflects the presence of LSD in plasma and is thus linked to the concentration-time curve in a specific subject and the plasma half-life (Holze et al., 2019). The same is true for psilocybin, where the presence of the active metabolite psilocin in its unconjugated form in plasma defines the duration of action of psilocybin in humans. The plasma half-life of unconjugated psilocin is on average 2 hours (Becker et al., 2022), consistent with the shorter duration of action of psilocybin compared with LSD. It can therefore be expected that a structurally related compound of LSD with a shorter plasma half-life would also have a similarly shorter duration of action.

The acute subjective effects of psychedelics are mostly positive in most humans (R. L. Carhart-Harris, Kaelen, et al., 2016; Dolder, Schmid, Mueller, Borgwardt, & Liechti, 2016; Dolder et al., 2017; Holze et al., 2019; Schmid et al., 2015). However, there are also negative subjective effects such as anxiety in many humans (Davis et al., 2021; R. R. Griffiths et al., 2016; Ross et al., 2016) likely depending on the dose used (Holze et al., 2022), personality traits (set), the setting (physical and social environment) and other factors (Studerus, Gamma, Kometer, & Vollenweider, 2012). The induction of an overall positive acute response to the psychedelic is critical because several studies showed that a more positive experience is predictive of a greater therapeutic long-term effect of the psychedelic (Garcia-Romeu et al., 2014; R. R. Griffiths et al., 2016; Ross et al., 2016). Even in healthy subjects, a more positive acute response to a psychedelic including LSD has been shown to be linked to more positive long-term effects on well-being (R. Griffiths, Richards, Johnson, McCann, & Jesse, 2008; Schmid & Liechti, 2018).

LSD has relevant acute side effects to different degrees depending on the subject treated and including increased blood pressure, nausea and vomiting, elevated body temperature and blood sugar, numbness, tremor, negative body sensations, and dysphoria (Holze, Caluori, et al., 2021). Such side effects of a substance are often linked to its interactions with pharmacological targets. For example, interactions with adrenergic receptors can result untoward clinical cardio-stimulant properties. Additionally, changes in the relative activation profile of serotonin 5-HT receptors and other targets change the quality of the psychoactive effects. Alterations in the binding potency, the binding mode, and the potency in activating the subsequent signaling pathways at 5-HT2A receptors as well as the molecule's lipophilicity can mostly determine the clinical dose to induce psychoactive effects. Alterations changing the metabolic stability of the compounds can also change the duration of action of the substance significantly.

New LSD-based derivatives, namely lysergic acid-based derivatives, are needed to provide substances with an improved effect profile such as, but not limited to, more positive effects, less adverse effects, different qualitative effects, and change of duration of acute effect.

### SUMMARY OF THE INVENTION

The present invention provides for a composition of a compound represented generically by FIG. 1A and named “Lysergic acid amides” for use in substance-assisted therapy.

As such, class 1 is a lysergic acid amide as represented in FIG. 2A to FIG. 2G, 3A to 3H, 4A to 4G, 5A to 5C and 6A to 6C, wherein R8′ is consisting of substituents shown in subclasses, named class 1a to 1n, whereby R8 is consisting of:
a) R8′,
b) any substituent of the subclasses 1a to 1n and R8′═ as defined in the specific class from 1a to 1l,
c) Hydrogen, C1-C5 alkyl, branched C1-C5 alkyl, C3-C5 cycloalkyl, C1-C5 alkylcycloalkyl, C2-C5 alkenyl, branched C3-C5 alkenyl, C2-C5 alkynyl, branched C4-C5 alkynyl, or
d) as specifically indicated in classes 1a to 1n;
   with that defined,
in class 1a, being a subclass of class 1, the substituent R8′ consists of an F1-F11 fluorine substituted C1-C5 alkyl or branched C3-C5 alkyl group, each optionally combined with D1-D10 deuteron, and/or hydroxy and/or carbonyl,
in class 1 b, being a subclass of class 1, the substituent R8′ consists of an F1-F13 fluorine substituted C3-C7 alkenyl group, optionally combined with D1-D12 deuteron, and/or nitrile, and/or hydroxy and/or carbonyl, whereby the double bond being isolated from the Nitrogen,
in class 1c, being a subclass of class 1, the substituent R8′ consists of an F1-F11 fluorine substituted C3-C6 cycloalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in class 1d, being a subclass of class 1, the substituent R8′ consists of an F1-F17 fluorine substituted C3-C6 cycloalkylalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in class 1e, being a subclass of class 1, the substituent R8′ consists of an F1-F11 fluorine substituted C3-C7 alkynyl group, optionally combined with D1-D12 deuteron, and/or nitrile, and/or hydroxy and/or carbonyl, with the triple bond isolated from the amide Nitrogen,
in class 1f, being a subclass of class 1, the substituent R8′ consists of an F0-F7 fluorine substituted C2-C4 alkenyl group attached to the Nitrogen with the unsaturated part, yielding enamides, optionally combined with D1-D7 deuteron, and/or nitrile, and/or hydroxy and/or carbonyl,
in class 1g, being a subclass of class 1, the substituent R8′ consists of an F1-F5 fluorine substituted C2-C4 alkylalkynyl group attached to the Nitrogen with the unsaturated part, yielding ynamides, optionally combined with D1-D4 deuteron, and/or nitrile, and/or hydroxy and/or carbonyl,
in class 1 h, being a subclass of class 1, the substituent R8′ consists of an F1-F13 fluorine substituted C1-3-O—C1-3 alkoxyalkyl group, optionally combined with D1-D12 deuteron, and/or nitrile, and/or hydroxy and/or carbonyl,
in class 1i, being a subclass of class 1, the substituent R8′ consists of an F0-F7 fluorine substituted C1-C3 alkoxy or C3-C4 cycloalkoxy group, each optionally combined with D1-D7 deuteron, and/or nitrile, and/or hydroxy and/or carbonyl,
in class 1j, being a subclass of class 1, the substituent R8′ consists of a nitrile attached to a C1-C3 alkyl group, optionally combined with F1-F7 fluorine, and/or D1-D7 deuteron, and/or hydroxy and/or carbonyl,
in class 1 k, being a subclass of class 1, the substituent R8 consists of any D1-D6 deuteron combined with F1-F6 fluorine containing C1-C3 alkyl group optionally combined with hydroxy and/or carbonyl, and R8′ consists of a Hydrogen, a C1-C6 alkyl or a C3-C5 cycloalkyl or a C4-C7 cycloalkylalkyl group, optionally combined with hydroxy and/or carbonyl,
in class 1l, being a subclass of class 1, the substituent R8 consists of a D1-D7 deuteron or an F1-F7 fluorine, or of any D1-D6 deuteron combined with F1-F6 fluorine containing C1-C3 alkyl group optionally combined with hydroxy and/or carbonyl, and R8′ consists of a C2-C8 alkenyl or a C2-C8 alkynyl group, optionally combined with nitrile, and/or hydroxy and/or carbonyl,
in class 1m, being a subclass of class 1, the substituent R8 and R8′ are connected to each other to build an azacycloalkane with the amide Nitrogen, and are consisting of a D1-D10 deuteron or an F1-F10 fluorine, or of any D1-D9 deuteron combined with F1-F9 fluorine containing C3-C6 alkylene group optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group,
in class 1n, being a subclass of class 1, the substituent R8 and R8′ are connected to each other to build an azacycloalkane with the amide Nitrogen and are consisting of a C3-C6 alkylene group having a nondeuterated or deuterated C1-C3 alkenyl and/or a nondeuterated or deuterated C2-C3 alkynyl group attached, the azacycloalkane forming alkylene group further and optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group.

The present invention provides for a composition of a compound represented generically by FIG. 1A and named “Lysergic acid amides” for use in substance-assisted therapy and is also represented by class 2, consisting of 6-substituted 6-Nor-lysergic acid diethylamides as represented in FIG. 4A to FIG. 5C, wherein R6 is consisting of substituents shown in subclasses, named class 2a to 2i, whereby R6 is consisting of as follows:
in class 2a, being a subclass of class 2, the substituent R6 consists of an F1-F11 fluorine substituted C1-C5 alkyl or branched C3-C5 alkyl group, each optionally combined with D1-D10 deuteron, and/or nitrile, and/or hydroxy and/or carbonyl,
in class 2b, being a subclass of class 2, the substituent R6 consists of an F1-F13 fluorine substituted C3-C7 alkenyl group, optionally combined with D1-D12 deuteron, and/or nitrile, and/or hydroxy and/or carbonyl, with the alkenyl double bond being isolated from Nitrogen,
in class 2c, being a subclass of class 2, the substituent R6 consists of an F1-F11 fluorine substituted C3-C7 alkynyl group with the triple bond isolated from N6 Nitrogen, optionally combined with D1-D10 deuteron, and/or nitrile, and/or hydroxy and/or carbonyl. In case the substituent R6 contains at least one nitrile, one hydroxy or one carbonyl group, R6 can also consist of a C3-C7 alkynyl group with the triple bond isolated from N6 Nitrogen, optionally combined with D1-D10 deuteron, and/or nitrile, and/or hydroxy and/or carbonyl,
in class 2d, being a subclass of class 2, the substituent R6 consists of a C3-C6 cycloalkyl group, optionally combined with F1-F11 fluorine, and/or D1-D11 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in class 2e, being a subclass of class 2, the substituent R6 consists of an F1-F17 fluorine substituted C4-C9 cycloalkylalkyl group, optionally combined with D1-D16 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl. In case the substituent R6 is not cyclopropylmethyl attached by the exocyclic methylene unit to the N6 Nitrogen of the ergoline structure, or it is cyclopropylmethyl attached by the exocyclic methylene unit to the N6 Nitrogen of the ergoline structure and contains at least one nitrile, one hydroxy or one carbonyl group, R6 can also consist of a C4-C9 cycloalkylalkyl group, optionally combined with D1-D17 deuteron, and/or nitrile, and/or hydroxy, and/or carbonyl, and/or deuterated and nondeuterated C1-C3 alkyl and/or deuterated and nondeuterated C1-C3 alkenyl and/or deuterated and nondeuterated C1-C3 alkynyl group,
in class 2f, being a subclass of class 2, the substituent R6 consists of an F0-F7 fluorine substituted C2-C4 alkenyl group attached to the Nitrogen with the unsaturated part, yielding enamines, optionally combined with D1-D7 deuteron, and/or nitrile, and/or hydroxy and/or carbonyl,
in class 2g, being a subclass of class 2, the substituent R6 consists of a C3-C6 oxacycloalkyl, a C3-C9 oxacycloalkylalkyl, a C3-C6 thiacycloalkyl or of a C3-C9 thiacycloalkylalkyl group, each optionally combined with F1-F19 fluorine, and/or D1-D19 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in class 2h, being a subclass of class 2, the substituent R6 consists of an F0-F11 fluorine substituted C1-C5 alkoxy or C3-C6 cycloalkoxy or C2-C6 alkenoxy group, each optionally combined with D1-D11 deuteron, and/or nitrile, and/or hydroxy and/or carbonyl,
in class 2i, being a subclass of class 2, the substituent R6 consists of an aryl, a heteroaryl, an arylmethyl or a heteroarylmethyl group, each optionally combined with F0-F7 fluorine, and/or D1-D7 deuteron, and/or one or more of the following substituents that themselves can optionally be fluorinated and/or deuterated: halogen, nitrile, nitro, hydroxy, carbonyl, C1-C4 alkoxy, C1-C4 alkyl, C1-C4 alkenyl, C1-C4 alkynyl, C3-C6 cycloalkyl, C3-C6 cycloalkoxy, C3-C6 alkenoxy, C3-C6 alkynoxy, methylenedioxy, C1-C4 alkylthio, C3-C6 alkenylthio, C3-C6 alkynylthio, C3-C6 cycloalkylthio. Further on, the R6 substituent, as defined for class 2i above, can further be annulated.

The present invention provides for a composition of a compound represented generically by FIG. 1A and named “Lysergic acid amides” for use in substance-assisted therapy and is also represented by class 3 (FIG. 6A), consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C).

The present invention provides for a composition of a compound represented generically by FIG. 1A and named “Lysergic acid amides” for use in substance-assisted therapy is also represented by class 4 (FIG. 6B), consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) any acyl; b) unsubstituted and substituted carbamoyl; c) amide-bound amino acid; d) alkyl, alkenyl or alkynyl; e) alkoxy, alkenoxy or alkynoxy; f) any of the substituents described under a) to e), substituted with one or more fluorine atoms; g) any of the substituents described under a) to e), substituted with one or more deuteron atoms; h) any of the substituents described under a) to e), substituted with one or more fluorine atoms and one or more deuteron atoms.

The present invention provides for a composition of a compound represented generically by FIG. 1A and named “Lysergic acid amides” for use in substance-assisted therapy is also represented by class 5 (FIG. 6C), consisting of a monodeuterated up to a fully deuterated ergoline core structure, and additionally consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) Hydrogen; b) any acyl; c) unsubstituted and substituted carbamoyl; d) amide-bound amino acid; e) alkyl, alkenyl or alkynyl; f) alkoxy, alkenoxy or alkynoxy; g) any of the substituents described under a) to f), substituted with one or more fluorine atoms; h) any of the substituents described under a) to f), substituted with one or more deuteron atoms; i) any of the substituents described under a) to f), substituted with one or more fluorine atoms and one or more deuteron atoms.

The present invention provides for a method of changing neurotransmission, by administering a pharmaceutically effective amount of a compound of FIG. 1A to a mammal, interacting with serotonin 5-HT2A receptors in the mammal, in particular also human beings, and inducing psychoactive effects.

The present invention provides for a method of treating an individual by administering a pharmaceutically effective amount of a compound of FIG. 1A to the individual and treating the individual.

### DESCRIPTION OF THE DRAWINGS

Other advantages of the present invention are readily appreciated as the same becomes better understood by reference to the following detailed description when considered in connection with the accompanying drawings wherein:

FIG. 1A shows the generic chemical structure of lysergic acid derivatives of the scope of invention, more specifically lysergic acid amides with substituents varied at the positions 1 (defined as R1), 6 (defined as R6) and at the amide part of the amide attached to position 8 (defined as R8 and R8′), FIG. 1B shows the ergoline core structure with its numbering, and FIG. 1C shows the chemical structure of LSD and its stereochemical designation;

FIG. 2A shows compounds defined as class 1, FIG. 2B shows compounds of subclass 1a, FIG. 2C shows compounds of subclass 1b, FIG. 2D shows compounds of subclass 1c, FIG. 2E shows compounds of subclass 1d, FIG. 2F shows compounds of subclass 1e, and FIG. 2G shows compounds of subclass 1f;

FIG. 3A shows compounds of subclass 1g, FIG. 3B shows compounds of subclass 1h, FIG. 3C shows compounds of subclass 1i, FIG. 3D shows compounds of subclass 1j, FIG. 3E shows compounds of subclass 1k, FIG. 3F shows compounds of subclass 11, FIG. 3G shows compounds of subclass 1m, and FIG. 3H shows compounds of subclass 1n;

FIG. 4A shows compounds defined as class 2, FIG. 4B shows compounds of subclass 2a, FIG. 4C shows compounds of subclass 2b, FIG. 4D shows compounds of subclass 2c, FIG. 4E shows compounds of subclass 2d, FIG. 4F shows compounds of subclass 2e, and FIG. 4G shows compounds of subclass 2f;

FIG. 5A shows compounds of subclass 2g, FIG. 5B shows compounds of subclass 2h, and FIG. 5C shows compounds of subclass 2j;

FIG. 6A shows compounds of class 3, FIG. 6B shows compounds of class 4, and FIG. 6C shows compounds of class 5;

FIGS. 7A-7N exhibits prepared examples of lysergic acid derivatives represented by FIG. 1, FIG. 7A shows compound 2a, FIG. 7B shows compound 2b, FIG. 7C shows compound 2c, FIG. 7D shows compound 2d, FIG. 7E shows compound 2e, FIG. 7F shows compound 2f, FIG. 7G shows compound 2g, FIG. 7H shows compound 2h, FIG. 7I shows compound 2i, FIG. 7J shows compound 2j, FIG. 7K shows compound 2k, FIG. 7L shows compound 2l, FIG. 7M shows compound 2m, and FIG. 7N shows compound 2n;

FIGS. 8A-8N exhibits prepared examples of lysergic acid derivatives represented by FIG. 1, FIG. 8A shows compound 12a, FIG. 8B shows compound 12b, FIG. 8C shows compound 12c, FIG. 8D shows compound 12d, FIG. 8E shows compound 12e, FIG. 8F shows compound 12f, FIG. 8G shows compound 12g, FIG. 8H shows compound 13, FIG. 8I shows compound 14a, FIG. 8J shows compound 14b, FIG. 8K shows compound 14c, FIG. 8L shows compound 16a, FIG. 8M shows compound 16b, and FIG. 8N shows compound 16c;

FIG. 9 summarily describes the synthetic route to the lysergic acid derivatives 2a-2k, 2l as well as 2m;

FIG. 10 summarily describes the synthetic route to the lysergic acid derivative 2n;

FIG. 11 summarily describes the synthetic route to the lysergic acid derivatives 12a to 12g, 13 as well as 14a to 14c;

FIG. 12 summarily describes the synthetic route to the lysergic acid derivatives 16a to 16c;

FIGS. 13A-13J are graphs showing the metabolism of 10 novel lysergic acid derivatives wherein FIG. 13A shows compound 2c, FIG. 13B shows compound 2d, FIG. 13C shows compound 2e, FIG. 13D shows compound 2h, FIG. 13E shows compound 2j, FIG. 13F shows compound 2l, FIG. 13G shows compound 2n, FIG. 13H shows compound 12c, FIG. 13I shows compound 16a, and FIG. 13J shows compound 16b; and

FIG. 14 describes the receptor interactions of the novel lysergic acid derivatives with LSD and psilocin as controls.

### DETAILED DESCRIPTION OF THE INVENTION

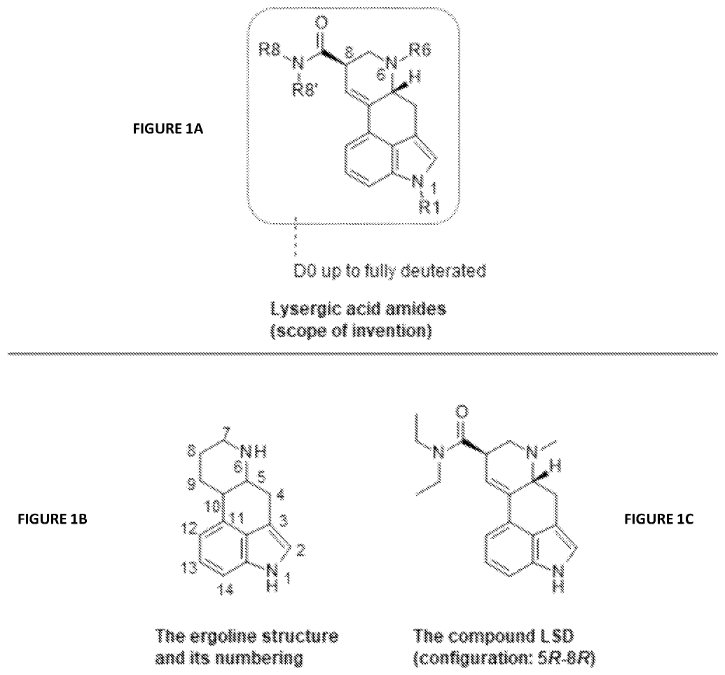

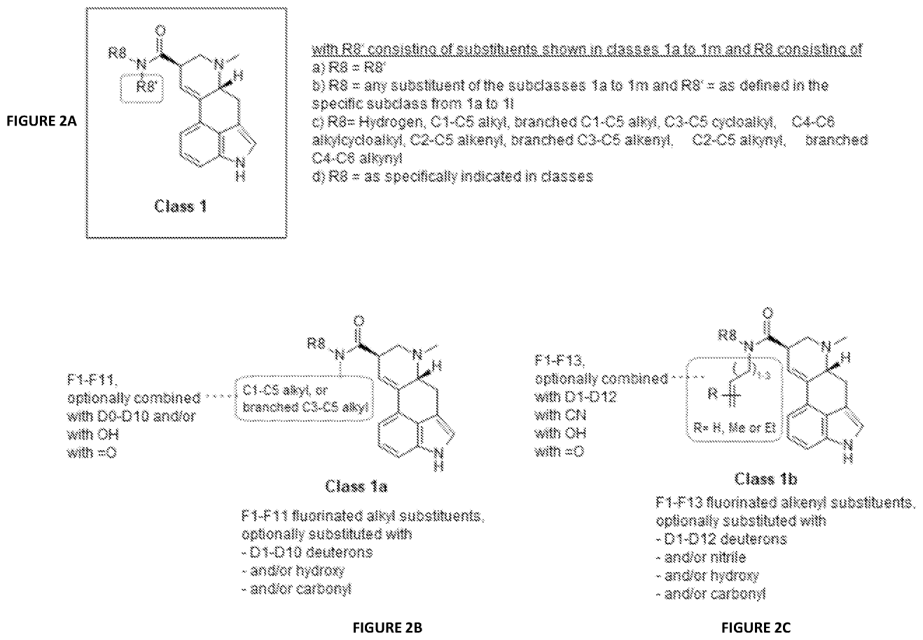

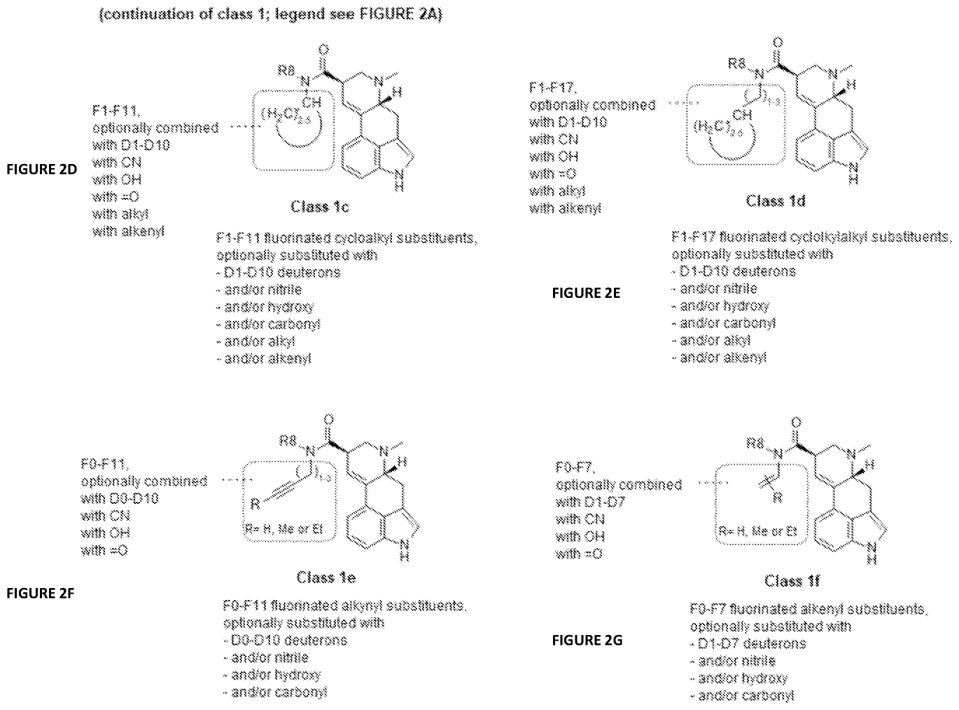

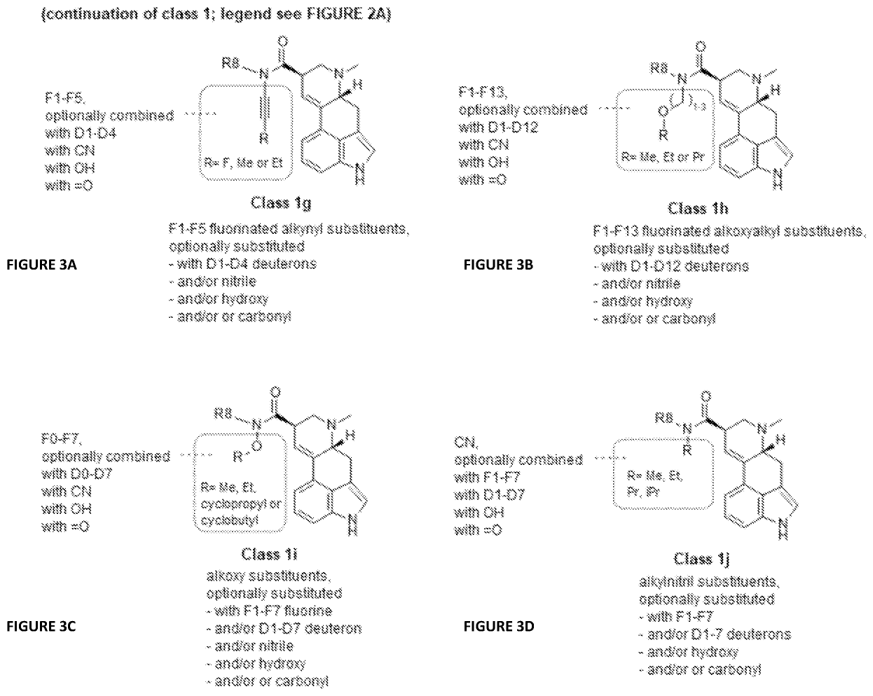

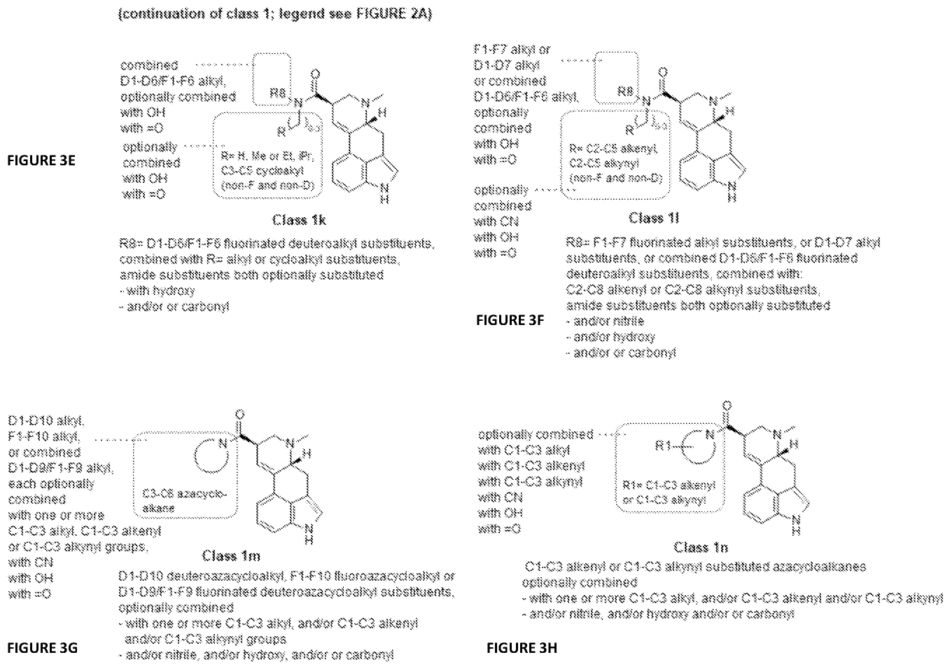

The present invention provides for a composition of a compound represented generically by FIG. 1A and named “Lysergic acid amides” for use in substance-assisted therapy. For a better understanding of the matter, such compounds are represented in FIG. 2A to FIG. 6C:

As such, class 1 is a lysergic acid amide as represented in FIG. 2A to FIG. 3H, wherein R8′ is consisting of substituents shown in subclasses, named class 1a to 1n, whereby R8 is consisting of:
a) R8′,
b) any substituent of the subclasses 1a to 1n and R8′═ as defined in the specific class from 1a to 1l,
c) Hydrogen, C1-C5 alkyl, branched C1-C5 alkyl, C3-C5 cycloalkyl, C1-C5 alkylcycloalkyl, C2-C5 alkenyl, branched C3-C5 alkenyl, C2-C5 alkynyl, branched C4-C5 alkynyl, or
d) as specifically indicated in classes 1a to 1n;
   with that defined,
in class 1a, being a subclass of class 1, the substituent R8′ consists of an F1-F11 fluorine substituted C1-C5 alkyl or branched C3-C5 alkyl group, each optionally combined with D1-D10 deuteron, and/or hydroxy and/or carbonyl,
in class 1b, being a subclass of class 1, the substituent R8′ consists of an F1-F13 fluorine substituted C3-C7 alkenyl group, optionally combined with D1-D12 deuteron, and/or nitrile, and/or hydroxy and/or carbonyl, whereby the double bond being isolated from the Nitrogen,
in class 1c, being a subclass of class 1, the substituent R8′ consists of an F1-F11 fluorine substituted C3-C6 cycloalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in class 1d, being a subclass of class 1, the substituent R8′ consists of an F1-F17 fluorine substituted C3-C6 cycloalkylalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in class 1e, being a subclass of class 1, the substituent R8′ consists of an F1-F11 fluorine substituted C3-C7 alkynyl group, optionally combined with D1-D12 deuteron, and/or nitrile, and/or hydroxy and/or carbonyl, with the triple bond isolated from the amide Nitrogen,
in class 1f, being a subclass of class 1, the substituent R8′ consists of an F0-F7 fluorine substituted C2-C4 alkenyl group attached to the Nitrogen with the unsaturated part, yielding enamides, optionally combined with D1-D7 deuteron, and/or nitrile, and/or hydroxy and/or carbonyl,
in class 1g, being a subclass of class 1, the substituent R8′ consists of an F1-F5 fluorine substituted C2-C4 alkylalkynyl group attached to the Nitrogen with the unsaturated part, yielding ynamides, optionally combined with D1-D4 deuteron, and/or nitrile, and/or hydroxy and/or carbonyl,
in class 1h, being a subclass of class 1, the substituent R8′ consists of an F1-F13 fluorine substituted C1-3-O—C1-3 alkoxyalkyl group, optionally combined with D1-D12 deuteron, and/or nitrile, and/or hydroxy and/or carbonyl,
in class 1i, being a subclass of class 1, the substituent R8′ consists of an F0-F7 fluorine substituted C1-C3 alkoxy or C3-C4 cycloalkoxy group, each optionally combined with D1-D7 deuteron, and/or nitrile, and/or hydroxy and/or carbonyl,
in class 1j, being a subclass of class 1, the substituent R8′ consists of a nitrile attached to a C1-C3 alkyl group, optionally combined with F1-F7 fluorine, and/or D1-D7 deuteron, and/or hydroxy and/or carbonyl,
in class 1 k, being a subclass of class 1, the substituent R8 consists of any D1-D6 deuteron combined with F1-F6 fluorine containing C1-C3 alkyl group optionally combined with hydroxy and/or carbonyl, and R8′ consists of a Hydrogen, a C1-C6 alkyl or a C3-C5 cycloalkyl or a C4-C7 cycloalkylalkyl group, optionally combined with hydroxy and/or carbonyl,
in class 11, being a subclass of class 1, the substituent R8 consists of a D1-D7 deuteron or an F1-F7 fluorine, or of any D1-D6 deuteron combined with F1-F6 fluorine containing C1-C3 alkyl group optionally combined with hydroxy and/or carbonyl, and R8′ consists of a C2-C8 alkenyl or a C2-C8 alkynyl group, optionally combined with nitrile, and/or hydroxy and/or carbonyl,
in class 1 m, being a subclass of class 1, the substituent R8 and R8′ are connected to each other to build an azacycloalkane with the amide Nitrogen, and are consisting of a D1-D10 deuteron or an F1-F10 fluorine, or of any D1-D9 deuteron combined with F1-F9 fluorine containing C3-C6 alkylene group optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group,
in class 1n, being a subclass of class 1, the substituent R8 and R8′ are connected to each other to build an azacycloalkane with the amide Nitrogen and are consisting of a C3-C6 alkylene group having a nondeuterated or deuterated C1-C3 alkenyl and/or a nondeuterated or deuterated C2-C3 alkynyl group attached, the azacycloalkane forming alkylene group further and optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group.

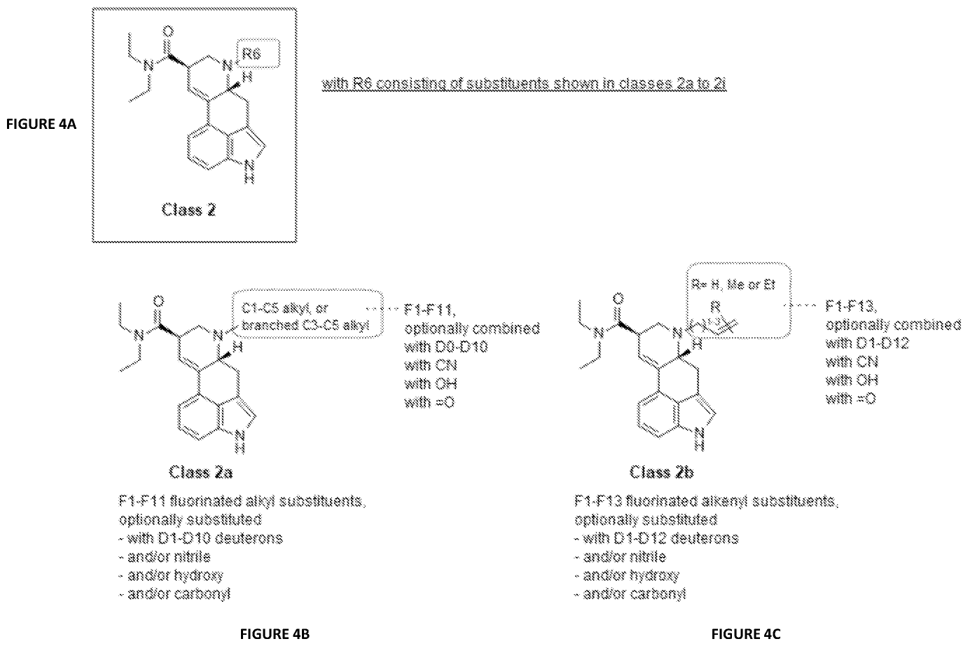

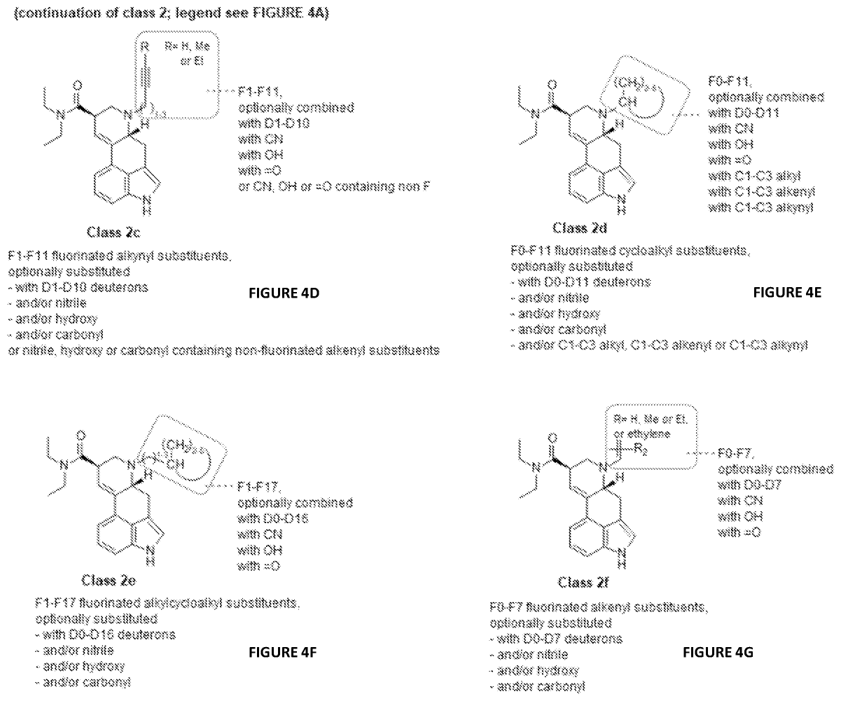

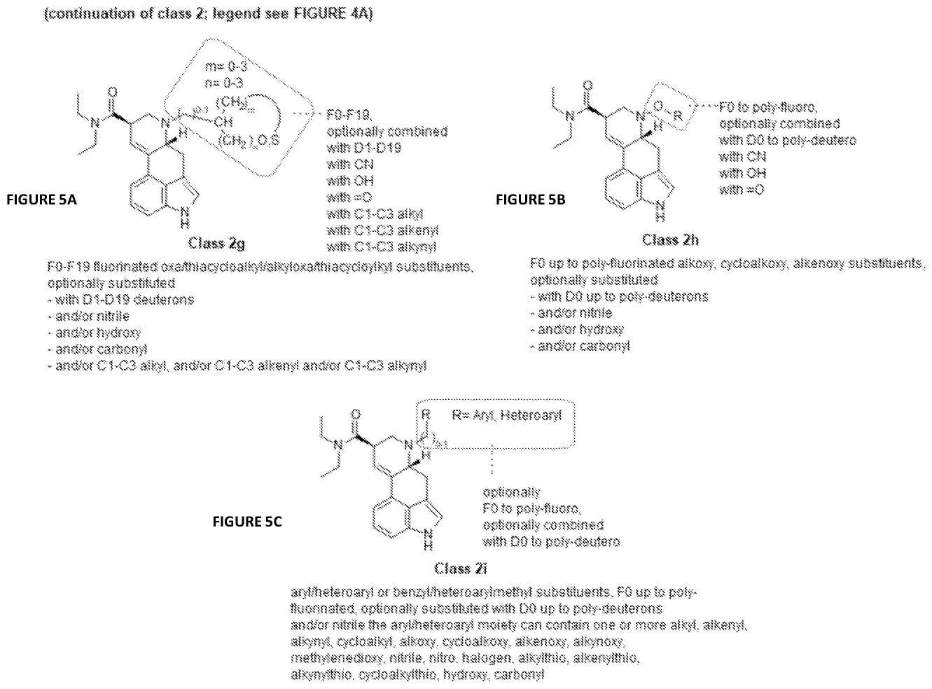

The present invention also provides for a composition of a compound represented generically by FIG. 1A and named “Lysergic acid amides” for use in substance-assisted therapy is also represented by class 2, consisting of 6-substituted 6-Nor-lysergic acid diethylamides as represented in FIG. 4A to FIG. 5C, wherein R6 is consisting of substituents shown in subclasses, named class 2a to 2i, whereby R6 is consisting of as follows:
in class 2a, being a subclass of class 2, the substituent R6 consists of an F1-F11 fluorine substituted C1-C5 alkyl or branched C3-C5 alkyl group, each optionally combined with D1-D10 deuteron, and/or nitrile, and/or hydroxy and/or carbonyl,
in class 2b, being a subclass of class 2, the substituent R6 consists of an F1-F13 fluorine substituted C3-C7 alkenyl group, optionally combined with D1-D12 deuteron, and/or nitrile, and/or hydroxy and/or carbonyl, with the alkenyl double bond being isolated from Nitrogen,
in class 2c, being a subclass of class 2, the substituent R6 consists of an F1-F11 fluorine substituted C3-C7 alkynyl group with the triple bond isolated from N6 Nitrogen, optionally combined with D1-D10 deuteron, and/or nitrile, and/or hydroxy and/or carbonyl. In case the substituent R6 contains at least one nitrile, one hydroxy or one carbonyl group, R6 can also consist of a C3-C7 alkynyl group with the triple bond isolated from N6 Nitrogen, optionally combined with D1-D10 deuteron, and/or nitrile, and/or hydroxy and/or carbonyl,
in class 2d, being a subclass of class 2, the substituent R6 consists of a C3-C6 cycloalkyl group, optionally combined with F1-F11 fluorine, and/or D1-D11 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in class 2e, being a subclass of class 2, the substituent R6 consists of an F1-F17 fluorine substituted C4-C9 cycloalkylalkyl group, optionally combined with D1-D16 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl. In case the substituent R6 is not cyclopropylmethyl attached by the exocyclic methylene unit to the N6 Nitrogen of the ergoline structure, or it is cyclopropylmethyl attached by the exocyclic methylene unit to the N6 Nitrogen of the ergoline structure and contains at least one nitrile, one hydroxy or one carbonyl group, R6 can also consist of a C4-C9 cycloalkylalkyl group, optionally combined with D1-D17 deuteron, and/or nitrile, and/or hydroxy, and/or carbonyl, and/or deuterated and nondeuterated C1-C3 alkyl and/or deuterated and nondeuterated C1-C3 alkenyl and/or deuterated and nondeuterated C1-C3 alkynyl group,
in class 2f, being a subclass of class 2, the substituent R6 consists of an F0-F7 fluorine substituted C2-C4 alkenyl group attached to the Nitrogen with the unsaturated part, yielding enamines, optionally combined with D1-D7 deuteron, and/or nitrile, and/or hydroxy and/or carbonyl,
in class 2g, being a subclass of class 2, the substituent R6 consists of a C3-C6 oxacycloalkyl, a C3-C9 oxacycloalkylalkyl, a C3-C6 thiacycloalkyl or of a C3-C9 thiacycloalkylalkyl group, each optionally combined with F1-F19 fluorine, and/or D1-D19 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in class 2h, being a subclass of class 2, the substituent R6 consists of an F0-F11 fluorine substituted C1-C5 alkoxy or C3-C6 cycloalkoxy or C2-C6 alkenoxy group, each optionally combined with D1-D11 deuteron, and/or nitrile, and/or hydroxy and/or carbonyl,
in class 2i, being a subclass of class 2, the substituent R6 consists of an aryl, a heteroaryl, an arylmethyl or a heteroarylmethyl group, each optionally combined with F0-F7 fluorine, and/or D1-D7 deuteron, and/or one or more of the following substituents that themselves can optionally be fluorinated and/or deuterated: halogen, nitrile, nitro, hydroxy, carbonyl, C1-C4 alkoxy, C1-C4 alkyl, C1-C4 alkenyl, C1-C4 alkynyl, C3-C6 cycloalkyl, C3-C6 cycloalkoxy, C3-C6 alkenoxy, C3-C6 alkynoxy, methylenedioxy, C1-C4 alkylthio, C3-C6 alkenylthio, C3-C6 alkynylthio, C3-C6 cycloalkylthio. Further on, the R6 substituent, as defined for class 2i above, can further be annulated.

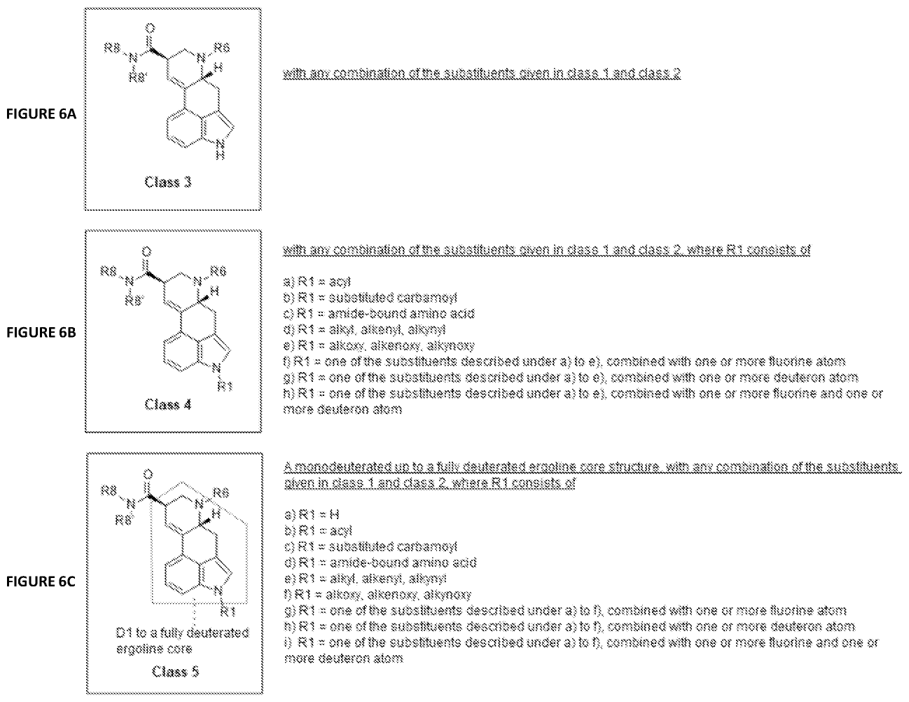

The present invention also provides for a composition of a compound represented generically by FIG. 1A and named “Lysergic acid amides” for use in substance-assisted therapy is also represented by class 3 (FIG. 6A), consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C).

The present invention also provides for a composition of a compound represented generically by FIG. 1A and named “Lysergic acid amides” for use in substance-assisted therapy is also represented by class 4 (FIG. 6B), consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) any acyl; b) unsubstituted and substituted carbamoyl; c) amide-bound amino acid; d) alkyl, alkenyl or alkynyl; e) alkoxy, alkenoxy or alkynoxy; f) any of the substituents described under a) to e), substituted with one or more fluorine atoms; g) any of the substituents described under a) to e), substituted with one or more deuteron atoms; h) any of the substituents described under a) to e), substituted with one or more fluorine atoms and one or more deuteron atoms.

The present invention provides for a composition of a compound represented generically by FIG. 1A and named “Lysergic acid amides” for use in substance-assisted therapy is also represented by class 5 (FIG. 6C), consisting of a monodeuterated up to a fully deuterated ergoline core structure, and additionally consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) Hydrogen; b) any acyl; c) unsubstituted and substituted carbamoyl; d) amide-bound amino acid; e) alkyl, alkenyl or alkynyl; f) alkoxy, alkenoxy or alkynoxy; g) any of the substituents described under a) to f), substituted with one or more fluorine atoms; h) any of the substituents described under a) to f), substituted with one or more deuteron atoms; i) any of the substituents described under a) to f), substituted with one or more fluorine atoms and one or more deuteron atoms.

The compounds represented by FIG. 1A are basic compounds which form acid addition salts with inorganic or organic acids. Therefore, they form pharmaceutically acceptable inorganic and organic salts with pharmacologically acceptable inorganic or organic acids. Acids to form such salts can be selected from inorganic acids such as hydrochloric acid, hydrobromic acid, hydroiodic acid, sulfuric acid, nitric acid, phosphoric acid, and the like, and organic acids, such as carbonic acid, p-toluenesulfonic acid, methanesulfonic acid, oxalic acid, succinic acid, citric acid, benzoic acid, and the like. Examples of such pharmaceutically acceptable salts thus are the sulfate, pyrosulfate, bisulfate, sulfite, bisulfite, phosphate, monohydrogen-phosphate, dihydrogenphosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methanesulfonate, propanesulfonate, mandelate and the like. Any hydrated form and any ratio of compound represented by FIG. 1A to pharmacologically acceptable inorganic or organic acids can be formed. Preferred pharmaceutically acceptable salts are those formed with tartaric acid and maleic acid.

Any of the pharmaceutically acceptable salt can also contain one or more deuteron or fluorine atoms and any stereoisomers are included.

The general chemical terms used for FIG. 1A to FIG. 12 have their usual meanings. Attachment of a generically named substituent to a molecule can be on any part of the substituent. For example, the term “alkyl” includes unbranched as well as branched alkyl groups, such as methyl, ethyl, n-propyl, isopropyl, n-butyl, isobutyl, sec-butyl, tert-butyl, and the like. For another example, the term “cycloalkyl” includes such groups as cyclopropyl, cyclobutyl, cyclopentyl, and the like. For yet another example, the term “alkylcycloalkyl” is used as “cycloalkylalkyl” and includes such groups as consisting of an alkyl as outlined before, coupled with a cycloalkyl as outlined before. Some examples include cyclopropylmethyl, cyclopropylethyl, cyclobutylmethyl, cyclopentylmethyl, (2-methylcyclopropyl)methyl, and the like. The term “oxacycloalkyl” includes such groups as oxetanes, tetrahydrofuranes, and the like. Further on, the term “alkenyl” includes unbranched as well as branched alkenyl groups, and includes such groups as vinyl (ethenyl), 1-propenyl, 2-propenyl, 1-butenyl, 2-butenyl, 3-butenyl and the like, with a configuration of cis, trans, E, or Z, in any combination or purity. Further on, the term “alkenyl” also includes alkylidenes such as methylidene, ethylidene and alike. Thus, the number one in “C₁-C₃ alkenyl” or in “C1-C3 alkenyl” can be used for methylidene, e.g., when a H₂C═ group is attached to a cycle. The term “alkylalkenyl” consists of any combination and branching of an alkenyl group with an alkyl group, with a configuration of cis, trans, E, or Z, in any combination or purity. Examples for such terms are 1-prop-2-enyl, 2-prop-1-enyl, 1-but-2-enyl, 1-but-3-enyl, 1-methyl-1-prop-2-enyl and the like. The term “alkynyl” includes unbranched as well as branched alkynyl groups, and includes groups such as ethynyl, 1-propyn-1-yl, 1-propyn-3-yl, 3-propynyl, 1-butynyl, 2-butynyl, 3-butynyl, phenylethynyl, and the like. The term “alkylalkynyl” consists of any combination and branching of an alkynyl group with an alkyl group. The term “alkylene” defines any unbranched or branched alkyl group serving as a connection between two molecular entities, substituents, or groups, or as an entity allowing to build a cycle together with the molecular entity, substituent, or group. For some examples, methylene, ethylene, propylene or methylpropylene are included, and as cycles, e.g., aziridines, azetidines, oxetanes are some examples. A phenyl group is defined as a substituent that can bear none or any numbers of substituents on the methylene or phenyl unit such as deuteron, fluorine, chlorine, bromine, iodine, methyl, ethyl, methoxy, methylthio, hydroxy, nitrile, methylenedioxy and the like. Such phenyl groups can further be annulated. An aryl group is defined as a substituent that contains one or more aromatic (annulated) homocycles, such as phenyl or naphthyl. A heteroaryl group is defined as any aromatic ring system containing a conjugate pi electron system causing aromaticity, such as thiophene, furane, pyrrole, selenophene, pyrazole, oxazole, thiazole, isoxazole, isothiazole, benzothiophene, benzofurane, pyridine, pyrimidine, pyrazine, and the like. Such heteroaryl groups can further be annulated. A benzyl substituent defines a phenylmethyl group that can bear none or any numbers of substituents on the methylene or phenyl unit such as deuteron, fluorine, chlorine, bromine, iodine, methyl, ethyl, methoxy, methylthio, hydroxy, nitrile, methylenedioxy and the like. Such benzyl groups can further be annulated. A Heteroarylmethyl consists of a heteroaryl group as defined before attached to a methylene unit and can bear none or any numbers of substituents on the methylene or phenyl unit such as deuteron, fluorine, chlorine, bromine, iodine, methyl, ethyl, methoxy, methylthio, hydroxy, nitrile, methylenedioxy and the like. Such heteroarylmethyl groups can further be annulated. The term “halogen” includes a fluorine, chlorine, bromine, and iodine substituent, and the number of halogens can be one to as much as chemically possible which corresponds to a completely halogenated substituent, also known under the term “polyhalogenated.” The term “deuterated” includes numbers of deuteron atoms that can be one to as much as chemically possible which corresponds to a completely deuterated substituent, also known under the term “polydeuterated”. Any ratios and additional stereoisomers caused by introduction of fluorine and/or deuteron atoms are included. Terms such as “F0 to F11 fluorinated” or “D0 to D5 deuterated” correspond to non-fluorinated up to undeca-fluorinated (eleven fluorine atoms), and nondeuterated to penta-deuterated (five deuterons), respectively. Similarly, this is also given with a term, as an example, “F₀-F₁₁ fluorine”, which means that the substituent can also contain zero fluorine and thus be non-fluorinated. The term “Aₘ-Aₙ”—m and n being a number from zero to 99 and indicating the amount of atoms A—is descriptive for the number of atoms A of a given group or substituent as a sum. An example for such terms is C₃-C₆ cycloalkylalkyl and means that it can include a cyclopropyl, a cyclobutyl, a cyclopropylmethyl or a cyclobutylethyl or any other cycloalkylalkyl group consisting of three to six carbons. In a similar way, as an example, “C₁₋₃—O—C₁₋₃” is descriptive for an alkoxyalkyl group consisting of an alkyl group with one to three carbons attached to an Oxygen attached itself to an alkyl group with one to three carbons. Such a representative alkoxyalkyl group can be, as examples, methoxymethyl, methoxyethyl, ethoxymethyl and alike. The counting number of atoms or substituents can either be shown with normal characters or with subscripted characters. Thus, as an example, “C₁₋₃—O—C₁₋₃” is being used equally to “C1-3-O—C1-3”. The term “lysergic acid derivatives” is used interchangeably with the term “lysergic acid amide,” “lysergic acid amides,” or “substituted lysergic acid amide,” or “lysergic acid derivatives” and alike, and all these terms are descriptive for the compounds of invention.

Those skilled in the art will appreciate that the compounds of the present invention have at least two chiral carbons, and may therefore exist as racemates, as individual enantiomers or diastereomers or epimers, and as mixtures of individual enantiomers or diastereomers or epimers in any ratio. Those skilled in the art will also appreciate that those compounds of the invention where R1, R6, R8 or R8′ in FIG. 1A consist of a chiral substituent, will bear an additional asymmetric center which create additional optical isomers as described above, and such compounds are within the scope of invention. While it is a preferred embodiment of the invention that the compounds of the invention exist are used as pure diastereomers with an absolute configuration of 5R,8R within the ergoline core structure, the present invention also contemplates the compounds of the invention existing in racemates or mixtures of individual enantiomeric or diastereomeric pure form.

Those skilled in the art will also appreciate that certain of the compounds of the present invention have at least one double bond leading, depending on the double-bond's substituents, to cis/trans or E/Z configurational isomerism. While it is a preferred embodiment of the invention that the compounds of the invention are used as pure configurational isomers, the present invention also contemplates the compounds of the invention existing in individual cis/trans or E/Z mixtures, respectively.

The individual enantiomers and diastereomers and epimers can be prepared by non-chiral or chiral chromatography of the racemic or enantiomeric or diastereomeric or epimeric mixtures of compounds represented by FIG. 1A, or fractional crystallization of salts thereof prepared from racemic or enantiomerically- or diastereomerically- or epimerically-enriched compound of invention and a chiral or non-chiral acid. Alternatively, the compounds of invention can be reacted with a chiral or non-chiral auxiliary and the enantiomers or diastereomers or epimers separated by chromatography or crystallization followed by removal of the chiral or non-chiral auxiliary to regenerate the compounds of invention. Furthermore, separation of enantiomers or diastereomers or epimers may be performed at any convenient point in the synthesis of the compounds of the invention. The compounds of the invention may also be prepared by application of chiral syntheses. The compound itself is a pharmacologically acceptable acid addition salt thereof.

The individual cis/trans or E/Z configurational isomers can be accessed by either selective synthesis or by separation techniques addressing the different physicochemical properties of the configurational isomers by applying techniques such as chromatography, crystallization, distillation, or extraction.

In patients that have adverse reactions to other psychedelics, lysergic acid derivatives can be useful as alternative treatments. In some patients, lysergic acid derivatives can also be useful because another experience than made with phenethylamines, psilocybin or LSD is necessary or because a patient is not suited for therapy with these existing approaches a priori. Thus, lysergic acid derivatives of FIG. 1A can serve as alternative treatment options with characteristics sufficiently similar to other psychedelics to be therapeutic but also sufficiently different to provide added benefits or avoid negative effects of other psychedelics.

Based on structural relations, the compounds of FIG. 1A described in the present invention are expected to have overall similar pharmacological properties as LSD.

This assumption is further emphasized by the handful of known and psycho-pharmacologically-described lysergic acid derivatives, compounds such as the N6-modified compounds ETH-LAD, PRO-LAD, ALL-LAD, the amide-modified compounds DAM-57, LPD-824 or LSM-775, as well as the N1-derivatized compounds ALD-52, OML-632 and MLD-41 which have shown psychoactive effects in human (Abramson, 1959; A. Shulgin & Shulgin, 1991)

The present invention provides compounds of FIG. 1A that are pharmacologically active and allow changing the neurotransmission and/or producing neurogenesis. More specifically, but not excluding, the compounds interact with serotonin (5-HT, 5-hydroxytryptamine) 5-HT2A and 5-HT2C receptors in mammals by administering to a mammal in need of such interaction a pharmaceutically effective amount of a compound of FIG. 1A.

Therefore, the present invention provides a method of changing neurotransmission, by administering a pharmaceutically effective amount of a compound of FIG. 1A to a mammal, increasing serotonin 5-HT2A receptor interaction in the mammal, and inducing psychoactive effects.

The present invention also provides generally for a method of treating an individual, by administering a pharmaceutically effective amount of a compound of FIG. 1A to the individual and treating the individual.

The condition or disease being treated can include, but is not limited to, anxiety disorders (including anxiety in advanced stage illness e.g. cancer, as well as generalized anxiety disorder), depression (including postpartum depression, major depressive disorder and treatment-resistant depression), headache disorder (including cluster headaches and migraine headache), obsessive compulsive disorder (OCD), personality disorders (including conduct disorder), stress disorders (including adjustment disorders and post-traumatic stress disorder), drug disorders (including alcohol dependence or withdrawal, nicotine dependence or withdrawal, opioid dependence or withdrawal, cocaine dependence or withdrawal, methamphetamine dependence or withdrawal), other addictions (including gambling disorder, eating disorder, and body dysmorphic disorder), pain, neurodegenerative disorders (such as dementia, Alzheimer's Disease, Parkinson's Disease), autism spectrum disorder, eating disorders, or neurological disorders (such as stroke).

The neuronal interaction of compounds represented in FIG. 1A can be used in mammals for substance-assisted psychotherapy where the compounds induce psychoactive effect to enhance psychotherapy. The preferred mammal is human.

The intensity and quality of the psychoactive effect including psychedelic or empathogenic (also called entactogenic or MDMA-like) effects (Holze et al., 2020), the quality of perceptual alterations such as imagery, fantasy and closed or open eyes visuals, and body sensation changes, the pharmacologically active doses, the duration of action may be different or similar to that of LSD.

LSD and some of its modified derivatives are known to interact with serotonin 5-HT2A, 5-HT2C, 5-HT1A, as well as with dopamine receptors (Nichols, Frescas, Marona-Lewicka, & Kurrasch-Orbaugh, 2002; Rickli et al., 2016; Watts et al., 1995).

LSD and some of its modified derivatives are also known to substitute for LSD in a two-lever drug discrimination assay (Nichols et al., 2002).

Among the known lysergic acid derivatives with psychoactive properties there have been investigated mainly three structural regions of the original LSD molecule.

One structural feature investigated earlier is substitution of the N1 in the LSD molecule, leading to N-acyl (e.g., N-acetyl, N-propionyl, N-butyryl), N-alkyl (e.g., N-methyl) or N-methoxy substituted LSD derivatives (Abramson, 1959; Halberstadt et al., 2020).

Some of these substituents are prone to fast metabolism and it was found that the compounds behave as prodrugs and only after N1-deprotection the compounds are active at the target receptors and psychoactive.

The second structural feature of the original LSD molecule modified earlier to gain psychoactive compounds is the N6-substituent. As such, the N6-methyl group was replaced by alkyl, allyl, propargyl, phenethyl, branched alkyl, alkylcycloalkyl (Hoffman & Nichols, 1985; Huang, Marona-Lewicka, Pfaff, & Nichols, 1994; Nichols, 2018a; Nichols et al., 2002; Nichols, Monte, Huang, & Marona-Lewicka, 1996; Oberlender, Pfaff, Johnson, Huang, & Nichols, 1992; Pfaff, Huang, Marona-Lewicka, Oberlender, & Nichols, 1994; A. Shulgin & Shulgin, 1991) and WO2021019023A1, WO2021175816A1. A few of the compounds were investigated in human and were just touched upon being psychoactive, and only three such compounds were described, at least in anecdotal reports, to be psychedelic (A. Shulgin & Shulgin, 1991).

The third structural feature of the original LSD molecule modified—or, mentioned only theoretically—to get potentially psychoactive compounds were the substituents of the amide group attached to the C8 atom of LSD. As such, N-monoalkyl, branched N-monoalkyl, symmetrical and unsymmetrical N,N-dialkyl, N-alkyl-N-alkenyl, N,N-dialkenyl, N,N-dialkynyl, N-ethyl-N-(2,2,2-trifluoroethyl), N-ethyl-N-(2-methoxyethyl), N-cycloalkyl, N-alkyl-N-cycloalkyl, N-alkyl-N-cycloalkyl or N-oxacycloalkyl derivatives have been described or mentioned as a theoretical idea. (Brandt et al., 2020; Huang et al., 1994; Nichols, 2018a; Nichols et al., 2002; Nichols et al., 1996; Oberlender et al., 1992; Pfaff et al., 1994; Watts et al., 1995) and WO2021019023A1, WO2021175816A1.

However, some of these compounds have only described in theory and have never been prepared chemically and investigated biologically, or even psycho-pharmacologically. Thus, it remains unclear to what extent some of these compounds show psycho activity in general or, more specifically, psychedelic properties.

When it comes to a combination of the aforementioned structural modification of the original LSD molecule, namely on N1, N6 and amide function attached to C8, hardly any compounds are known, one of the few exceptions being the N1-propionyl version of ETH-LAD (Brandt et al., 2017). Other compounds have only been described theoretically in, e.g., WO2021019023A1, WO2021175816A1 but their preparation and chemical characterization was never described.

One of the main reason for this lies in the unremittingly seek for N,N-diethylamide substituted derivatives of lysergic acid, driven by the findings that as soon as even one of the ethyl groups of the original LSD molecule is structurally altered, e.g., to a methyl, propyl or isopropyl group, the subsequent compound significantly loses its potency of psychoactive doses. Only very few structural modifications of the N,N-diethylamide moiety are allowed to retain at least some of the psychoactive properties, whereby the nature of the retained psycho activity remains elusive and has not been described in detail.

Another reason for the extremely limited number of chemically prepared and biologically investigated samples of lysergic acid derivatives bearing an amide different from N,N-diethylamide combined with a N6-substituent different than N-methyl lies in the laborious access of these compounds. By knowing from existing, previously described structure-activity relationship, that when not using N,N-diethylamide as the amide substituent, there seemed to be little interest in doing synthetic effort for getting additional examples of compounds that bear this extremely rare combinations of pharmacophores.

One reason the N6-substituent consists mostly of a methyl group in the aforementioned compounds lies in the use of lysergic acid as starting material; this acid is found chemically bound, as a chemical substructure, in nature mainly in ergot fungi, from which the compound ergotamine can be isolated. A hydrolysis of ergotamine and subsequent purification delivers pure lysergic acid, a compound otherwise chemically accessible only with extreme efforts.

Another reason that contributes to the broadly retained “original” 6-methyl substituent can be the synthetic conditions that were used in past to remove this methyl group. By this, the classical Von-Braun reaction applies cyanogen bromide in boiling tetrachloromethane, both highly problematic compounds to handle.

Taken together, virtually all lysergic acid derivatives with known psychoactive properties contain either the N,N-diethylamide pharmacophore with the N6-substituent varied, or the N6-substituent is retained as N6-methyl and the amide part is varied.

The nature of the psychoactive properties of the hitherto known psychoactive lysergic acid derivatives is often not described in detail and it remains unclear whether they behave as stimulants, as entactogens or as psychedelics (Holze et al., 2020) or a combination thereof.

In case of psychedelic properties of hitherto known lysergic acid derivatives, the nature of psychopharmacology (e.g., subjective effects profile compared with other substance) has only been described in detail and clinically for LSD (Holze et al., 2022; Holze, Vizeli, et al., 2021; Holze et al., 2020). Thus, it remains unclear whether any formerly described lysergic acid derivative would be suitable in the scope of invention mentioned herein at all.

Some of the invented lysergic acid compounds represented by FIG. 1A show in vitro pharmacological activity at the relevant target (5-HT2A receptor; Liechti et al. data on file) in comparison to LSD and indicating psychedelic action.

Introduction of one fluorine in one of the N-ethyl amide substituents is expected to retain psychedelic properties of the LSD molecule (for example in compound TRALA-04).

Introduction of one fluorine in the N6-substituent of ET-LAD retains psychedelic properties of the LSD molecule (as in compound TRALA-15).

As a conclusion, it is highly likely that a combination of these structural features also leads to a lysergic acid derivative with psychedelic properties.

The few hitherto known psychedelic active lysergic acid derivatives all show a similar duration of action with only little differences, mainly in the range of 8-12 hours. Duration of action is dependent mostly on the elimination half-life (Holze et al., 2019; Holze et al., 2022; Holze, Vizeli, et al., 2021), although also receptor occupation and kinetics may play a role (Wacker et al., 2017).

Metabolism of the original LSD molecule has been investigated in human biological fluids (Canezin et al., 2001). Main metabolic attacks were identified to occur in a) the diethylamide part to either N-monodeethlyation or monohydroxylation on one of the ethyl groups, b) N6-demethylation to form 6-Nor-LSD, c) oxidation/hydroxylation in the indole moiety. Only recently, it was shown by Vizeli et al. that a genetic influence of CYP2D6 on pharmacokinetics and acute subjective effects of LSD occurs in healthy subjects (Vizeli et al., 2021). The main metabolite of LSD in humans is 2-oxo-3-hydroxy-LSD (Luethi, Hoener, Krahenbuhl, Liechti, & Duthaler, 2019), therefore the main metabolic attack occurs at the indole part of LSD.

Due to the rather long and in some cases unfavorably long duration of psychedelic action of the original LSD molecule the inventors chose, as an option and not limiting, an “anti-stability approach” that is opposite to the usual way of optimizing pharmacologically active molecules. In classical medicinal chemistry, one goal is to keep or increase biological activities while/whereby also increasing metabolic stability. For some compounds of invention and represented by FIG. 1A, the inventors introduced atoms or functional groups that may enhance liability to metabolism. With this metabolism-enhancing strategy, a destabilization of substituents and re-stabilization by adding specific atoms or groups can allow a fine-tuning of metabolism while retaining psychedelic properties of the original LSD molecule.

Since it is also within the scope of invention to access psychedelic lysergic acid derivatives with shorter duration of action in comparison to the original LSD molecule, with structures represented by FIG. 1A two regions were identified to be modified but it is not limited to them.

Substitution on N1 of the original LSD molecule need, if psychedelic properties are key properties, a metabolically liable group attached to this position that releases the parent compound upon metabolism since, according to the existing SAR, nearly no substituents seem to be tolerated in this position to get agonistic serotonin 5-HT2A receptor ligands (Halberstadt et al., 2020), the primary site responsible for psychedelic properties of lysergic acid derivatives. Thus, LSD or similar compounds substituted on N1 mostly serve as prodrugs only and liberate the parent compound. Accordingly, duration of action is either be unchanged or rather be prolonged than shortened. One of the few exceptions where N1-substituents can lead to metabolically unchanged active compounds is the N1-substituted 1-methyl-LSD (Abramson, 1959), but at this point this remains unclear, and the compound showed significantly lower potency in human. Nevertheless, N1-substituents are within the scope of invention since they can contribute to modify duration and nature of action of the lysergic acid derivatives represented by FIG. 1A by co-influencing physico-chemical, and potentially absorption, distribution, metabolism, and elimination (ADME) properties.

The aforementioned “anti-stability approach” was applied on the N6 nitrogen of some of the lysergic acid derivatives represented by FIG. 1A. A metabolism can lead to, but is not limited to, polar conjugates and/or decompositions up to a completely unsubstituted N6 nitrogen lysergic acid derivatives structure (i.e., a secondary amine). It is known that N6-demethylation of LSD (i.e. 6-Nor-LSD) leads to a change of in vivo pharmacological properties (Fehr, Stadler, & Hofmann, 1970) and CH535236A as well as to a loss of its 5-HT2A binding affinity (factor 30) and the ability to substitute in LSD-trained rats, even at 20× of the full active dose of LSD (Hoffmann, 1987). However, in more recent binding assays, high-potency binding to 5-HT2A but not 5-HT2C receptors is retained with Nor-LSD (Luethi et al., 2019) indicating that this molecule and the presently described analogs and prodrugs can have relevant psychoactive properties. Thus, the presently designed and synthesized compounds need to be further investigated to clarify their psychoactive properties. The formation of other metabolites than N6-deprotection on compounds represented by FIG. 1A also can lead to inactivation of the parent compound and thus influence duration of action.

Further on, the aforementioned “anti-stability approach” was applied on the amide group of some of the lysergic acid derivatives represented by FIG. 1A. As such, the amide substituents within the scope of invention can undergo or can lead to a different or faster metabolism than the diethylamide of the original LSD molecule leading to inactivation of the psychoactive properties of the parent compounds and thus influence the duration of action.

A different metabolism provoked by the N1, N6 or amide substituents different to that of the original LSD molecule may also take place on any part of the chemical structure of the invented compounds represented by FIG. 1A and is not limited to a varied substituent itself.

In no way is the “anti-stability approach” limiting the scope of invention, and for compounds represented by FIG. 1A there is not necessarily required a metabolism that is different, faster, slower or similar to that of the original LSD molecule since other mechanisms onto the invented compounds can equally lead to different or similar duration of action, intensity and quality of the psychoactive effect including psychedelic or empathogenic effects, the quality of perceptual alterations such as imagery, fantasy and closed or open eyes visuals, and body sensation changes and/or the pharmacologically active doses.

The aforementioned modifications can take place on either N6 or on the amide part or in any combination thereof.

Not only receptor interactions, receptor profiles, subsequent signal transduction cascades, receptor heterodimerization, overall psychological and psychedelic effects can change by structural modifications represented in FIG. 1 but also the metabolism can be modified significantly by making, as an example, but not limited to, on N1, N6 or on the amide nitrogen, a potentially rather labile N-alkoxy compound, geminal amino ether compound, geminal amido ether (N-alkoxymethyl derivatives of amides) compound, vinyl or ethynyl compound (i.e. enamides, ynamides, enamines, ynamines), all more or less prone to metabolism by introducing, independently and in any combination, none or one or several alkyl groups, fluorine atoms or deuterium atoms to these functional groups in either vinyl, allyl or gamma positions, or in ethynyl or propargyl positions as aforementioned. Additionally, nitrile groups can also be introduced in any of these positions on the aforementioned substituents, and alkylnitriles or alkenylnitriles or alkynylnitriles, each with no or any fluorine and/or deuteron substituent are also an option. Further on, independently fluorinated, deuterated, or nitril-substituted N-allyl or N-propargyl groups are also within the scope of invention. Thus, the invention allows also for the synthesis of psychedelic compounds with a relatively shorter duration of action compared to more metabolically stable and longer-acting compounds.

Any of the aforementioned substituent attached to N1, N6 or to the amide can additionally also be combined with a substituent attached to the amide or to N6 or N1 consisting of an alkyl, alkenyl or alkynyl, cycloalkyl, alkylcycloalkyl, benzyl, heteroarylmethyl, each containing none, one or several fluorine, deuterium atoms or nitrile groups.

In another embodiment, any of the aforementioned structural modifications can be combined with one or several fluorine and/or deuterium atoms in any combination on the whole lysergic acid core, namely the ergoline core structure. As such to mention, but not limiting in any way, is the introduction of a deuteron at C8 of the ergoline structure to stabilize lysergic acid derivatives represented by FIG. 1A from epimerization. Epimerization can take place in dependance of the chemical/biological environment and is driven by factors such as pH value and, possibly, also by enzymatic activities. It is known that only the 5R,8R epimer of LSD is psychoactive, and its 5R,8S epimer, also known as iso-LSD is inactive up to several milligrams. The two remaining epimers (5S,8R and 5S,8S configurations) are psycho inactive as well.

The chemical stability of aforementioned functional groups such as enamides, ynamides, alkoxyamides (also known as Weinreb amides), geminal N-amidoethers, enamines, ynamines, alkoxyamines or geminal aminoethers towards acidic, basic or any other chemical conditions is dependent on factors such as pH, solvent medium, temperature, surface, nucleophilicity or electrophilicity of reaction partners or on gas containment of the environment. Metabolic stability in a biological environment such as a human body is additionally driven by factors such absorption rate, exposure to enzymes, enzyme activity, genetic polymorphism, retention time in a body medium such as gastrointestinal tract, rate of body distribution or transportation times. All these aspects can be influenced by the changes introduced to the compounds and result in the desired effects and effect-durations in humans.

The stability of a functional group, a substituent or, generally spoken, a molecule, towards aforementioned factors can significantly be influenced and modified by specific incorporation of stabilizing or destabilizing atoms or atom groups. Furthermore, the overall metabolic stability of a compound is also driven by properties such as the overall lipophilicity, three-dimensional structure, dissociation constants, solubility, steric accessibilities and steric bulkiness and other characteristics.

Fluorine is a strong electron-withdrawing atom and its incorporation to a substituent can significantly reduce the electron richness. Further on, it modifies dipole moment, dissociation constants of acidic and basic groups, the lipophilicity, pH value, and, to a certain extent, also steric properties of a fluorine-containing molecule are influenced. Thus, fluorine can change physicochemical properties and incorporation into a molecule can have a dramatic influence on interaction with biological targets, on chemical/metabolic stabilities and on metabolic pathways. Fluorine atoms incorporated to a molecule further allow so-called multipolar interactions with partially charged functional groups. This makes fluorine as an excellent tool for medicinal chemistry.

Deuteron is a stable isotope of hydrogen. Due to its slightly different metric, incorporated into a molecule it can influence physicochemical properties. With this, kinetic isotope effects, inverse kinetic isotope effects and also steric isotope effects can be observed. Chemical bonds involving deuterium are stronger and of different length compared to protium (hydrogen), which make such compounds significantly different in biological reactions. Thus, incorporation of a deuteron into a molecule can greatly influence its biological stabilities.

Consequently, both fluorine and deuteron can be used to replace or to be added to a substituent in order to modify the overall stability and biological properties of the compounds invented and represented by FIG. 1A and class 1 to class 5 as represented in FIGS. 2A to 6C. Replacement can be done with one or more fluorine atoms or with one or more deuteron atoms or in any combination of fluorine and deuteron atoms.

In analogy to these medicinal chemistry concepts, the biological properties of the invented compounds (ADME, target selectivity and target interaction, the mode of action, duration of action, the psychodynamic processes, and the qualitative perceptions, e.g., in terms of psychedelic or empathogenic intensity in comparison to the original LSD molecule) can not only be influenced by the aforementioned application of fluorine or deuteron to the functional groups such as enamides, ynamides, alkoxyamides, geminal N-amidoethers, enamines, ynamines, alkoxyamines or geminal aminoethers but also by introducing them to simpler substituents such as alkyl, alkenyl or alkynyl, cycloalkyl, oxacycloalkyl, alkylcycloalkyl, alkyloxacycloalkyl, benzyl or heteroarylmethyl substituents attached to N1, N6 or to the amide function attached to C8 of the ergoline core structure (FIG. 1A). Other atoms or atom groups can be used in a similar way to all these substituents, as outlined in FIGS. 2A-6C.

From older structure-activity relationships (Brandt et al., 2020; Huang et al., 1994; Nichols, 2018a; Nichols et al., 2002; Nichols et al., 1996; Oberlender et al., 1992; Pfaff et al., 1994; Watts et al., 1995) it is known, that the amide function of lysergic acid amides does not tolerate larger groups than N,N-diethyl substituents without losing biologic activity such as receptor affinities at receptors such as the 5-HT2A receptor relevant for human psychoactive effects. Surpassing its size or using a smaller group such as a methyl group has led to a quite impressive loss of binding properties on the 5-HT2A receptor as well as on human potency. When modifying the N6-substituent, examples found in literature (Hoffman & Nichols, 1985; Huang et al., 1994; Nichols, 2018a; Nichols et al., 2002; Nichols et al., 1996; Oberlender et al., 1992; Pfaff et al., 1994; A. Shulgin & Shulgin, 1991) have shown that expansion of the N6-methyl group up to a certain degree is tolerated for retaining in vivo potency, as shown, e.g., in drug discrimination studies or with anecdotal reports based on administration to humans. However, the inventors are not solely intending to access compounds with in vitro or in vivo potencies similar or higher than the prototypical LSD per se. In fact, a favorable overall profile may become more relevant, and lower potencies do in no way limit the use of such compounds.

LSD is normally used by oral administration. Buccal or nasal resorption as well as intravenous or intramuscular application has also been used. While the compounds represented by FIG. 1A can be used orally (i.e., resorption in the gastrointestinal tract), for certain compounds of the scope of invention the preferred route of administration can be buccal, nasal, intestinal, intravenous, or intramuscular application. These routes of administration can result in faster onset of the drug effect in addition to the modified duration of action of the compound itself. There are also important differences that can be expected for some of the compounds between oral and parenteral administration. For example, a compound can be destroyed or turned into LSD after oral administration by gastric or enteral fluids or enzymes whereas it can be metabolized differently when used parenterally. Thus, any change in the structure can differently affect oral versus parenteral administration.

While all the lysergic acid derivatives represented in FIG. 1A are useful in optimizing the overall biological and clinical effect profile of psychedelics, certain classes of the compounds are preferred, such as wherein the compound is a free base, a salt, a hydrochloride salt, a racemate where applicable, a single enantiomer, a single diastereomer, a singe epimer, or a mixture of enantiomers or diastereomers or epimers in any ratio, or an individual of a cis/trans or E/Z configurational isomer, or a mixture of these configurational isomers in any ratio. It will be understood that these classes can be combined to form additional preferred classes.

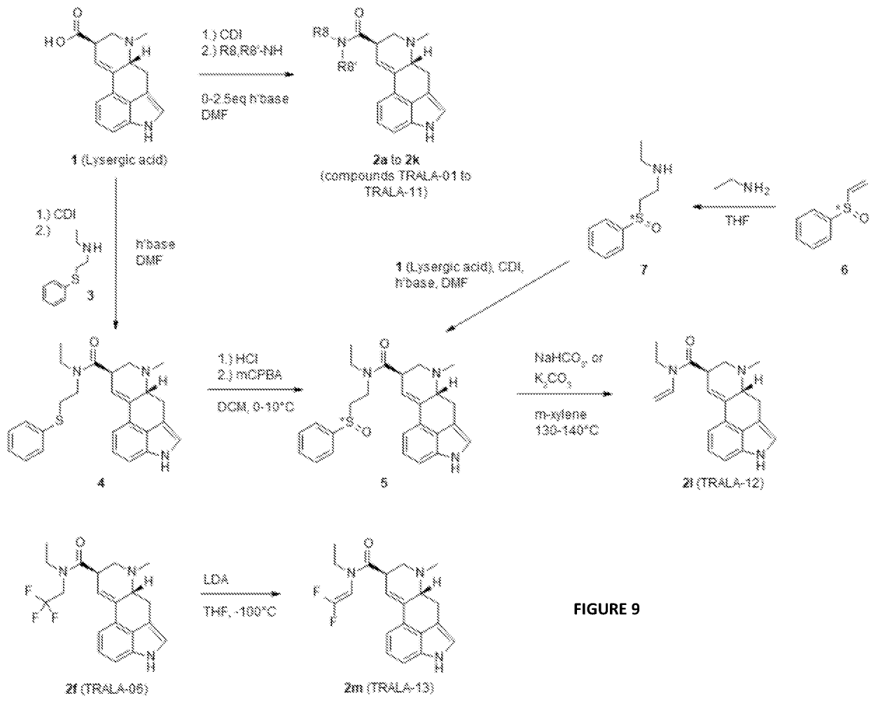

The synthetic access to the compounds of invention is shown in FIG. 9 to FIG. 12 and is given in detail in the section “Preparation of the compounds”.

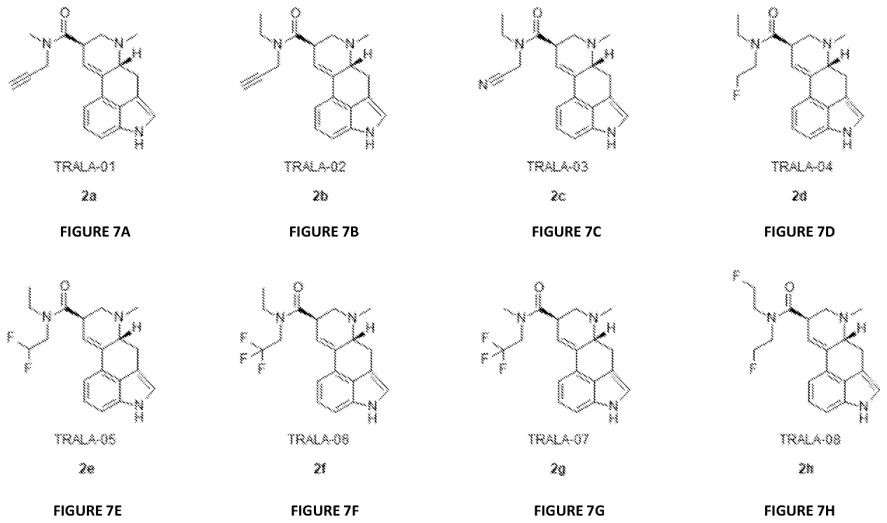

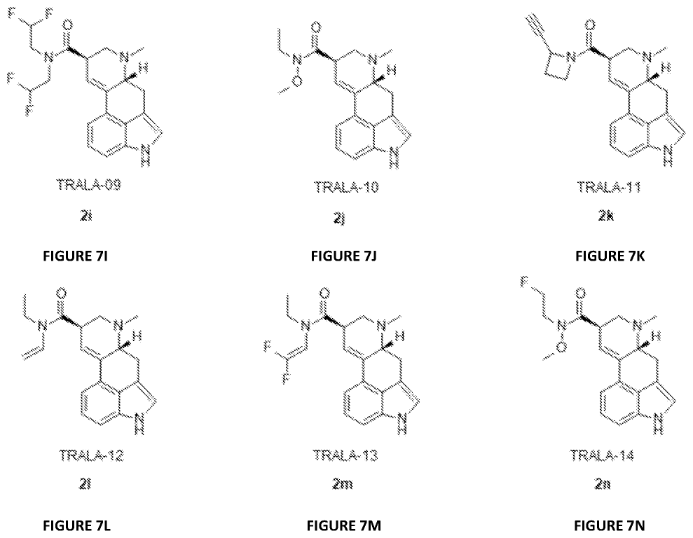

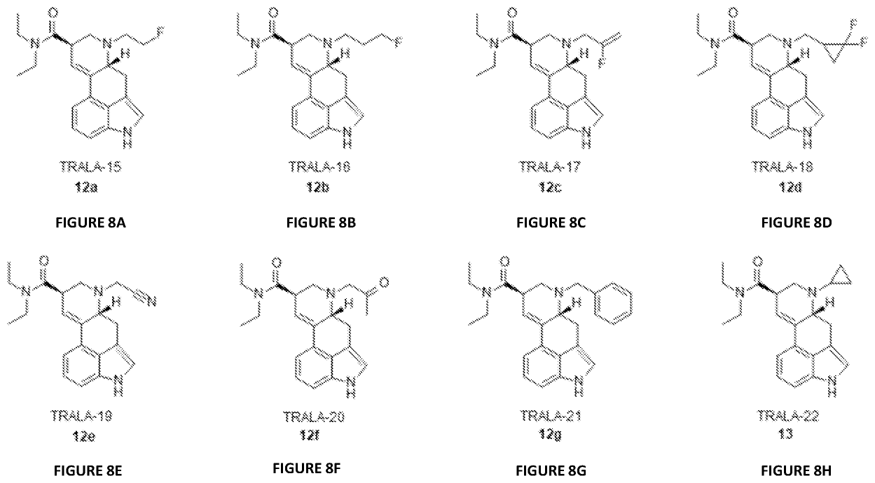

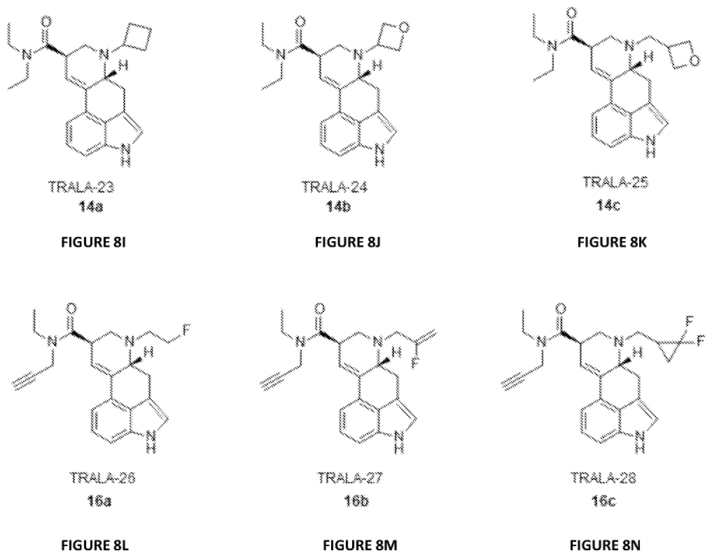

The group presented in the preparation section, namely compounds 2a to 2m, 12a to 12g, 13, 14a to 14c and 16a to 16c, as shown in FIGS. 7A to 8N, is illustrative of lysergic acid derivatives represented in FIG. 1A contemplated within the scope of the invention.

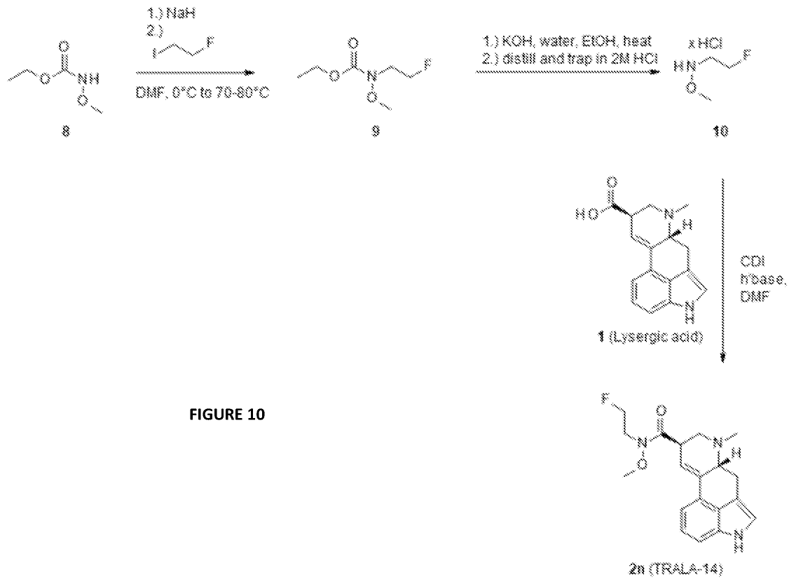

A general access to some the lysergic acid derivatives of the class 1 is outlined in FIGS. 9 to 10. Commercially and synthetically available lysergic acid (1) or lysergic acid monohydrate is activated using an amide coupling reagent such as CDI, TBTU, TCFH, TFFH, T3P, COMU or any other suitable coupling reagent (FIG. 9) in an appropriate solvent such as DMF, dimethylacetamide, DCM or THF, EtOAc, dioxane, acetonitrile or a mixture thereof. Alternatively, activation can also occur with reagents such as POCl₃ or trifluoroacetic anhydride. Next, the activated intermediate is allowed to react with a primary or secondary amine. This amine can be used as free base or as a salt. In case of free base, the amine can be used in excess or as one equivalent to the activated lysergic acid together with a non-nucleophilic base such as triethylamine (NEt₃), N-methylmorpholine (NMM) or N,N-diisopropylethylamine (DIPEA). When the amine to be coupled is applied in a salt form, e.g., as its hydrochloride, it can be used as one equivalent or in excess to the activated lysergic acid, and a non-nucleophilic base such as outlined before can be used to liberate the amine from its salt. The reaction temperature can range from 0-120° C., more favorably 20-100° C. After a reaction time sufficient to allow amide formation the corresponding amide formed is then isolated from the reaction mixture by extraction methods, chromatographic methods or by crystallization of the compound itself or of a salt thereof, or by a combination of these methods.

Compounds from the class 1 (FIG. 2A to FIG. 3H) containing an enamide group (e.g., subclass 1f), can be accessed by different routes (FIG. 9). In one embodiment, a corresponding primary or secondary 2-(phenylthiol)ethylamine is coupled with an activated lysergic acid derivative suitable for amide coupling. The 2-phenylthioethylamine can contain further substituents in any part of the molecule. The corresponding amide containing the 2-(phenylthiol)ethyl group is then oxidized to the corresponding sulfoxide, which is then allowed to react in a thermolysis to yield the corresponding enamide (Taniguchi et al., 2005). The sulfoxide group is chiral and can bear either R or S configuration or be any mixture of stereoisomers. The thermolysis is catalyzed with a suitable base such as NaHCO₃, KHCO₃, Na₂CO₃ or K₂CO₃ and is performed in a suitable solvent such as toluene or di- or trimethylated benzene, such as ortho, meta or para-xylene, but any other solvent chemically inert to the reaction performed can be used, most favorably a xylene. The temperature applied is at 40-200° C., and more favorably at 100-150° C.

The access to the sulfoxide can also be performed as follows. Phenylvinylsulfoxide or a substituted analog is treated with a primary amine R—NH₂ in a suitable organic solvent such as THF, dioxane, ethyl acetate or dichloromethane to form the corresponding N-(2-phenylsulfinylethyl)-R-amine (Hu, Chan, He, Ho, & Wong, 2014). The obtained amine is then coupled with lysergic acid or lysergic acid hydrate as described before to get an amide suitable to undergo thermolysis for enamide formation. As above, the sulfoxide group is chiral and can bear either R or S configuration or be any mixture of stereoisomers.

In another embodiment to access enamides (e.g., subclass 1f), an aldehyde can be coupled with a primary aldehyde to form an imine, which is then coupled (Golding & Wong, 1981; He, Zhao, Wang, & Wang, 2014; Kulyashova & M., 2016; Meuzelaar, van Vliet, Neeleman, Maat, & Sheldon, 1997) with an activated lysergic acid derivative suitable for amide coupling and subsequent elimination. The coupling intermediate is then forced to eliminate to the corresponding enamide.

Another embodiment for accessing such enamides (e.g. subclass 1f), is the formation of oxazolines and subsequent lithiation and alkylation which causes ring opening and formation of an enamide (Xu, Xiao-Yu, Wang, & Tang, 2017).

Further on, enamides (e.g., subclass 1f), can be accessed by direct elimination using, e.g., lithium bis(trimethylsilyl)amide (LiHMDS) (Spiess, Berger, Kaiser, & Maulide, 2021).

Fluorinated enamides (e.g. subclass 1f), as shown in the class 1 (FIG. 2G) can also be accessed by the application of an elimination procedure using a strong base such as butyllithium (BuLi), lithium diisopropylamide (LDA), or LiHDMS onto a 2,2,2-trifluoroethyl or 2-bromo-2,2-difluoroethyl substituent attached to the amide group (Meiresonne, Verniest, De Kimpe, & Mangelinckx, 2015; Riss & Aigbirhio, 2011) of a corresponding lysergamide compound.

The formation of ynamides (e.g., subclass 1g), outlined in the class 1 (FIG. 3A) can be performed, but is not limited to, by an elimination procedure using a strong base such as LiHDMS onto a 2,2,2-trifluoroethyl or 2-bromo-2,2-difluoroethyl substituent attached to the amide group (Meiresonne et al., 2015) of a corresponding lysergamide compound.

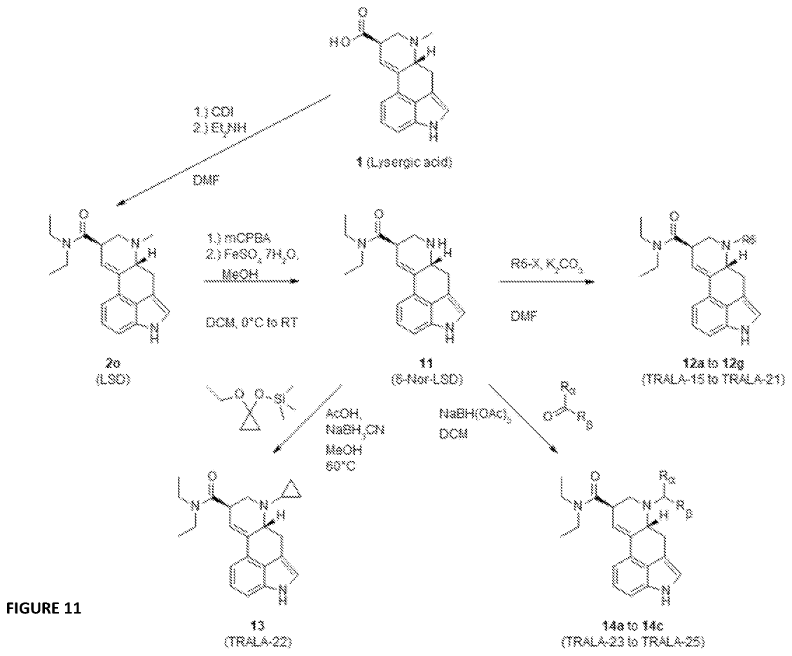

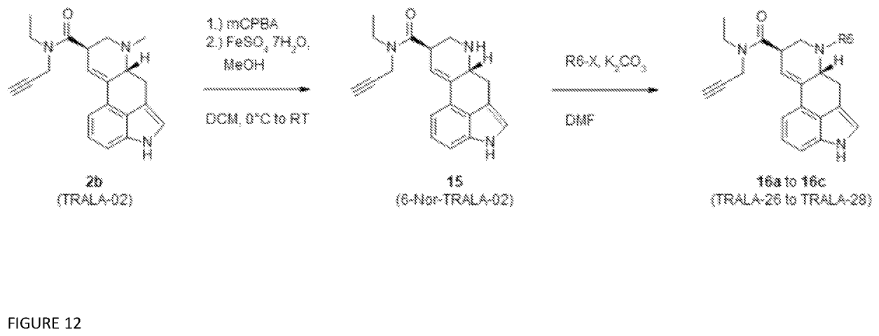

Compounds of the class 2 (FIG. 4A to FIG. 5C) can be accessed via the corresponding 6-Nor-LSD or any other 6-Nor compound as starting material. The preparation of 6-Nor-LSD (FIG. 11) is well documented for applying the classical Von-Braun reaction, wherein cyanogen bromide in boiling tetrachloromethane is applied, both highly problematic compounds to handle, and the formed aminonitrile is then reduced by elemental zinc in acetic acid (Fehr et al., 1970). Herein, another method was used, described in WO2006128658A1, where a pyrrolidine analog of LSD was prepared. In analogy to this, LSD is treated with an oxidizing agent such as meta-chloroperoxybenzoic acid (mCPBA) in dichloromethane under cooling and then the formed N-oxide is reduced by adding FeSO₄. This is considerably safer, easy to handle and a very quick reaction. Furthermore, yields are comparable or even superior to the Von-Braun reaction. Other oxidants such as H₂O₂ or cumolhydroperoxide in an organic solvent such as an alcohol, ethyl acetate or dichloromethane, can also be used. After an isolation step, which can be performed by extraction methods, chromatographic methods or by crystallization methods of the compound itself or of a salt thereof, or by a combination of these methods, the obtained 6-Nor-LSD, or any other 6-Nor compound such as 6-Nor-TRALA-02, e.g., as shown in FIG. 12, is used in reactions to access compounds represented by the class 2 (FIG. 4A to FIG. 5C). The recovered LSD or any other recovered lysergic acid derivative from the above N-demethylation reaction can be reused in the N-demethylation reaction as outlined above to further increase the amount of desired 6-Nor-LSD or other 6-Nor compound. This cyclic process can be repeated as many times as technique and/or required amounts of the compounds allows.

Some compounds represented by class 2 as represented in FIG. 4A to FIG. 5C can be accessed by allowing to react 6-Nor-LSD or another 6-Nor compound with a correspondingly substituted R6 containing a leaving group to be substituted with the basic N6 nitrogen of 6-Nor-LSD or of another 6-Nor compound. The leaving group can be, e.g., a halogen, a mesylate, tosylate, or a triflate. Further on, a reductive amination can also be applied by using a suitable carbonyl compound and a reducing compound such as NaBH₄ or Na(OAc)₃BH or NaBH₃CN, but also hydrogen in presence or absence of a catalyst, in a suitable solvent.

For some R6 substituents to be introduced bearing strong electron-withdrawing substituents the secondary N6 of 6-Nor-LSD or other lysergic acid derivatives with N—H in 6-position the nucleophilic character of the secondary amine may not be sufficient high for use as a nucleophile. In such cases, the lysergic acid derivative has either to be protected adequately to selectively deprotonate N6 or the electrophile must be activated. In such a way it can be helpful to use transition metals or transition metal oxides or salts such as silver salts to accelerate N-alkylation. Favorably AgNO₃ or AgOTf (AgCF₃SO₃) is added to the reaction mixture of the corresponding secondary N6 amine and alkylating agent in an organic solvent such as tetrahydrofuran (THF), dioxane, an alcohol such as methanol (MeOH), ethanol (EtOH), isopropyl alcohol (iPrOH) or dichloromethane (DCM). The mixture can be held at 0-100° C., more favorably at 20-100° C.

It is well known that due to the extremely deactivated reactivity (i.e., due to the electron-withdrawing properties of fluorine), in certain cases a 2,2,2-trifluoroethyl substituent cannot be simply introduced into an amine by applying one of the above conditions, and even 2,2,2-trifluoroethyl triflate, a compound of much more reactivity than 2,2,2-trifluoroethyl iodide, shows extremely low reactivity in nucleophilic substitutions with certain amines. Such substituents can be introduced onto an amine by using a synthetic equivalent, namely and exemplarily, 2,2,2-trifluoroacetaldehyde ethyl hemiacetal (alternative name: 1-ethoxy-2,2,2-trifluoro-ethanol) (Mimura, Kawada, Yamshita, Sakamato, & Kikugawa, 2010). The intermediate formed is then reduced with a suitable reducing agent such as NaBH₄ or Na(OAc)₃BH or NaBH₃CN.

Enamine compounds represented by class 2 (subclass 2f) as represented in FIG. 4G can be accessed by allowing to react a corresponding carbonyl compound with 6-Nor-LSD to form an imine by removing or absorbing water, and, where necessary, an additional non-nucleophilic base is applied. Alternatively, such enamines can also be accessed by allowing to react a fluorinated 1-halo-1-alkene with 6-Nor-LSD or another 6-Nor compound in a direct halo-substitution reaction (WO2006046417A1).

Furthermore, enamine compounds represented by class 2 (subclass 2f) as represented in FIG. 4G can be accessed by e.g., allowing to react (Riss & Aigbirhio, 2011) an N6-substituted N6-2,2,2-trifluoroethyl-6-Nor-LSD with a strong base such as LDA or BuLi.

Alkoxyamine compounds, also known as N-hydroxyethers, represented by class 2 (subclass 2h) as represented in FIG. 5B can be accessed by allowing to react 6-Nor-LSD with an oxidizing reagent such as H₂O₂ or mCPBA in an organic solvent to form the corresponding 6-Nor-LSD-N6-hydroxylamine, then deprotonating the N6-hydroxylamine with a base such as LDA, KOtBU, BuLi or LiHDMS, and then allowing this deprotonated intermediate to react with a correspondingly substituted R6 containing a leaving group to be substituted with the deprotonated oxygen of 6-Nor-LSD-N6-hydroxylamine. The leaving group can be a halogen, a mesylate, tosylate or a triflate or other suitable leaving groups.

Arylamines or heteroarylamines represented by class 2 (subclass 2i) as represented in FIG. 5C can be accessed by allowing to react 6-Nor-LSD or another 6-Nor compound with corresponding aryl or heteroaryl halides, triflates, as, e.g., described generally as the Buchwald-Hartwig-amination.

Benzylamines and (heteroarylmethyl)amines represented by class 2 (subclass 2i) as represented in FIG. 5C can be accessed by allowing to react 6-Nor-LSD or another 6-Nor compound with corresponding aryl or (heteroarylmethyl) halides in a classical substitution reaction or with the corresponding aldehydes in a reductive amination way, by applying reductive conditions such as NaBH₄ or Na(OAc)₃BH or NaBH₃CN, but also hydrogen, in the presence or absence of a catalyst, in a suitable solvent.

N6 substituents can also consist of a cycloalkane or oxacycloalkane (subclass 2d and 2g in FIG. 4E and FIG. 5A). These substituents can be introduced by reductive aminations with 6-Nor-LSD or another 6-Nor compound and a corresponding oxo-cycloalkane or oxo-oxacycloalkane and a reducing compound such as NaBH₄ or Na(OAc)₃BH or NaBH₃CN, but also hydrogen in presence or absence of a catalyst, in a suitable solvent.

Further on, these cycloalkane substituents represented by the subclass 2d and 2g in FIG. 4E and FIG. 5A can also be introduced by a substitution reaction by allowing to react 6-Nor-LSD or another 6-Nor compound with a correspondingly substituted R6 cycloalkane or oxacycloalkane containing a leaving group to be substituted with the basic N6 nitrogen of 6-Nor-LSD. The leaving group can be, e.g., a halogen, a mesylate, tosylate or a triflate.

Yet another access to compounds represented by the subclass 2d and 2g in FIG. 4E and FIG. 5A is achieved by the use of 6-Nor-LSD or another 6-Nor compound, allowed to be reacted with a cycloalkane or oxacycloalkane containing a geminal substituted alkoxy-(trialkylsilyloxy) substitution, under acidic conditions such as the use of acetic acid and by using a reducing compound such as NaBH₄ or Na(OAc)₃BH or NaBH₃CN, but also hydrogen in presence or absence of a catalyst, in a suitable solvent, can be applied.

Compounds of the class 3 (FIG. 6A) can be accessed by the combination of any of the aforementioned synthetic routes. In addition to that, one can also first introduce a suitable protecting group in either N1, N6 or on the carboxylic function attached to C8 of the ergoline structure on LSD, 6-Nor-LSD, or any other Nor-derivative such as N6-Nor-ergotamine, N6-deprotected intermediate, N6-deprotected lysergic acid, N6-deprotected lysergic acid ester or suitably converted compound.

Compounds of the class 4 (FIG. 6B) can be accessed by corresponding functionalization of N1 of the ergoline core structure by using a compound of class 1, class 2, or class 3 and vice versa.

Compounds of the class 5 (FIG. 6C) can be accessed by corresponding functionalization of N1 of the ergoline core structure by using a compound of class 1, class 2, or class 3, bearing at least one deuteron atom at the ergoline core structure and vice versa.

A selection of the synthesized lysergic acid derivatives is being investigated at the key target for psychoactive effects in vitro (Liechti et al. data on file). The main target of psychedelics is the 5-HT2A receptor (Holze, Vizeli, et al., 2021) and typically there is a high affinity binding at this receptor (Rickli et al., 2016). Additionally, the binding potency at the 5-HT2A receptor is typically predictive of the human doses of psychedelics to be psychoactive for many compounds (Luethi & Liechti, 2018). Furthermore, the psychedelic effects of psilocybin in humans have been shown to correlate with 5-HT2A receptor occupancy measures using positron emission tomography (Madsen et al., 2019). Thus, interactions with this target are relevant and predict psychedelic action with high likelihood for most psychedelics. However, this may not be the case for all substances within this class.

Additional receptors such as the serotonergic 5-HT1A and 5-HT2C or dopaminergic D2 receptors are thought to moderate the effects of psychedelics (Rickli et al., 2016). Although some psychedelics like psilocybin do not directly act on dopaminergic receptors, they have nevertheless some dopaminergic properties by releasing dopamine in the striatum (Vollenweider, Vontobel, Hell, & Leenders, 1999) likely via 5-HT1A receptor activation (Ichikawa & Meltzer, 2000). Furthermore, LSD has activity at D2 receptors (Rickli et al., 2016) and some of its behavioral effects in animals may be linked to this target (Marona-Lewicka, Thisted, & Nichols, 2005) although the acute psychoactive effects in humans are mainly if not fully mediated via 5-HT2 receptor (Holze, Vizeli, et al., 2021; Preller et al., 2017).

Activity of compounds at monoamine transporters are thought to mediate MDMA-like empathogenic effects (Hysek et al., 2012). Importantly, LSD is a high affinity 5-HT2A receptor ligand and extremely low doses are needed to induce psychoactive effects in humans. Even doses at 0.1 mg or below can have extraordinarily strong psychedelic effects in humans and the same is likely the case for the substances developed within the present invention although higher or lower potency is also possible in some lysergic acid compounds, to be evaluated in detail clinically. Key results of the preliminary pharmacological profiling of the compounds described herein were:

Some of the lysergic acid derivatives represented in FIG. 1A showed high binding affinity in ongoing studies at the serotonin 5-HT2A receptor indicating activity as psychedelics.

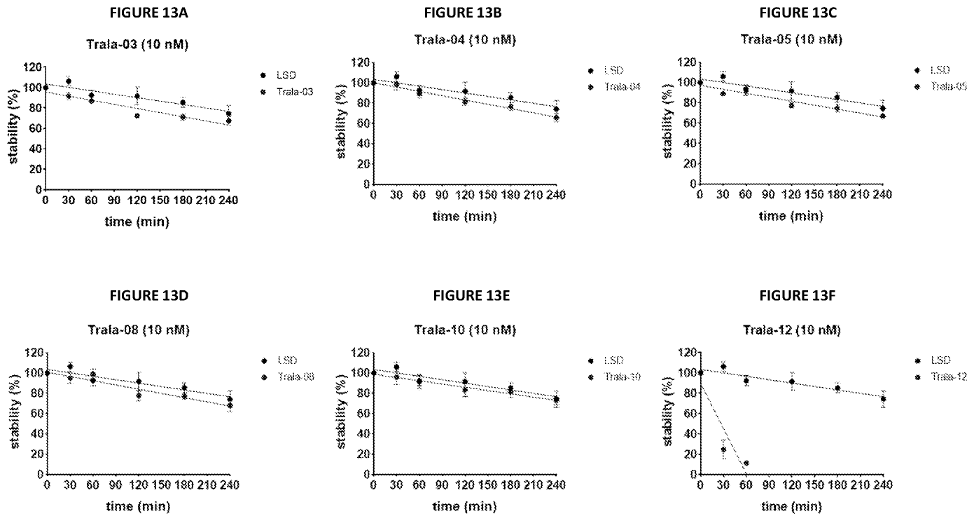

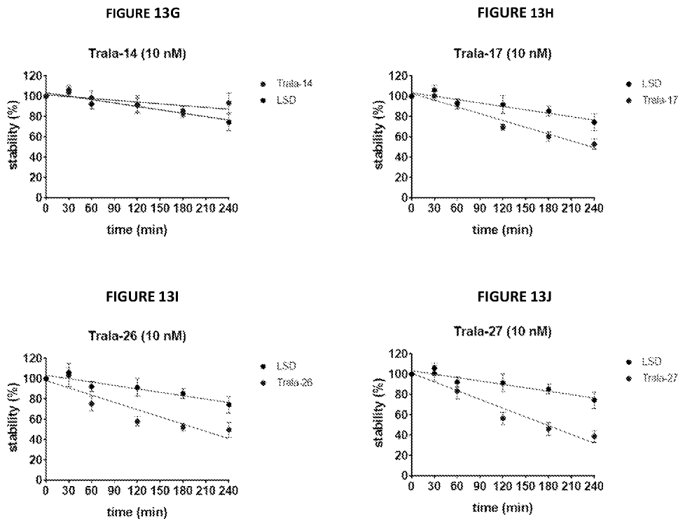

A microsomal investigation of some of the lysergic acid derivatives represented in FIG. 1A was conducted and revealing different metabolic stability in comparison to LSD as shown in FIGS. 13A-13J. In particular, the derivatives 21 (TRALA-12), 12c (TRALA-17), 16a (TRALA-26), and 16b (TRALA-27) were significantly faster metabolized than LSD in microsomal incubations over 4 hours. Substance 21 displayed by far the fastest metabolism with the majority of substance being metabolized after only 30 minutes. Given its fast metabolism, it is likely not active orally but is a candidate for intravenous administration in particular as an infusion and expected to then produce short-lasting or also maintained effects that can rapidly be terminated upon stopping the infusion. The metabolic profile of 12c, 16a, and 16b, which all displayed a microsomal metabolism significantly faster than LSD, appear to be promising among the tested derivatives for oral administration with an expected shorter duration of action compared with LSD. However, it is important to note that in addition to the metabolic profile, other factors such as 5-HT2A receptor activity are important to consider when choosing promising drug candidates for therapeutic applications. In addition, in vivo experiments are necessary further assessing the clinical pharmacokinetics of these substances.

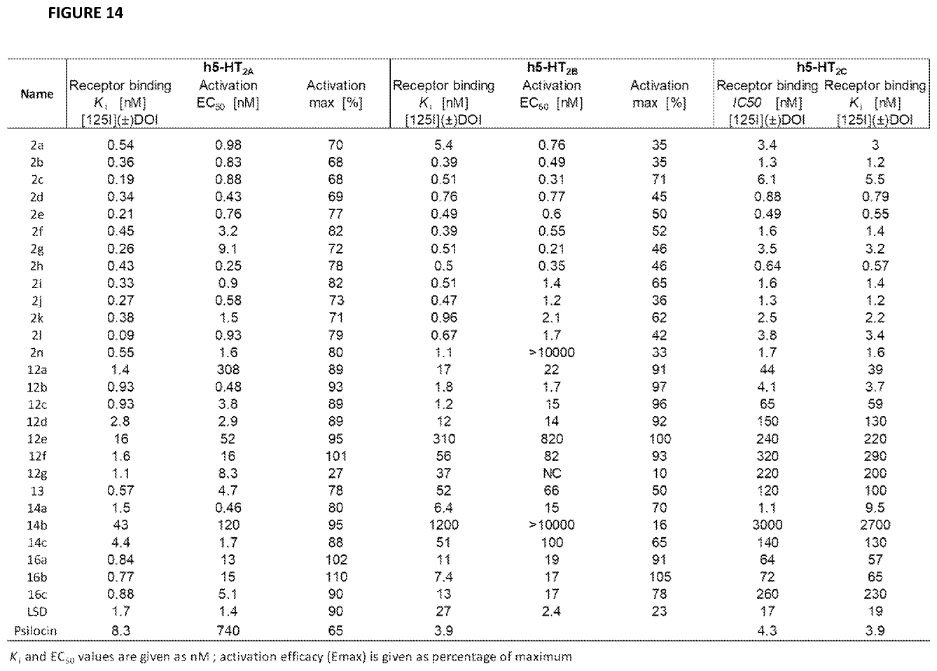

Receptor interaction profiles of the novel substances at the key targets and compared with LSD and psilocin (the active metabolite of the prodrug and psychedelic psilocybin) were determined and are shown in FIG. 14. Many of the novel compounds exhibited higher binding affinity compared with LSD at the receptor responsible for the psychedelic action of psychedelics (h5-HT2A) as evidenced by similar or lower Ki values as for LSD. Similarly, many of the novel compounds also exhibited similar or greater receptor activation potency compared with LSD at the 5-HT2A receptor responsible for their acute and therapeutic actions and as evidenced by lower or similar EC50 values for h5-HT2A receptor activation compared with LSD. Several of the novel compounds showed lower binding potency and/or receptor activation potency at the 5-HT2B receptor compared with LSD indicating a similar or reduced risk of cardiac toxicity (cardiac valve fibrosis) since the 5-HT2B receptor is thought to mediate this adverse effect of serotonergic agents when high doses are used chronically.

Together, the in vitro profiles of lysergic acid derivatives represented in FIG. 1A compared with that of psilocin and LSD indicate overall psychedelic properties when used in humans. Accordingly, some lysergic acid derivatives can exert psychedelic acute effect profiles that are more beneficial to some patients including but not limited to: more overall positive effects, varying perceptual effects, more emotional effects, less anxiety, less cardio-stimulant effects, less adverse effects, less nausea, longer as well as shorter effects among other properties and compared to LSD.

There are several problems when using LSD that can be solved using the compounds described herein. Namely, a long duration of action of psychedelic experience can be limited in some cases. Derivatives represented in FIG. 1A can be more prone to metabolism and thus cause shorter duration of action. Further on, psychedelics like psilocybin or LSD can produce adverse effects including nausea and vomiting, cardiovascular stimulation, and an increase in body temperature and others. The novel compounds can produce less nausea, less cardio stimulation, less thermogenesis and/or other adverse responses. LSD has a long duration of action. The presently developed substances were designed to have similar qualitative effects to LSD while acting shorter or to have a long duration of action but other qualitative effects as reflected by their structural changes and associated pharmacological properties. In particular, metabolically less-stable compounds were created to shorten the plasma half-life and duration of action in humans. Other alterations of the chemical structure were designed to create substances with qualitative effects different from those of LSD and creating subjective effects that are considered beneficial to assist psychotherapy including feelings of empathy, openness, trust, insight, and connectedness and known to those knowledgeable in the field.

The compounds represented by FIG. 1A act with shorter, with similar or with longer duration of action in human in comparison to the original LSD molecule. This is triggered by modification of the molecular structure in FIG. 1A.

The group presented in the preparation section, namely compounds 2a to 2m, 12a to 12g, 13, 14a to 14c and 16a to 16c (see FIGS. 7A-7N and FIGS. 8A-8N), is illustrative of lysergic acid derivatives represented in FIG. 1A contemplated within the scope of the invention.

The compounds according to the invention and represented in FIG. 1A allow modification of the mode of action, the psychodynamic processes, and the qualitative perceptions, e.g., in terms of psychedelic or empathogenic intensity in comparison to the original LSD molecule.

The compounds according to the invention and represented in FIG. 1A can cause similar or different quality of imagery, fantasy and closed or open eyes visuals in comparison to the original LSD molecule.

The compounds according to the invention and represented in FIG. 1A can have a similar, lower or a higher dose potency in comparison to the original LSD molecule.

The compounds according to the invention and represented in FIG. 1A can cause similar or more favorable body feelings in comparison to the original LSD molecule.

The modified properties can be tailored and applied individually to the patient's need. This is not only targeted by changing the compound's receptor profile but also greatly by the modification of ADME (Absorption, Distribution, Metabolism and Excretion) via the introduction of more, similar, or less liable substituents in positions N1, N6 or in the carboxamide attached to C8 of the ergoline structure, as in compounds represented in FIG. 1A. In addition, stabilities can also be modified by the introduction of one or more deuteron in the ergoline core structure, as represented by class 5 in FIG. 6C.

Preparation of the Compounds

A general access to some the lysergic acid derivatives of the class 1 is outlined in FIGS. 9 to 10. Commercially and synthetically available lysergic acid (1) or lysergic acid monohydrate is activated using an amide coupling reagent such as CDI, TBTU, TCFH, TFFH, T3P, COMU or any other suitable coupling reagent (FIG. 9) in an appropriate solvent such as DMF, dimethylacetamide, DCM or THF, EtOAc, dioxane, acetonitrile or a mixture thereof. Alternatively, activation can also occur with reagents such as POCl₃ or trifluoroacetic anhydride. Next, the activated intermediate is allowed to react with a primary or secondary amine. This amine can be used as free base or as a salt. In case of free base, the amine can be used in excess or as one equivalent to the activated lysergic acid together with a non-nucleophilic base such as triethylamine (NEt₃), N-methylmorpholine (NMM) or N,N-diisopropylethylamine (DIPEA). When the amine to be coupled is applied in a salt form, e.g., as its hydrochloride, it can be used as one equivalent or in excess to the activated lysergic acid, and a non-nucleophilic base such as outlined before can be used to liberate the amine from its salt. The reaction temperature may range from 0-120° C., more favorably 20-100° C. After a reaction time sufficient to allow amide formation the corresponding amide formed is then isolated from the reaction mixture by extraction methods, chromatographic methods or by crystallization of the compound itself or of a salt thereof, or by a combination of these methods.

Compounds from the class 1 (FIG. 2A to FIG. 3H) containing an enamide group (e.g., subclass 1f), can be accessed by different routes (FIG. 9). In one embodiment, a corresponding primary or secondary 2-(phenylthiol)ethylamine is coupled with an activated lysergic acid derivative suitable for amide coupling. The 2-phenylthioethylamine can contain further substituents in any part of the molecule. The corresponding amide containing the 2-(phenylthiol)ethyl group is then oxidized to the corresponding sulfoxide, which is then allowed to react in a thermolysis to yield the corresponding enamide (Taniguchi et al., 2005). The sulfoxide group is chiral and can bear either R or S configuration or be any mixture of stereoisomers. The thermolysis is catalyzed with a suitable base such as NaHCO₃, KHCO₃, Na₂CO₃ or K₂CO₃ and is performed in a suitable solvent such as toluene or di- or trimethylated benzene, such as ortho, meta or para-xylene, but any other solvent chemically inert to the reaction performed can be used, most favorably a xylene. The temperature applied is at 40-200° C., and more favorably at 100-150° C.

The access to the sulfoxide can also be performed as follows. Phenylvinylsulfoxide or a substituted analog is treated with a primary amine R—NH₂ in a suitable organic solvent such as THF, dioxane, ethyl acetate or dichloromethane to form the corresponding N-(2-phenylsulfinylethyl)-R-amine (Hu et al., 2014). The obtained amine is then coupled with lysergic acid or lysergic acid hydrate as described before to get an amide suitable to undergo thermolysis for enamide formation. As above, the sulfoxide group is chiral and can bear either R or S configuration or be any mixture of stereoisomers.

In another embodiment to access enamides (e.g., subclass 1f), an aldehyde can be coupled with a primary aldehyde to form an imine, which is then coupled (Golding & Wong, 1981; He et al., 2014; Kulyashova & M., 2016; Meuzelaar et al., 1997) with an activated lysergic acid derivative suitable for amide coupling and subsequent elimination. The coupling intermediate is then forced to eliminate to the corresponding enamide.

Another embodiment for accessing such enamides (e.g., subclass 1f), is the formation of oxazolines and subsequent lithiation and alkylation which causes ring opening and formation of an enamide (Xu et al., 2017).

Further on, enamides (e.g. subclass 1f), can be accessed by direct elimination using, e.g., LiHMDS (Spiess et al., 2021).

Fluorinated enamides (e.g., subclass 1f), as shown in the class 1 (FIG. 2A to FIG. 3H) can also be accessed by the application of an elimination procedure using a strong base such as BuLi, LDA or LiHDMS onto a 2,2,2-trifluoroethyl or 2-bromo-2,2-difluoroethyl substituent attached to the amide group (Meiresonne et al., 2015; Riss & Aigbirhio, 2011) of a corresponding lysergamide compound.

The formation of ynamides (e.g., subclass 1g), outlined in the class 1 (FIG. 2A to FIG. 3H) can be performed, but is not limited to, by an elimination procedure using a strong base such as LiHDMS onto a 2,2,2-trifluoroethyl or 2-bromo-2,2-difluoroethyl substituent attached to the amide group (Meiresonne et al., 2015) of a corresponding lysergamide compound.

Compounds of the class 2 (FIG. 4A to FIG. 5C) can be accessed via the corresponding 6-Nor-LSD or any other 6-Nor compound as starting material. The preparation of 6-Nor-LSD (FIG. 11) is well documented for applying the classical Von-Braun reaction, wherein cyanogen bromide in boiling tetrachloromethane is applied, both highly problematic compounds to handle, and the formed aminonitrile is then reduced by elemental zinc in acetic acid (Fehr et al., 1970). Herein, another method was used, described in WO2006128658A1, where a pyrrolidine analog of LSD was prepared. In analogy to this, LSD is treated with an oxidizing agent such as mCPBA in dichloromethane under cooling and then the formed N-oxide is reduced by adding FeSO₄. This is considerably safer, easy to handle and a very quick reaction. Furthermore, yields are comparable or even superior to the Von-Braun reaction. Other oxidants such as H₂O₂ or cumolhydroperoxide in an organic solvent such as an alcohol, ethyl acetate or dichloromethane, can also be used. After an isolation step, which can be performed by extraction methods, chromatographic methods or by crystallization methods of the compound itself or of a salt thereof, or by a combination of these methods, the obtained 6-Nor-LSD or any other 6-Nor compound such as 6-Nor-TRALA-02 (e.g., as shown in FIG. 12) is used in reactions to access compounds represented by the class 2 (FIG. 4A to FIG. 5C). The recovered LSD or any other recovered lysergic acid derivative from the above N-demethylation reaction can be reused in the N-demethylation reaction as outlined above to further increase the amount of desired 6-Nor-LSD or other 6-Nor compound. This cyclic process can be repeated as many times as technique and/or required amounts of the compounds allows.

Some compounds represented by class 2 as represented in FIG. 4A to FIG. 5C can be accessed by allowing to react 6-Nor-LSD or another 6-Nor compound with a correspondingly substituted R6 containing a leaving group to be substituted with the basic N6 nitrogen of 6-Nor-LSD or of another 6-Nor compound. The leaving group can, e.g., be a halogen, a mesylate, tosylate or a triflate. Further on, a reductive amination can also be applied by using a suitable carbonyl compound and a reducing compound such as NaBH₄ or Na(OAc)₃BH, or NaBH₃CN, but also hydrogen in presence or absence of a catalyst, in a suitable solvent.

For some R6 substituents to be introduced bearing strong electron-withdrawing substituents the secondary N6 of 6-Nor-LSD or other lysergic acid derivatives with N—H in 6-position the nucleophilic character of the secondary amine may not be sufficient high for use as a nucleophile. In such cases, the lysergic acid derivative has either to be protected adequately to selectively deprotonate N6 or the electrophile has to be activated. In such a way it can be helpful to use transition metals or transition metal oxides or salts such as silver salts to accelerate N-alkylation. Favorably AgNO₃ or AgOTf (AgCF₃SO₃) is added to the reaction mixture of the corresponding secondary N6 amine and alkylating agent in an organic solvent such as THF, dioxane, an alcohol such as MeOH, EtOH, iPrOH or DCM. The mixture can be held at 0-100° C., more favorably at 20-100° C.

It is well known that due to the extremely deactivated reactivity, i.e., due to the electron-withdrawing properties of fluorine, in certain cases a 2,2,2-trifluoroethyl substituent cannot be simply introduced into an amine by applying one of the above conditions, and even 2,2,2-trifluoroethyl triflate, a compound of much more reactivity than 2,2,2-trifluoroethyl iodide, shows extremely low reactivity in nucleophilic substitutions with amines. Such substituents can be introduced onto an amine by using a synthetic equivalent, namely and exemplarily, 2,2,2-trifluoroacetaldehyde ethyl hemiacetal (alternative name: 1-ethoxy-2,2,2-trifluoro-ethanol) (Mimura et al., 2010). The intermediate formed is then reduced with a suitable reducing agent such as NaBH₄, Na(OAc)₃BH, or NaBH₃CN.

Enamine compounds represented by class 2 (subclass 2f) as represented in FIG. 4G can be accessed by allowing to react a corresponding carbonyl compound with 6-Nor-LSD to form an imine by removing or absorbing water, and, where necessary, an additional non-nucleophilic base is applied. Alternatively, such enamines can also be accessed by allowing to react a fluorinated 1-halo-1-alkene with 6-Nor-LSD or another 6-Nor compound in a direct halo-substitution reaction (WO2006046417A1).

Furthermore, enamine compounds represented by class 2 (subclass 2f) as represented in FIG. 4G can be accessed by e.g., allowing to react (Riss & Aigbirhio, 2011) an N6-substituted N6-2,2,2-trifluoroethyl-6-Nor-LSD with a strong base such as LDA or BuLi.

Alkoxyamine compounds, also known as N-hydroxyethers, represented by class 2 (subclass 2h) as represented in FIG. 5B can be accessed by allowing to react 6-Nor-LSD with an oxidizing reagent such as H₂O₂ or mCPBA in an organic solvent to form the corresponding 6-Nor-LSD-N6-hydroxylamine, then deprotonating the N6-hydroxylamine with a base such as LDA, KOtBU, BuLi or LiHDMS, and then allowing this deprotonated intermediate to react with a correspondingly substituted R6 containing a leaving group to be substituted with the deprotonated oxygen of 6-Nor-LSD-N6-hydroxylamine. The leaving group can be a halogen, a mesylate, tosylate or a triflate or other suitable leaving groups.

Arylamines or heteroarylamines represented by class 2 (subclass 2i) as represented in FIG. 5C can be accessed by allowing to react 6-Nor-LSD or another 6-Nor compound with corresponding aryl or heteroaryl halides, triflates, as, e.g., described generally as the Buchwald-Hartwig-amination.

Benzylamines and (heteroarylmethyl)amines represented by class 2 (subclass 2i) as represented in FIG. 5C can be accessed by allowing to react 6-Nor-LSD or another 6-Nor compound with corresponding aryl or (heteroarylmethyl) halides in a classical substitution reaction or with the corresponding aldehydes in a reductive amination way, by applying reductive conditions such as NaBH₄, Na(OAc)₃BH, or NaBH₃CN, but also hydrogen, in the presence or absence of a catalyst, in a suitable solvent.

N6 substituents can also consist of a cycloalkane or oxacycloalkane (subclass 2d and 2g in FIG. 4E and FIG. 5A). These substituents can be introduced by reductive aminations with 6-Nor-LSD or another 6-Nor compound and a corresponding oxo-cycloalkane or oxo-oxacycloalkane and a reducing compound such as NaBH₄, Na(OAc)₃BH, or NaBH₃CN, but also hydrogen in presence or absence of a catalyst, in a suitable solvent.

Further on, these cycloalkane substituents represented by the subclass 2d and 2g in FIG. 4E and FIG. 5A can also be introduced by a substitution reaction by allowing to react 6-Nor-LSD or another 6-Nor compound with a correspondingly substituted R6 cycloalkane or oxacycloalkane containing a leaving group to be substituted with the basic N6 nitrogen of 6-Nor-LSD. The leaving group can be, e.g., a halogen, a mesylate, tosylate or a triflate.

Yet another access to compounds represented by the subclass 2d and 2g in FIG. 4E and FIG. 5A is achieved by the use of 6-Nor-LSD or another 6-Nor compound, allowed to be reacted with a cycloalkane or oxacycloalkane containing a geminal substituted alkoxy-(trialkylsilyloxy) substitution, under acidic conditions such as the use of acetic acid and by using a reducing compound such as NaBH₄, Na(OAc)₃BH, or NaBH₃CN, but also hydrogen in presence or absence of a catalyst, in a suitable solvent, can be applied.

Compounds of the class 3 (FIG. 6A) can be accessed by the combination of any of the aforementioned synthetic routes. In addition to that, one can also first introduce a suitable protecting group in either N1, N6 or on the carboxylic function attached to C8 of the ergoline structure on LSD, 6-Nor-LSD or any other Nor-derivative such as N6-Nor-ergotamine, N6-deprotected intermediate, N6-deprotected lysergic acid, N6-deprotected lysergic acid ester or suitably converted compound.

Compounds of the class 4 (FIG. 6B) can be accessed by corresponding functionalization of N1 of the ergoline core structure by using a compound of class 1, class 2, or class 3, or vice versa.

Compounds of the class 5 (FIG. 6C) can be accessed by corresponding functionalization of N1 of the ergoline core structure by using a compound of class 1, class 2, or class 3, bearing at least one deuteron atom at the ergoline core structure, or vice versa.

Detailed Description of the Chemical Preparation of the Compounds

General preparation and equipment information: NMR was performed on a Bruker NMR (¹H: 300 MHz and ¹⁹F: 282 MHz) at ambient temperature. Reaction controls were performed by silica gel TLC (F254; UV detection) and HPLC UV & MS (Agilent 1100, UV at 210 nm and 313 nm, Waters SQD, ESI+ mode) under basic as well as acidic conditions (solvents: A: either 0.02% NH₄OH in water or 0.05% TFA in water, B: acetonitrile, gradients from 5% to 95% B, reversed phase C18 HPLC column). All reactions, workups, drying steps, and storing were performed under exclusion of daylight and of electric light such as neon lighting or light bulbs containing wavelengths of the blue and/or UV spectrum. Working was performed in either light-excluding lab ware or under orange to red light (UV free LED). This helps to prevent decompositions of the compounds. Further on, reactions und some purifications can be performed under protecting gas such as Nitrogen or Argon to further protect the compounds from decompositions. Purifications were performed by using silica gel column chromatography and organic solvents such as mixtures of an alcohol (MeOH or EtOH) and dichloromethane, and in some cases, 0.1% to 1% of NH₄OH 25% or 0.1% to 1% NEt₃ was added. In a similar way, preparative TLC (silica gel) using the aforementioned solvents was applied as well. Working safety: a suitable personal protecting ware common to lab works and fume hoods with a movable glass window were used. Possible contaminations on e.g., gloves or surfaces can quickly be detected by having a long-wave UV lamp (e.g., 366 nm) at hands, preventing further distribution of active materials. Typically, compounds bearing an intact lysergic acid amide substructure, show a strong blueish fluorescence even in trace amounts. Quick deactivation of the potential central effects of these compounds can be performed by using, e.g., a mixture of bleach and diluted alcohol.

An appropriate purity as well as identity check and determination of epimeric identity is crucial to evaluate the compounds of invention for their biological properties. The inventors did not rely on TLC or single HPLC analysis but instead set up a deeper evaluation of analyses, since for some compounds TLC or single HPLC is not sufficient to judge.

Purity check of the final compounds was performed on two different HPLC systems with different columns and different eluents, at 195 nm as well as at 313 nm (reaction controls: 210 nm and 313 nm). The very low absorption wavelength reveals any organic contaminants, and the higher wavelength corresponds approximatively to a characteristic local maximum of these compounds and would reveal whether there would by any structurally related contaminations. Further on, ¹H-NMR and, where applicable, by ¹⁹F-NMR helped further to judge purities.

Identity check of the final compounds was performed by HPLC-MS as well as by ¹H-NMR and, where applicable, by ¹⁹F-NMR. For important notes to NMR analysis and interpretations, see the following instructions.

General method for the amide couplings. To a suspension of 286 mg (1 mmol) lysergic acid monohydrate (note: the water-free lysergic acid can be used similarly) in 4 mL DMF anhydr. were added 258 mg (1.59 mmol) 1,1′-carbonyldiimidazole (CDI) in one portion. The suspension became clear after a few minutes, and an HPLC-UV and -MS based activation check after 30 min by dissolving a minimal sample of reaction mixture in MeOH anhydr. (important) indicated clean and complete methyl ester formation (lysergic acid methyl ester appeared as two epimers). Thus, the amine to be coupled (1.05 to 1.5 eq) as either free base or as hydrochloride salt was premixed with diisopropylethylamine (DIPEA; 435 μL, 2.5 eq) in 2 mL DMF anhydr. and the clear amine solution was added all at once to the above activated lysergic acid solution. Note: in some cases, a large excess of the free amine (up to 10 eq, no DIPEA) did force the reaction to give C8-epimeric ratios much more towards the desired and pharmacologically active 8R-carboxamide epimer. This way was used of forcing the epimeric ratio depending on availability of amines to be used and on economic reasons. After the reaction control (by TLC: DCM/MeOH 9/1 and HPLC MS and UV at 210 nm and 313 nm) indicated complete or near complete conversion (note: in any case there was formed a mixture of C8-epimers in ratios ranging from approx. 8R:8S=8:2 to 4:6, based on interpretation of UV absorption at 313 nm. For amines with low nucleophilicity such as di(2,2-difluoroethyl)amine up to four days reaction time was needed), the DMF was either removed in vacuo at 40° C. using a strong vacuum pump before extraction as following was performed, or the reaction mixture was directly partitioned between water (40 mL) and 40 mL heptane/EtOAc 1:1 (40 mL). The layers were separated, and the very dark aq. layer was further extracted with the heptane/EtOAc 1:1 (2×20 mL). The combined, cognac-colored org. layers were further washed with water (3×20 mL) and dried by slowly filtering them through a Na₂SO₄ pad. After evaporation of the org. volatiles there was obtained a green to brown residue as crude product. This was purified by either column chromatography or prep. TLC as described under the chapter General to get the corresponding lysergic acid amide derivative as free base. As a general observation and in agreement with (Bailey, Verner, & Legault, 1973; Hoehn, Nichols, McCorvy, Neven, & Kais, 2017; Hoffman & Nichols, 1985; Stachulski, Nichols, & Scheinmann, 1996), on normal phase chromatographic conditions, the first compound eluted from silica gel showing blue fluorescence under long-wave UV corresponded to the pharmacologically active compound with an 8R-carboxamide configuration and thus to the desired C8 epimer. The second and invariably more polar compound with blue fluorescence corresponded to the 8S-carboxamide epimer, also known as the iso-compound. This was confirmed by LCMS for masses (where an inverse order of elution was observed on reversed-phase column) and by NMR for structural proof, by comparing the isolated lysergic acid derivatives with the isolated iso-lysergic acid derivatives as well as with thorough existing literature, e.g., (Brandt et al., 2017; Hoehn et al., 2017; Hoffman & Nichols, 1985; Stachulski et al., 1996). Since not all lysergic acid derivatives separate well from their iso-lysergic acid derivatives on HPLC conditions the inventors did not solely rely on TLC analysis (where, in rare cases, not a distinct separation occurred), ¹H-NMR analysis was used for the proper epimer separation check as well. Generally, there is a hindered rotation about the amide CN bound which can cause more complex spectra. It is important to note, that, for the H9 proton, ¹H-NMR revealed usually only a single signal (usually around 6.3-6.4 ppm, singlet up to multiplet) for symmetrically substituted amides. Most non-symmetrically substituted amides showed two signals (usually around 6.3-6.4 ppm, singlets up to multiplets in some cases) for this H9 proton, in a non 1:1 ratio. To proof the absence of any 8S-configured impurities (iso-compounds) potentially contaminating the desired 8R-configured derivatives—in addition to HPLC UV and MS analysis wherein the 8R/8S epimers were not always baseline separated—the iso-compounds were also measured in ¹H-NMR, and a comparison of spectra indicated also two signals between around 6.3 and 6.4 ppm in a non-1:1 ratio for the H9 proton but both signals having different chemical shifts than the two signals of H9 from the 8R-configured compounds. With this, the epimeric purities were ultimately proven for asymmetric amides as well. The desired free base products were dried under high vacuum to get rid of any residual NH₃ or NEt₃, whereafter a solid or a foam with an aspect of golden, brownish, or beige color was obtained. The obtained iso-compounds (isolated compounds with an 8S-carboxamide configuration) are worth to isolate as well and can easily be epimerized at the C8 center, to get mixtures of 8R- and 8S-carboxamides, by the application of bases in an appropriate solvent by known procedures (GB579484A). The obtained epimeric mixtures are then separated by the above purification steps. By this, yields of lysergic acid derivatives with a desired 8R-carboxamide configuration are easily increased. On larger batches, it is worth to repeat this procedure several times, to maximize yields.

General procedure for hemitartrate or tartrate salt formation. Note: only for the desired 8R-carboxamide epimers conversion to their salts is described, and the C8 epimers (with an 8S configuration, so-called iso-compounds) were either kept as their free bases, or, for ¹H-NMR spectra comparison, some were converted to the salts as well. A solution of 10% (+)-tartaric acid in methanol anhydr. was prepared (exact weighing for calculation of the volumes needed). The purified lysergic acid amide derivative as free base was dissolved in a minimal amount of MeOH anhydr. under slight warming, where necessary, and was neutralized with 0.5 (for hemitartrates) to 1 mol. equivalent (for tartrates) of the above (+)-tartaric acid solution. Alternatively, neutralization could be performed by direct addition of the (+)-tartaric acid solution to the free base foam. Next, diethyl ether (Et₂O) anhydr. was added until the maximum of precipitation was reached. The suspension formed was allowed to stand for the time needed either at ambient temperature or in the fridge, depending on ease of suspension forming. The liquid layer was cautiously decanted or removed by using a front-clogged Pasteur pipet (cotton wool or alike) and the residue was rinsed with MeOH/Et₂O 1:1 and finally with Et₂O before it was dried in high vacuo overnight. The aspects of the residual crystalline lysergic acid amide hemitartrates or tartrates was of white to off-white color. Determination of exact tartaric acid content can be performed by ¹H-NMR (ratio of lysergic acid derivative to tartaric acid: this can be 1:0.5 up to 1:1, or even 1 to more than 1, when an excess of tartaric acid was used incautious, and the excess was not properly removed). Comment on ¹H-NMR spectra of the tartrate salts (see also existing literature, e.g., (Bailey et al., 1973; Hoehn et al., 2017; Hoffman & Nichols, 1985; Stachulski et al., 1996): as observed on the free bases of the lysergic acid derivatives, for the H9 proton ¹H-NMR revealed only a single signal (around 6.3-6.4 ppm, singlet up to multiplet in some cases) for symmetrically substituted amides. Non-symmetrically substituted amides showed two signals (around 6.3-6.4 ppm, singlets up to multiplets in some cases) for this H9 proton, in a non 1:1 ratio. To proof the absence of any 8S-configured impurities (iso-compounds)—in addition to HPLC UV and MS analysis wherein the 8R/8S epimers were not always baseline separated—the iso-compounds were also measured in ¹H-NMR, and a comparison of spectra indicated also a non 1:1 ratio for the H9 proton but having different chemical shifts than the two signals of H9 from the 8R-configured compounds. With this, the epimeric purities were ultimately proven for asymmetric amides as well.

General procedure for N6 alkylations with alkyl halides, adapted from (Hoffman & Nichols, 1985). To a mixture of 97 μmol (in case of 6-Nor-LSD this corresponds to 30 mg) of free base of the N6 Nor-compound and 26.8 mg (2.0 eq) K₂CO₃ in 0.5 mL DMF anhydr. was added 116 μmol (1.3 eq) of the corresponding alkyl halide under Nitrogen. After the reaction control (either TLC: DCM/MeOH 9/1 by, in some cases adding 0.1% NEt₃, or by HPLC UV and MS) indicated complete conversion (note: N6 alkylation is much faster than C8 epimerization and thus, at ambient temperature and under the chosen reaction conditions virtually no epimerization took place, as has been previously demonstrated (Stachulski et al., 1996) the mixture was worked up. In cases where the N6 alkylation was very slow (e.g., less than some 20% conversion after one day) the reactions could significantly be forced towards completion by adding a second, and, in some cases, a third equivalent of alkylating agent after one, two and three days, respectively. Thus, after stirring for the time needed the reaction mixture was concentrated in high vacuo at 40° C. to remove most of the volatiles including DMF and the residue was dissolved in the eluent used for chromatography and purified by silica gel column chromatography as described in the chapter General. For most compounds, a solvent system of DCM/MeOH/NEt₃=98/2/0.1 was suitable. The free base product was dried under high vacuum to get rid of any residual NH₃ or NEt₃, whereafter a solid or a foam with an aspect of golden, brownish, or beige color was obtained. Where necessary, the products could further be purified by dissolution in a hot solvent such as benzene and, when needed, filtering and then precipitating them from the filtered and cooled solution by adding some heptane (Hoffman & Nichols, 1985). A such precipitate is then collected by filtration and dried under high vacuum. When desired, the free base compounds can be converted to a pharmaceutically acceptable salt. In some cases, the inventors observed a very weak salt character, and it was possible, in the case of some tartrate salts, even to separate the tartaric acid completely from the lysergic acid amide derivative by precipitations of the tartaric acid from a solution.

### Examples—Amide Coupling of Lysergic Acid with Secondary Amines and Conversion to their Salts: Preparation of the Lysergic Acid Derivatives 2a-l

9,10-Didehydro-N-methyl-N-propyn-3-yl-6-methylergoline-8R-carboxamide (TRALA-01), 2a. According to the general amide coupling method described, from 286 mg lysergic acid monohydrate (A. T. Shulgin & Shulgin, 1997), 258 mg CDI and 276 mg N-methyl-propargylamine (4 eq), no DIPEA used. Yield: 77 mg (24%) TRALA-01 as a beige amorphous solid and 75 mg (18%) iso-TRALA-01 as a gray-greenish solid. Tartrate salt formation according to the general method described; yield: 62 mg 2a tartrate product as an off-white solid. Analytical data of 2a as tartrate: ¹H-NMR (DMSO-d6): (relating complexity of interpretation: see chapter General; amide couplings) ˜2.50 (s, N(6)Me, superimposed by DMSO), 2.59 (m, 1H), 2.92 (s, 1H), 3.11 (m, 2H), 3.23 (t, 1H), 3.25-3.44 (m, ˜2H), 3.51 (m, ˜2H), 3.89-4.38 (m, ˜3H), 4.23 (s, tartaric acid), 6.28 (ca. 60%)/6.35 (ca. 40%) (2×s, sum=H9; note; epimeric purity proof of C9 of the ergoline structure: see chapter General; amide couplings), 7.00-7.11 (m, 3 arom. H), 7.17-7.23 (m, 1 arom. H), 10.75 (bs, NH). LCMS (M+H): expected for 2a: M=319.41; found: 320.3.

9,10-Didehydro-N-ethyl-N-propyn-3-yl-6-methylergoline-8R-carboxamide (TRALA-02), 2b. According to the general amide coupling method described, from 286 mg lysergic acid monohydrate, 258 mg CDI, 180 mg N-ethyl-propargylamine hydrochloride (1.5 eq) and 435 μL DIPEA (2.5 eq). Yield: 110 mg (33%) TRALA-02 as a brownish amorphous solid and 116 mg (35%) iso-TRALA-02 as a brown solid. Tartrate salt formation according to the general method described; yield: 104 mg product 2b tartrate as an off-white solid. Analytical data of 2b as tartrate: ¹H-NMR (DMSO-d6): (relating complexity of interpretation: see chapter General; amide couplings) 1.11/1.25 (2×t, sum=3H), 2.54 (s, N(6)Me, superimposed by DMSO), 2.69 (t, ˜1H), 3.04-3.21 (m, ˜3H), 3.35-3.65 (m, ˜4H), 3.96 (m, ˜1H), 4.21 (m, ˜1H), 4.24 (s, tartaric acid), 4.35 (m, 1H), 6.25 (ca. 50%)/6.36 (ca. 50%) (2×s, sum=H9; note; epimeric purity proof of C9 of the ergoline structure: see chapter General; amide couplings), 7.00-7.11 (m, 3 arom. H), 7.17-7.23 (m, 1 arom. H), 10.74 (bs, NH). LCMS (M+H): expected for 2b: M=333.43; found: 334.2. Analytical data of iso-2b as tartrate: ¹H-NMR (DMSO-d6): 1.10/1.26 (2×t, sum=3H), 2.64 (s, N(6)Me), 2.76 (t, ˜1H), 2.98 (m, ˜1H), 3.13 (m, ˜1H), 3.27-3.67 (m, ˜4H), 3.87 (m, ˜1H), 4.16 (m, ˜1H), 4.20 (s, tartaric acid), 4.39 (m, 1H), 6.32 (ca. 55%)/6.39 (ca. 45%) (2×s, sum=H9; note; epimeric purity proof of C9 of the ergoline structure: see chapter General; amide couplings), 7.07 (bs, 3 arom. H), 7.14-7.23 (m, 1 arom. H), 10.76 (bs, NH). LCMS (M+H): expected for iso-2b: M=333.43; found: 334.2.

9,10-Didehydro-N-(cyanomethyl)-N-ethyl-6-methylergoline-8R-carboxamide (TRALA-03), 2c. According to the general amide coupling method described, from 286 mg lysergic acid monohydrate, 258 mg CDI and 168 mg 2-(ethylamino)acetonitrile (2 eq), no DIPEA used. Yield: 30 mg (9%) TRALA-03 as a beige amorphous solid and 104 mg (31%) iso-TRALA-03 as a brownish mass. Tartrate salt formation according to the general method described; yield: 31 mg product 2c tartrate as an off-white solid. Analytical data of 2c as tartrate: ¹H-NMR (DMSO-d6): (relating complexity of interpretation: see chapter General; amide couplings) 1.13/1.26 (2×t, sum=3H), 2.52 (m, ˜1H, superimposed by DMSO), 2.56 (s, N(6)Me, superimposed by DMSO), 2.71 (t, ˜1H), 3.15 (m, ˜2H), 3.59 (m, ˜3H), 3.96 (m, ˜1H), 4.27 (s, tartaric acid), 4.41 (m, ˜2H), 6.28 (s, H9), 7.03-7.10 (m, 3 arom. H), 7.19-7.24 (m, 1 arom. H), 10.74 (bs, NH). LCMS (M+H): expected for 2c: M=334.42; found: 335.2.

9,10-Didehydro-N-ethyl-N-(2-fluoroethyl)-6-methylergoline-8R-carboxamide (TRALA-04), 2d. According to the general amide coupling method described, from 573 mg lysergic acid monohydrate, 517 mg CDI, 255 mg N-ethyl-(2-fluoroethyl)amine hydrochloride (1 eq) and 523 μL DIPEA (1.5 eq). Yield: 153 mg (22%) TRALA-04 as a beige foam and 156 mg (23%) iso-TRALA-04 as a brown foam. Tartrate salt formation according to the general method described; yield: 158 mg 2d tartrate as an off-white solid. Analytical data of 2d as tartrate: ¹H-NMR (DMSO-d6): (relating complexity of interpretation: see chapter General; amide couplings) 1.07/1.21 (2×t, sum=3H), 2.52 (m, ˜1H, superimposed by DMSO), 2.54 (s, N(6)Me, superimposed by DMSO), 2.69 (t, 1H), 3.01-3.19 (m, ˜2.5H), 3.41 (m, ˜1H), 3.53 (m, ˜2.5H), 3.62-3.95 (m, ˜2.5H), 4.23 (s, tartaric acid), 4.59 (t×q, 2H), 6.27 (s, H9), 7.00-7.11 (m, 3 arom. H), 7.17-7.23 (m, 1 arom. H), 10.73 (bs, NH). ¹⁹F-NMR (DMSO-d6): −221.29, −222.13. LCMS (M+H): expected for 2d: M=341.43; found: 342.3.

9,10-Didehydro-N-(2,2-difluoroethyl)-N-ethyl-6-methylergoline-8R-carboxamide (TRALA-05), 2e. According to the general amide coupling method described, from 492 mg lysergic acid monohydrate, 444 mg CDI, 250 mg N-(2,2-difluoroethyl)ethylamine hydrochloride (1 eq) and 448 μL DIPEA (1.5 eq). Yield: 202 mg (33%) TRALA-05 as a golden foam and 252 mg (41%) iso-TRALA-05 as a brown mass. Tartrate salt formation according to the general method described; yield: 204 mg 2e tartrate as an off-white solid. Analytical data of 2e as tartrate: ¹H-NMR (DMSO-d6): (relating complexity of interpretation: see chapter General; amide couplings) 1.08/1.23 (2×t, sum=3H), 2.53 (s, N(6)Me, superimposed by DMSO), 2.62 (m, ˜1.5H, superimposed by DMSO/N(6)Me), 3.02-3.19 (m, ˜2.5H), 3.41 (m, ˜1H), 3.54 (m, ˜2.5H), 3.74 (t×m, 1.5H), 3.93 (m, 1.5H), 4.25 (s, tartaric acid), 6.15 (t×m, 1H), 6.24 (minor)/6.26 (major) (2×s, sum=H9; note; epimeric purity proof of C9 of the ergoline structure: see chapter General; amide couplings), 7.00-7.11 (m, 3 arom. H), 7.17-7.24 (m, 1 arom. H), 10.73 (bs, NH). ¹⁹F-NMR (DMSO-d6): −120.46 (major), −122.04 (minor). LCMS (M+H): expected for 2e: M=359.42; found: 360.3. Analytical data of iso-2e as tartrate: ¹H-NMR (DMSO-d6): (relating complexity of interpretation: see chapter General; amide couplings) 1.05/1.23 (2×t, sum=3H), 2.64 (m, N(6)Me), 2.77 (m, 1H), 2.97 (m, 1H), 3.13 (m, 1H), 3.32 (m, ˜2H), 3.48-3.78 (m, ˜4H), 3.81-4.10 (m, ˜1.8H), 4.22 (s, tartaric acid), 6.11 (t×m, 1H), 6.30 (s, H9; note; epimeric purity proof of C9 of the ergoline structure: see chapter General; amide couplings), 7.07 (m, 3 arom. H), 7.21 (m, 1 arom. H), 10.76 (bs, NH). ¹⁹F-NMR (DMSO-d6): −120.42 (major), −121.87 (minor). LCMS (M+H): expected for iso-2e: M=359.42; found: 360.3.

9,10-Didehydro-N-ethyl-N-(2,2,2-trifluoroethyl)-6-methylergoline-8R-carboxamide (TRALA-06), 2f. According to the general amide coupling method described, from 573 mg lysergic acid monohydrate, 517 mg CDI, 328 mg N-(2,2,2-trifluoroethyl)ethylamine hydrochloride (1 eq) and 522 μL DIPEA (1.5 eq). Yield: 128 mg (17%) TRALA-06 as a yellowish foam and 170 mg (23%) iso-TRALA-06 as a brown mass. Tartrate salt formation according to the general method described; yield: 108 mg 2f tartrate as an off-white solid. Analytical data of 2f as tartrate: ¹H-NMR (DMSO-d6): (relating complexity of interpretation: see chapter General; amide couplings) 1.09/1.23 (2×t, sum=3H), 2.54 (s, N(6)Me, superimposed by DMSO), 2.67 (m, ˜1H, superimposed by DMSO/N(6)Me), 2.99-3.20 (m, ˜3H), 3.54 (m, ˜3H), 3.96 (m, 1H), 4.24 (m, ˜1.5H, superimposed by tartaric acid), 4.26 (s, tartaric acid), 4.47 (m, ˜0.5H), 6.21 (minor)/6.24 (major) (2×s, sum=H9; note; epimeric purity proof of C9 of the ergoline structure: see chapter General; amide couplings), 6.99-7.12 (m, 3 arom. H), 7.17-7.23 (m, 1 arom. H), 10.74 (bs, NH). ¹⁹F-NMR (DMSO-d6): −68.27 (major), −69.38 (minor). LCMS (M+H): expected for 2f: M=377.41; found: 378.3.

9,10-Didehydro-N-methyl-N-(2,2,2-trifluoroethyl)-6-methylergoline-8R-carboxamide (TRALA-07), 2g. According to the general amide coupling method described, from 573 mg lysergic acid monohydrate, 517 mg CDI, 299 mg N-(2,2,2-trifluoroethyl)methylamine hydrochloride (1 eq) and 522 μL DIPEA (1.5 eq). Yield: 108 mg (15%) TRALA-07 as a yellow foam and 174 mg (24%) iso-TRALA-07 as a brown foam. Tartrate salt formation according to the general method described; yield: 86 mg 2g tartrate as an off-white solid. Analytical data of 2g as tartrate: ¹H-NMR (DMSO-d6): (relating complexity of interpretation: see chapter General; amide couplings) 2.54 (s, N(6)Me, superimposed by DMSO), 2.63 (m, ˜1.5H, superimposed by DMSO/N(6)Me), 2.97-3.20 (m, 3H), 3.28 (s, CONMe), 3.52 (d×d, 1H), 4.15 (m, 1H), 4.24 (m, 2H), 4.26 (s, tartaric acid), 4.53 (m, ˜0.5H), 4.69 (d×t, 2H), 6.23 (minor)/6.29 (major) (2×s, sum=H9; note; epimeric purity proof of C9 of the ergoline structure: see chapter General; amide couplings), 7.01-7.12 (m, 3 arom. H), 7.17-7.23 (m, 1 arom. H), 10.73 (bs, NH). ¹⁹F-NMR (DMSO-d6): −68.62 (major), −69.36 (minor). LCMS (M+H): expected for 2g: M=363.39; found: 364.3. Analytical data of iso-2g as tartrate: ¹H-NMR (DMSO-d6): (relating complexity of interpretation: see chapter General; amide couplings) 2.61 (s, N(6)Me), 2.73 (t, 1H), 2.94 (m, ˜1.6H), 3.08 (d×d, ˜1.3H), 3.29 (s, CONMe), 3.42 (m, ˜1H), 3.94 (m, 1H), 4.21 (m, ˜2H, superimposed from tartaric acid), 4.26 (s, tartaric acid), 6.27 (minor)/6.33 (major) (2×s, sum=H9; note; epimeric purity proof of C9 of the ergoline structure: see chapter General; amide couplings), 7.01-7.12 (m, 3 arom. H), 7.16-7.24 (m, 1 arom. H), 10.75 (bs, NH). ¹⁹F-NMR (DMSO-d6): −68.52 (major), −69.13 (minor). LCMS (M+H): expected for iso-2g: M=363.39; found: 364.3.

9,10-Didehydro-N,N-di(2-fluoroethyl)-6-methylergoline-8R-carboxamide (TRALA-08), 2h. According to the general amide coupling method described, from 492 mg lysergic acid monohydrate, 444 mg CDI, 250 mg di(2-fluoroethyl)amine hydrochloride (1 eq) and 448 μL DIPEA (1.5 eq). Yield: 80 mg (13%) TRALA-08 as a beige foam and 161 mg (26%) iso-TRALA-08 as a beige foam. Tartrate salt formation according to the general method described; yield: 76 mg 2h tartrate as an off-white solid. Analytical data of 2h as tartrate: ¹H-NMR (DMSO-d6): 2.57 (s, NMe, superimposed by DMSO), 2.68 (t, 1H), 3.03-3.21 (m, 3H), 3.52 (d×d, 1H), 3.70 (d×m, 2H), 3.87 (d×t, 2H), 3.98 (m, 1H), 4.24 (s, tartaric acid), 4.53 (d×t, 2H), 4.69 (d×t, 2H), 6.28 (s, H9), 7.01-7.12 (m, 3 arom. H), 7.18-7.23 (m, 1 arom. H), 10.73 (bs, NH). ¹⁹F-NMR (DMSO-d6): −221.98, −222.95. LCMS (M+H): expected for 2h: M=359.42; found: 360.3.

9,10-Didehydro-N,N-bis(2,2-difluoroethyl)-6-methylergoline-8R-carboxamide (TRALA-09), 2i. According to the general amide coupling method described, from 394 mg lysergic acid monohydrate, 356 mg CDI, 250 mg bis(2,2-difluoroethyl)amine hydrochloride (1 eq) and 360 μL DIPEA (1.5 eq). Yield: 33 mg (6%) TRALA-09 as a brown mass and 44 mg (8%) iso-TRALA-09 as a brown mass. Tartrate salt formation according to the general method described; yield: 18 mg 2i tartrate as an off-white solid. Analytical data of 2i as tartrate: ¹H-NMR (DMSO-d6): (relating complexity of interpretation: see chapter General; amide couplings) ˜2.50 (NMe, superimposed by DMSO), 2.64 (t, 1H), 3.03-3.28 (m, 3H), 3.52 (d×d, 1H), 3.86 (txt, 2H), 3.97-4.15 (m, 3H), 4.27 (s, tartaric acid), 6.24 (s, H9), 6.25 (5.97-6.53: sharply split t×m; 2×CHF₂), 7.02-7.14 (m, 3 arom. H), 7.18-7.25 (m, 1 arom. H), 10.74 (bs, NH). ¹⁹F-NMR (DMSO-d6): −121.26, −121.29, −122.87. LCMS (M+H): expected for 2i: M=395.40; found: 396.2.

9,10-Didehydro-N-ethyl-N-(methoxy)-6-methylergoline-8R-carboxamide (TRALA-10), 2j. According to the general amide coupling method described, from 1.14g lysergic acid monohydrate, 1.04g CDI, 0.51 g N-methoxy-ethylamine hydrochloride (1 eq) and 1.04 mL DIPEA (1.5 eq). Yield: 113 mg (17%) TRALA-10 as a yellow foam and 146 mg (22%) iso-TRALA-10 as a brown foam. Tartrate salt formation according to the general method described; yield: 74 mg 2j tartrate as an off-white solid. Analytical data of 2j as tartrate: ¹H-NMR (DMSO-d6): (relating complexity of interpretation: see chapter General; amide couplings): 1.13 (t, 3H), 2.56 (s, NMe, superimposed by DMSO), 2.62 (m, ˜1H), 3.06-3.18 (m, 3H), 3.52 (d×d, 2H), 3.66 (q, 2H), 3.76 (s, OMe), 3.93 (m, 1H), 4.25 (s, tartaric acid), 6.30 (s, H9), 7.01-7.12 (m, 3 arom. H), 7.18-7.23 (m, 1 arom. H), 10.73 (bs, NH). LCMS (M+H): expected for 2j: M=325.41; found: 326.3.

9,10-Didehydro-6-methylergoline-8R-((RS)-2-ethynylazetidide) (TRALA-11), 2k. According to the general amide coupling method described, from 243 mg lysergic acid monohydrate, 220 mg CDI, 100 mg (RS)-2-ethynylazetidine hydrochloride (1 eq) and 222 μL DIPEA (1.5 eq). Yield: 52 mg (19%) TRALA-11 as a beige foam and 90 mg (32%) iso-TRALA-11 as a beige foam. Tartrate salt formation according to the general method described; yield: 45 mg 2k tartrate as an off-white solid. Analytical data of 2k as tartrate (relating complexity of interpretation: see chapter General; amide couplings): ¹H-NMR (DMSO-d6): 2.24 (m, 1H), 2.48-2.62 (m, ca. 3H, superimposed by DMSO and NMe), 2.52 (s, NMe, superimposed by DMSO), 3.12 (m, 1H), 3.18 (s, CCH), 3.52 (m, 2H), 3.75 (m, 1H), 3.86 (m, ca. 1.5H), 4.25 (s, tartaric acid), 4.27 (m, ca. 0.5H), 4.83 (m, ca. 0.5H), 4.32 (m, ca. 0.5H), 6.26 (minor)/6.35 (major) (2×s, sum=H9; note: the azetidine moiety contains a stereocenter which is assumed to be racemic; epimeric purity proof of C9 of the ergoline structure: see chapter General; amide couplings) 7.01-7.11 (m, 3 arom. H), 7.21 (m, 1 arom. H), 10.73 (bs, NH). LCMS (M+H): expected for 2k: M=331.42; found: 332.2.

9,10-Didehydro-N-(2-fluoroethyl)-N-(methoxy)-6-methylergoline-8R-carboxamide (TRALA-14), 2n. 1.) Preparation of N-methoxy-2-fluoroethylamine hydrochloride (10; adapted from US20100029670A1). To a solution of 1.0g (8.39 mmol) ethyl N-methoxycarbamate (8) in 5 mL DMF anhydr. were added 0.352g (8.81 mmol) NaH 60% dispersed in mineral oil under nitrogen and ice-cooling. After stirring for 5 min the mixture was allowed to stir at ambient temperature for 1h. Next, 1.46g (8.39 mmol) 1-fluoro-2-iodomethane was added and the mixture was heated to 75° C. for 6h. The mixture was cooled to ambient temperature and mixed with water and EtOAc (70 mL, each), and the layer were separated. The org. layer was further washed once with water (1×70 mL), dried over MgSO₄, and concentrated in vacuo to get 1.05g (75%) of the intermediate 9 as a yellow oil. ¹H-NMR (CDCl₃): 1.33 (t, CH2CH₃), 3.75 (s, OCH₃), 3.81 (d×t, CH2CH2F), 4.24 (q, CH2CH3), 4.59 (d×t, CH2F). ¹⁹F-NMR (CDCl₃): −224.0. The intermediate 9 (1.03g) was mixed with 1.5 mL EtOH, 1.5 mL water and with 1.25g KOH, and the mixture was heated to 75° C. for 5h, whereby the flask (25 mL) was plugged with a septum attached to a Teflon tube. The second end of the tube was placed into a small gas washing bottle containing 2M aq. HCl. After the reaction time the mixture was heated to 90° C. and any residual product was forced to transfer to the gas washing bottle using a slow Nitrogen stream (balloon, needle, over 1 h). The 2M HCl containing the product was concentrated in vacuo, and the residual semi-solid was co-evaporated with MeOH, quickly dried in high-vacuo and then triturated with Et₂₀/hexane and filtered off. After drying there were obtained 326 mg (40%) N-methoxy-2-fluoroethylamine hydrochloride (10) as a rose-colored solid. ¹H-NMR (soluble in CDCl₃): 3.66 (d×t, CH2CH2F) 4.12 (s, OCH₃), 4.89 (d×t, CH2F). ¹⁹F-NMR (CDCl₃): −223.2.2.) Amide coupling reaction: According to the general amide coupling method described, from 248 mg lysergic acid monohydrate, 155 mg (1.1 eq) CDI, 123.5 mg N-methoxy-2-fluoroethylamine hydrochloride (10; 1.1 eq) and 377 □L DIPEA (2.5 eq); the solution of amine 10 and DIPEA in DMF was added dropwise under ice-cooling, and the reaction mixture was allowed to warm to ambient temperature over several hours. Yield: 21 mg (7%) TRALA-14 as a golden beige solid. Analytical data of 2n: ¹H-NMR (CDCl₃): 2.62 (s, NMe), 3.24 (m, 2H), 3.57 (d×d, 1H), 3.81 (s, OMe), 3.95 (d×t, 1H), 4.04 (d×t, 1H), 4.10 (bm, 1H), 4.24 (m, 1H), 4.57 (t, 1H), 4.73 (t, 1H), 6.47 (s, H9) 6.92 (m, 1 arom. H), 7.14-7.26 (m, 3 arom. H), 7.55 (m, 0.5H), 7.73 (m, 0.5H), 7.98 (bs, NH). ¹⁹F-NMR (CDCl₃): −222.5. LCMS (M+H): expected for 2n: M=343.40; found: 344.2.

### Examples—Enamides of Lysergic Acid, Preparation of Derivatives 21-m

1.) Amide Formation

9,10-Didehydro-N-ethyl-N-(2-(phenylthio)ethyl)-6-methylergoline-8R-carboxamide, 4. According to the general amide coupling method described, from 974 mg lysergic acid monohydrate, 880 mg CDI, 885 μL N-ethyl-2-(phenylthiol)ethanamine (3; 1.1 eq) and 885 μL DIPEA (1.5 eq). Yield: 550 mg (38%) title product as a golden foam. Analytical data of 4: ¹H-NMR (CDCl₃): 1.12-1.32 (m, 3H), 2.59-2.78 (m, 4H), 2.92 (t, 1H), 3.04 (m, 1H), 3.10-3.32 (m, 3H), 3.40-3.68 (m, 5H), 3.89 (bm, 1H), 6.30/6.37 (2×s, H9) 6.93 (t, 1 arom. H), 7.15-7.49 (m, 8 arom. H), 8.00 (bs, NH). LCMS (M+H): expected for 4: M=431.60; found: 432.3.

2.) Sulfoxide Formation

9,10-Didehydro-N-ethyl-N-(2-(phenylsulfinyl)ethyl)-6-methylergoline-8R-carboxamide, 5. To an ice-cooled solution of 405 mg (0.94 mmol) 4 in 20 mL DCM were added 81 μL aq. HCl 37% (1.05 eq). Next, a solution of 211 mg (1.0 eq) meta-chloroperbenzoic acid (mCPBA) in 20 mL DCM was added over the course of 5 min. After 20 min, LCMS analysis indicated formation of sulfoxide (50%; a small second peak having the same mass corresponded to the N-oxide, identities proven by isolation and ¹H-NMR), as well as double oxidated product (20%), among starting material (30%), and the reaction was quenched by the addition of 20 mL of aq. 10% Na₂S₂O₃ solution. After stirring vigorously for 5 min, the layers were separated and the aq. layer was further extracted with DCM (2×20 mL), and the combined org. layers were dried over Na₂SO₄ and concentrated in vacuo. The greenish-black residue (547 mg) was purified by silica gel chromatography (DCM/MeOH/NEt₃). Yield: 54 mg (13%) title product as an off-white foam, among 258 mg recovered starting material. Analytical data of 5: ¹H-NMR (CDCl₃; note: the sulfoxide bears an additional chiral center and adds complexity): 1.15-1.36 (m, 3H), 2.58-2.78 (m, 4H), 2.80-2.95 (bm, 1H), 2.95-3.20 (m, ˜2.5H), 3.25-3.38 (m, ˜2.5H), 3.5-3.8 (m, 5H), 3.93 (bm, 1H), 6.23/6.27 (both minor) and 6.32/6.38 (both major) (each as a s, H9; sum=1H) 6.94 (m, 1 arom. H), 7.15-7.27 (m, 3 arom. H), 7.50-7.61 (m, 3 arom. H), 7.65-7.72 (m, 2 arom. H), 7.95 (bs, NH). LCMS (M+H): expected for 5: M=447.60; found: 448.2.

3.) Thermolysis

9,10-Didehydro-N-ethenyl-N-ethyl-6-methylergoline-8R-carboxamide (TRALA-12), 21. A mixture of 54 mg (0.121 mmol) 9,10-Didehydro-N-ethyl-N-(2-(phenylsulfinyl)ethyl)-6-methylergoline-8R-carboxamide (5) and 51 mg (5 eq.) NaHCO₃ in 8 mL m-xylene was heated to 130-140° C. under nitrogen. After a total of 2 days there were observed some 25% conversion (LCMS), among starting material and some decomposition products. The volatiles were removed in high vacuo at 50° C. and the residue was purified by first dissolving it in 1 mL DCM/MeOH/NEt₃=90/10/0.2 and filtering it through a silica gel pad (height 1 cm) using 70 mL of the same solvent system. This removed most of the dark color. The eluate was concentrated in vacuo and the residue was further purified by silica gel chromatography using the same solvent system starting at 98% DCM. There were obtained ca. 2 mg of the title product. Analysis on either basic or acidic HPLC MS (see chapter General) indicated the same purity (approx. 85%) revealing the product's stability against these conditions. LCMS (M+H): expected for 21: M=321.43; found: 322.2. ¹H-NMR (CDCl₃): the signals were in accordance with the spectrum obtained by the alternative route (e.g., 6 to 7 to 5 to 21; FIG. 9; LCMS retention times also in accordance) but the ¹H-NMR also indicated significant impurities herein.

4.) Enamides by Base-Promoted Elimination Reactions (Microscale)

9,10-Didehydro-N-(2,2-difluoroethenyl)-N-ethyl-6-methylergoline-8R-carboxamide (TRALA-13), 2m. This reaction could be performed either with the use of LDA or with BuLi, no epimerization observed. A solution of 7 mg (18.5 μmol) TRALA-06 (2f) in 0.2 mL THF anhydr. under nitrogen was cooled to −100° C. (liquid nitrogen, acetone/THF 4:1 mixture as cooling bath). Next, 2.0 eq of 1.6M BuLi or, in a second experiment, 2.0 eq. of a freshly prepared lithium diisopropylamide solution in THF (from BuLi and diisopropylamine in THF) were added within 30 s. Both basic and acidic HPLC MS from a sample hydrolyzed in a drop water and diluted with MeOH indicated 30% product formation (according to integral of UV absorption at 313 nm, as well as according to integral of e/z=358, versus the starting material) among intact starting material; there was no significant decomposition observed and the product remained intact. To further test chemical stability, a hydrolyzed sample stored at ambient temperature overnight (thus, basic conditions) remained intact. Another hydrolyzed sample was made acidic by addition of excess tartaric acid and, after storing for 24h, reanalysis indicated the same product distribution as after initial hydrolysis of the reaction. LCMS (M+H): expected for 2m: M=357.41; found: 358.3.

### Examples—Alternative Route to Enamides of Lysergic Acid: Preparation of Derivative 2l

1.) Hydroamination

N-ethyl-2-(phenylsulfinyl)ethanamine, 7 The procedure was adapted from (Hu et al., 2014). To ethylamine 2M in THF anh. (6 mL; 12 mmol) was added 1.33 mL (10 mmol) phenylvinyl sulfoxide (6) and the clear solution was allowed to stir for 18h under nitrogen. The volatiles were removed in vacuo at 50° C. and the residual viscous orangish oil was purified by silica gel chromatography (DCM/MeOH/NEt_{3=100/0/0.5} to 95/5/0.5). Yield: 1.72g (72%) title product as a colorless oil. Analytical data of 7: ¹H-NMR (CDCl₃; note: the sulfoxide bears a chiral center; enantiomeric ratio not determined): 1.11 (t, CH3), 2.67 (q, NHCH₂), 2.96 (m, CH2), 3.13 (m, CH2), 7.48-7.57 (m, 3 arom. H), 7.62-7.67 (m, 2 arom. H). LCMS (M+H): expected for 7: 197.30; found: 198.1.

2.) Amide Formation

9,10-Didehydro-N-ethyl-N-(2-(phenysulfinyl)ethyl)-6-methylergoline-8R-carboxamide, 5. According to the general amide coupling method described, from 394 mg lysergic acid monohydrate, 356 mg CDI, 272 mg N-ethyl-2-(phenylsulfinyl)ethanamine (7; 1.0 eq) and 356 μL DIPEA (1.5 eq). Yield: 191 mg (31%) title product as a beige solid. Analytical data of 5: ¹H-NMR (CDCl₃; note: the sulfoxide bears an additional chiral center and adds complexity; due to its synthesis path, herein it might rather be racemic): 1.15-1.36 (m, 3H), 2.58-2.78 (m, 4H), 2.80-2.95 (bm, 1H), 2.95-3.20 (m, ˜2.5H), 3.25-3.38 (m, ˜2.5H), 3.5-3.8 (m, 5H), 3.93 (bm, 1H), 6.23/6.27 (both minor; first more dominant) and 6.32/6.38 (both major) (H9; sum=1H) 6.92/6.94 (m, 1 arom. H), 7.15-7.27 (m, 3 arom. H), 7.50-7.61 (m, 3 arom. H), 7.65-7.72 (m, 2 arom. H), 7.95 (bs, NH). LCMS (note: the same retention time obtained as for the sulfoxide 5 obtained via the alternative route, see chapter “2.) Sulfoxide formation;” M+H): expected for 5: 447.60; found: 448.2.

3.) Thermolysis

9,10-Didehydro-N-ethenyl-N-ethyl-6-methylergoline-8R-carboxamide (TRALA-12), 21. A mixture of 180 mg (0.402 mmol) 9,10-Didehydro-N-ethyl-N-(2-(phenylsulfinyl)ethyl)-6-methylergoline-8R-carboxamide (5) and 283 mg (5 eq.) K₂CO₃ in 26 mL m-xylene was heated to 130-140° C. under nitrogen. After a total of 22 hours there were observed some 40% conversion (LCMS), among starting material and only minor decomposition products. Longer heating provoked progressive decomposition. The volatiles were removed in high vacuo at 50° C. and the residue was purified by first dissolving it in 5 mL DCM/MeOH/NEt₃=90/10/0.1 and filtering it through a silica gel pad (height 1 cm) using 150 mL of the same solvent system. This removed most of the dark color. The eluate was concentrated i.v. and the residue was further purified by silica gel chromatography using DCM/MeOH/NEt₃=98/2/0.1 to 90/10/0.1 as eluent. The crude product (24 mg) eluted first (and second the starting material; recovered: 89 mg) and was further purified by silica gel prep. TLC using DCM/MeOH/NEt₃=98/2/0.1 as eluent. Finally, there were obtained 9 mg (7%) of the title product 21. Analysis on either basic or acidic HPLC MS indicated the same purity (approx. 90%) revealing the product's stability against these conditions. ¹H-NMR (CDCl₃): (relating complexity of interpretation: see chapter General; amide couplings) 1.3 (m; superimposed by some Et₂O and impurities, CH2CH3), 2.63 (s, NMe), 2.74 (t×m, 1H), 2.87 (m, 1H), 3.16 (d×d, 1H), 3.28 (bm, 1H), 3.56 (d×d, 1H), 3.78 (m, 2H), 4.10 (bm, 1H), 4.43 (d, 1H), 4.62 (d, 1H), 6.34-6.44 (two superimposed s, H9; sum=1H; note: epimeric purity proof of C9 of the ergoline structure: see chapter General; amide couplings) 6.93 (m, 1 arom. H), 7.03 (d×d, 1H), 7.15-7.27 (m, 3 arom. H), 7.95 (bs, NH). LCMS (M+H): expected for 21: M=321.43; found: 322.2.

### Examples—N6-Substituted Derivatives of 6-Nor-Lysergic Acid Diethylamide, Preparation of Derivatives 12a-g

1.) Synthesis of LSD

9,10-Didehydro-N,N-diethyl-6-methylergoline-8R-carboxamide (LSD), 2o. According to the general amide coupling method described, from 3.52g lysergic acid monohydrate, 3.18g CDI and 13.4 mL diethylamine (10 eq; no DIPEA used). Yield: 1.64g (41%) LSD as a beige foam and 0.65g (16%) iso-LSD as a brown sticky mass. Analytical data of 20 (LSD) as free base in accordance with lit. ref.: ¹H-NMR (CDCl₃): 1.19 (t, 1×CH₂CH₃), 1.26 (t, 1×CH₂CH₃), 2.63 (s, NMe), 2.72 (t×m, 1H), 2.93 (t, 1H), 3.08 (d×d, 1H), 3.26 (bm, 1H), 3.47 superimposed with 3.57 (m and d×d, total 5H), 3.92 (bm, 1H), 6.37 (s (hint of a triplet), H9. Note: the isolated epimer iso-LSD showed this signal as a tat 6.31), 6.93 (t, 1 arom. H), 7.14-7.27 (m, 3 arom. H), 7.99 (bs, NH). LCMS (M+H): expected for 2o: M=323.41; found: 324.3.

2.) N6-Demethylation of LSD: Preparation of 6-Nor-LSD.

9,10-Didehydro-N,N-diethylergoline-8R-carboxamide (6-Nor-LSD), 11. This has been adapted from (WO2006128658A1). To an ice-cooled solution of 1.35g (4.17 mmol) LSD (20) in 40 mL DCM was added 1.13g (1.2 eq) mCPBA Q77% (wet). After stirring for 10 min (note: LCMS analysis indicated clean and complete formation of the N-oxide intermediate as two chromatographically well separated epimers with e/z=340; reason: the N-oxide group bears an additional chiral center) a freshly prepared solution of 580 mg (0.5 eq) FeSO₄ heptahydrate in 3.0 mL MeOH p.A. was added quickly. The cooling bath was removed and stirring at ambient temperature was continued until complete disappearance of the N-oxides (note: the reason for incomplete conversion of the N-oxide towards the desired product is because one of the N-oxide epimers converts more quickly back to the starting material LSD than N-demethylation rate takes place, see e.g., (McCamley, Ripper, Singer, & Scammells, 2003). By varying reaction conditions to form the N-oxides of, e.g., LSD, the epimeric ratio of N-oxides can be influenced which will, therefore, lead to higher N-demethylation rates; on file, unpublished results). After 3.5h the reaction mixture was poured into 50 mL 0.1 M ethylenediaminetetraacetic acid (EDTA) solution with a pH=9 (adjusted with NH₄OH 25% aq.). After vigorous shaking, the layers were filtered through a small celite pad and then separated, and the aq. layer was further extracted with DCM (3×50 mL). The combined org. layers were dried over Na₂SO₄ and concentrated in vacuo. The dark brown residue was purified by silica gel chromatography (DCM/MeOH/NH₃=95/5/0.1 to 90/10/0.1). There was obtained 449 mg recovered LSD (20; eluted first) and 587 mg (46%) 6-Nor-LSD (11) as a tan solid. The recovered LSD (20) could easily be reused for the same reaction which yielded each time the desired 6-Nor-LSD (11) in essentially the same yields (reaction repeated twice from recovered LSD). ¹H-NMR (CDCl₃): 1.20 (t, 1×CH₂CH₃), 1.30 (t, 1×CH₂CH₃), 2.81 (t×m, 1H), 3.23-3.58 (m, 8H), 3.69 (m, 1H), 3.96 (m, 1H), 6.38 (t, H9), 6.92 (t, 1 arom. H), 7.15-7.26 (m, 3 arom. H), 7.97 (bs, N1H). LCMS (M+H): expected for 11: M=309.41; found: 310.3.

3.) N6-Alkylation of 6-Nor-LSD: Preparation of Compounds 12a-g.

9,10-Didehydro-N,N-diethyl-6-(2-fluoroethyl)ergoline-8R-carboxamide (TRALA-15), 12a. According to the general procedure for N6 alkylation described, from total 28.5 μL (3×9.5 μL, 2^{nd} addition on day 2, 3^{rd} addition on day 3) 1-fluoro-2-iodoethane iodide 30 mg 6-Nor-LSD (11), reaction time: 3 days. Yield: 9 mg (26%) TRALA-15 as a beige foam. Analytical data of 12a as free base: ¹H-NMR (CDCl₃): 1.20 (t, 1×CH₂CH₃), 1.27 (t, 1×CH₂CH₃), 2.73 (t, 1H), 2.85-3.11 (m, 2H), 3.18-3.63 (m, 8H), 3.87 (bm, 1H), 4.68 (d×m, CH2F, 2H), 6.36 (s, H9), 6.91 (t, 1 arom. H), 7.14-7.25 (m, 3 arom. H), 8.04 (bs, NH). ¹⁹F-NMR (CDCl₃): −219.0 (s). LCMS (M+H): expected for 12a: M=355.46; found: 356.3.

9,10-Didehydro-N,N-diethyl-6-(3-fluoropropyl)ergoline-8R-carboxamide (TRALA-16), 12b. According to the general procedure for N6 alkylation described, from total 35.4 μL (2×17.7 μL, 2^{nd} addition on day 2) 1-bromo-3-fluoropropane and 50 mg 6-Nor-LSD (11), reaction time: 2 days. Yield: 12 mg (20%) TRALA-16 as a yellow solid. Analytical data of 12b as free base: ¹H-NMR (CDCl₃): 1.20 (t, 1×CH₂CH₃), 1.27 (t, 1×CH₂CH₃), 1.9-2.1 (bm, 2H), 2.60-2.84 (m, 2H), 2.84-2.98 (m, 2H), 3.14 (bm, 2H), 3.4-3.6 (m, 6H), 3.82 (bm, 1H), 4.61 (d×m, CH2F, 2H), 6.35 (s, H9), 6.93 (s, 1 arom. H), 7.15-7.26 (m, 3 arom. H), 7.94 (bs, NH). ¹⁹F-NMR (CDCl₃): −220.3 (s). LCMS (M+H): expected for 12b: M=369.49; found: 370.4.

9,10-Didehydro-N,N-diethyl-6-(2-fluoro-1-propen-3-yl)ergoline-8R-carboxamide (TRALA-17), 12c. According to the general procedure for N6 alkylations described, from total 33.9 μL (3×11.3 μL, 2^{nd} addition on day 2, 3^{rd} addition on day 3) 3-bromo-2-fluoro-1-propene and 30 mg 6-Nor-LSD (11), reaction time: 3 days. Yield: 20 mg (56%) TRALA-17 as a yellow foam. Analytical data of 12c as free base: ¹H-NMR (CDCl₃): 1.21 (t, 1×CH₂CH₃), 1.27 (t, 1×CH₂CH₃), 2.75 (t, 1H), 2.99 (t, 1H), 3.22-3.62 (m, 8H), 3.72 (d×d, 1H), 3.85 (bm, 1H), 4.58 (d×d, 1H), 4.77 (d×d, 1H), 6.37 (s, H9), 6.91 (t, 1 arom. H), 7.14-7.26 (m, 3 arom. H), 8.09 (bs, NH). ¹⁹F-NMR (CDCl₃): −97.7 (s). LCMS (M+H): expected for 12c: M=367.47; found: 368.3.

9,10-Didehydro-N,N-diethyl-6-((RS)-(2,2-difluorocyclopropyl)methyl)-ergoline-8R-carboxamide (TRALA-18), 12d. According to the general procedure for N6 alkylation described, from total 60 mg (3×20 mg, 2^{nd} addition on day 2, 3^{rd} addition on day 3) rac. 2-(bromomethyl)-1,1-difluorocyclopropane and 30 mg 6-Nor-LSD (11), reaction time: 3 days. Yield: 11 mg (28%) TRALA-18 as a yellowish solid. Analytical data of 12d as free base: ¹H-NMR (CDCl₃): 1.11 (m, 1H), 1.21 (t, 1×CH₂CH₃), 1.28 (t, 1×CH₂CH₃), 1.53, (m, 1H), 1.84 (m, 1H), 2.62-3.28 (m, 5H), 3.38-3.68 (m, 6H), 3.87 (bm, 1H), 6.37 (s, H9), 6.92 (s, 1 arom. H), 7.14-7.25 (m, 3 arom. H), 8.05 (bs, NH). ¹⁹F-NMR (CDCl₃): −128.7 (m: −128.15; −128.71; −128.75; −129.31), −142.4 (m: −141.63; −142.18; −142.58; −143.14). LCMS (M+H): expected for 12d: M=399.49; found: 400.3.

9,10-Didehydro-N,N-diethyl-6-(cyanomethyl)ergoline-8R-carboxamide (TRALA-19), 12e. According to the general procedure for N6 alkylation described, from total 24.4 μL (2×12.4 μL, 2^{nd} addition on day 2) chloroacetonitrile and 50 mg 6-Nor-LSD (11), reaction time: 2 days. Yield: 33 mg of a yellow foam which still contained some impurities (approx. 15%) based on NMR analytics. Thus, the product was further purified by prep. TLC (DCM/MeOH/NEt_{3=98/2/0.2}) to get 7 mg (13%) TRALA-19 as a yellow foam. Analytical data of 12e as free base: ¹H-NMR (CDCl₃): 1.21 (t, 1×CH₂CH₃), 1.27 (t, 1×CH₂CH₃), 2.71 (t×m, 1H), 3.08 (d×d, 1H), 3.30 (t, 1H), 3.37-3.57 (m, 5H), 3.72 (bm, 1H), 3.76 (d, 1H), 3.91 (bm, 1H), 4.07 (d, 1H), 6.37 (s, H9), 6.94 (t, 1 arom. H), 7.15-7.26 (m, 3 arom. H), 8.06 (bs, NH). LCMS (M+H): expected for 12e: M=348.45; found: 349.2.

9,10-Didehydro-N,N-diethyl-6-(2-oxopropyl)ergoline-8R-carboxamide (TRALA-20), 12f. According to the general procedure for N6 alkylation described, total 27.9 μL (3×9.3 μL, 2^{nd} addition on day 2, 3^{rd} addition on day 3) chloroacetone and 30 mg 6-Nor-LSD (11), reaction time: 2 days. Yield: 18 mg (51%) TRALA-20 as a yellow-beige foam. Analytical data of 12f as free base: ¹H-NMR (CDCl₃): 1.19 (t, 1×CH₂CH₃), 1.27 (t, 1×CH₂CH₃), 2.26 (s, 3H), 2.78 (t, 1H), 2.95 (t, 1H), 3.09 (d×d, 1H), 3.28-3.60 (m, 7H), 3.81-3.95 (m, 2H), 6.39 (s, H9), 6.90 (t, 1 arom. H), 7.13-7.25 (m, 3 arom. H), 8.12 (bs, NH). LCMS (M+H): expected for 12f: M=365.48; found: 366.3.

9,10-Didehydro-N,N-diethyl-6-benzylergoline-8R-carboxamide (TRALA-21), 12g. According to the general procedure for N6 alkylations described, from 13.8 μL benzyl bromide and 30 mg 6-Nor-LSD (11), reaction time: 2.5h. Yield: 16 mg (41%) TRALA-21 as a beige foam. Analytical data of 12g as free base: ¹H-NMR (CDCl₃): 1.14 (m, 2×CH2CH3), 2.83 (m, 2H), 3.10 (d×d, 1H), 3.25-3.52 (m, 5H), 3.57 (m, 1H), 3.73 (m, 2H), 4.36 (d, 1H), 6.38 (s, H9), 6.92 (t, 1 arom. H), 7.13-7.23 (m, 3 arom. H), 7.24-7.38 (m, 3 arom. H), 7.44 (m, 2 arom. H), 8.14 (bs, NH). LCMS (M+H): expected for 12g: M=399.54; found: 400.3.

9,10-Didehydro-N,N-diethyl-6-cyclopropylergoline-8R-carboxamide (TRALA-22), 13. The procedure was adapted from WO2009068214. To a solution of 42.2 mg (136.2 μmol) 6-Nor-LSD (11) in 0.45 mL MeOH anhydr. were added subsequently 41.1 μL (1.5 eq) 1-ethoxy-1-trimethylsiloxycyclopropane, 8.7 μL (1.1 eq) glacial acetic acid and 18 mg (2 eq) NaBH₃CN (caution from HCN vapors when opening the bottle) and the mixture was heated to 60° C. for 4h under nitrogen. The mixture was cooled to ambient temperature, the volatiles were stripped off and the residue was partitioned between ethyl acetate and saturated aq. NaHCO₃. The org. layer was dried over Na₂SO₄ and concentrated in vacuo. The residual crude product was purified with silica gel chromatography using DCM/MeOH/NEt_{3=98/2/0.1} as eluent. Yield: 20 mg (42%) TRALA-22 as a beige foam. Analytical data of 13 as free base: ¹H-NMR (CDCl₃): 0.51 (m, 1H), 0.62 (m, 1H), 0.82 (m, ˜2H), 1.24 (m, 2×CH2CH3), 1.87 (m, 1H), 2.72 (m, 1H), 3.01 (t, 1H), 3.36 (d×d, 1H), 3.42-3.56 (m, 4H), 3.63 (m, 1H), 3.76-3.93 (m, 2H), 6.39 (s, H9), 6.92 (t, 1 arom. H), 7.13-7.25 (m, 3 arom. H), 8.15 (bs, NH). LCMS (M+H): expected for 13: M=349.48; found: 350.3.

9,10-Didehydro-N,N-diethyl-6-cyclobutylergoline-8R-carboxamide (TRALA-23), 14a. To a solution of 42.2 mg (136.2 μmol) 6-Nor-LSD (11) and 15.2 μL (1.5 eq) cyclobutanone in 0.45 mL dichloromethane anhydr. were added 57.6 mg (2 eq) NaBH(OAc)₃ and the mixture was stirred under nitrogen at ambient temperature for 2h. The mixture was diluted with water, stirred for 10 min, diluted with DCM and NaOH 1M was added to a final pH of 8-9. The layers were separated, and the aq. layer was further extracted with 2×DCM. The combined org. layers were dried over Na₂SO₄ and concentrated in vacuo. The residual crude product was purified with silica gel chromatography using DCM/MeOH/NEt₃=98/2/0.1 as eluent. Yield: 13 mg (26%) TRALA-23 as a yellow foam. Analytical data of 14a as free base: ¹H-NMR (CDCl₃): 1.24 (m, 2×CH2CH3), 1.72 (m, 2H), 2.11 (m, 2H), 2.31 (m, 2H), 2.73 (m, 2H), 3.23 (d×d, 1H), 3.36-3.60 (m, 7H), 3.80 (m, 1H), 6.36 (s, H9), 6.90 (t, 1 arom. H), 7.13-7.24 (m, 3 arom. H), 8.06 (bs, NH). LCMS (M+H): expected for 14a: M=363.51; found: 364.4.

9,10-Didehydro-N,N-diethyl-6-(3-oxetanyl)ergoline-8R-carboxamide (TRALA-24), 14b. As described for compound X, from 42.2 mg (136.2 μmol) 6-Nor-LSD (11) and 12 μL (1.5 eq) 3-oxetanone in 0.45 mL dichloromethane anhydr. and 57.6 mg (2 eq) NaBH(OAc)₃. Yield: 23 mg (46%) TRALA-24 as a beige-yellow foam. Analytical data of 14b as free base: ¹H-NMR (CDCl₃): 1.20 (t, CH2CH3), 1.29 (t, CH2CH3), 2.79 (m, 2H), 2.97 (m, 2H), 3.38-3.63 (m, 5H), 3.83 (m, 1H), 4.13 (p, 1H), 4.70 (t, 1H), 4.86 (m, 3H), 6.38 (s, H9), 6.87 (t, 1 arom. H), 7.13-7.23 (m, 3 arom. H), 8.16 (bs, NH). LCMS (M+H): expected for 14b: M=365.48; found: 366.4.

9,10-Didehydro-N,N-diethyl-6-((oxetan-3-yl)methyl)ergoline-8R-carboxamide (TRALA-25), 14c. As described for compound X, from 42.2 mg (136.2 μmol) 6-Nor-LSD (11) and 17.6 mg (1.5 eq) oxetane-3-carbaldehyde in 0.45 mL dichloromethane anhydr. and 57.6 mg (2 eq) NaBH(OAc)₃. Yield: 25 mg (48%) TRALA-25 as a beige foam. Analytical data of 14c as free base: ¹H-NMR (CDCl₃): 1.20 (t, CH2CH3), 1.29 (t, CH2CH3), 2.79 (m, 2H), 2.97 (m, 2H), 3.38-3.63 (m, 5H), 3.83 (m, 1H), 4.13 (p, 1H), 4.70 (t, 1H), 4.86 (m, 3H), 6.38 (s, H9), 6.87 (t, 1 arom. H), 7.13-7.23 (m, 3 arom. H), 8.16 (bs, NH). LCMS (M+H): expected for 14c: M=379.51; found: 380.4.

### Examples—N6-Substituted Derivatives of 6-Nor-TRALA-02, Preparation of Derivatives 16a-c

1.) N6-Demethylation of TRALA-02: Preparation of 6-Nor-TRALA-02.

9,10-Didehydro-N-ethyl-N-propyn-3-ylergoline-8R-carboxamide (6-Nor-TRALA-02), 15. It followed exactly the procedure described for 6-Nor-LSD (11) by using 366 mg (1.1 mmol) TRALA-02 (2b) in 11 mL DCM, 295 mg (1.2 eq) mCPBA and 153 mg (0.5 eq) FeSO₄ heptahydrate in 0.8 mL MeOH. The crude product was purified by silica gel chromatography using DCM/MeOH/NEt₃=95/5/0.1 as eluent. Recovered starting material (2b; 67 mg; 18%) eluted first, followed by the title compound 15, yield: 172 mg (49%) as a tan solid. Analytical data of 15 as free base: ¹H-NMR (CDCl₃): 1.21 (2×t, sum=3H), 2.31 (m, 1H), 2.84 (t×m, 1H), 3.28-3.43 (m, ˜3H), 3.55-3.87 (m, ˜4H), 3.99 (m, 1H), 4.28 (m, 2H), 6.38 (ca. 60%)/6.48 (ca. 40%) (2×s, sum=H9; note; epimeric purity proof of C9 of the ergoline structure: see chapter General; amide couplings), 6.93 (s, 1 arom. H), 7.15-7.27 (m, 3 arom. H), 7.96 (bs, N1H). LCMS (M+H): expected for 15: M=319.41; found: 320.3.

2.) N6-Alkylation of 6-Nor-TRALA-02: Preparation of Compounds 16a-c.

9,10-Didehydro-N-ethyl-N-propyn-3-yl-6-(2-fluoroethyl)ergoline-8R-carboxamide (TRALA-26), 16a. According to the general procedure for N6 alkylations described, from total 28.5 μL (3×9.5 μL, 2^{nd} addition on day 2, 3^{rd} addition after 8h on day 2) 1-fluoro-2-iodoethane and 31 mg 6-Nor-TRALA-02 (15), reaction time: 6 days. Yield: 15 mg (42%) TRALA-26 as a yellow-beige foam. Analytical data of 16a as free base: ¹H-NMR (CDCl₃): 1.32 (m, 3H), 2.31 (m, 1H), 2.73 (t, 1H), 2.99 (m, 2H), 3.28 (m, 2H), 3.45-3.75 (m, 4H), 3.92 (m, 1H), 4.26 (m, 2H), 4.59 (m, 1H), 4.75 (m, 1H), 6.36 (ca. 55%)/6.45 (ca. 45%) (2×s, sum=H9; note; epimeric purity proof of C9 of the ergoline structure: see chapter General; amide couplings), 6.91 (t, 1 arom. H), 7.14-7.26 (m, 3 arom. H), 8.03 (bs, NH). ¹⁹F-NMR (CDCl₃): −218.99, −219.03. LCMS (M+H): expected for 16a: M=365.45; found: 366.2.

9,10-Didehydro-N-ethyl-N-propyn-3-yl-6-(2-fluoro-1-propen-3-yl)ergoline-8R-carboxamide (TRALA-27), 16b. According to the general procedure for N6 alkylations described, from total 33.9 μL (3×11.3 μL, 2^{nd} addition on day 2, 3^{rd} addition after 8h on day 3) 3-bromo-2-fluoro-1-propene and 31 mg 6-Nor-TRALA-02 (15), reaction time: 6 days. Yield: 12 mg (33%) TRALA-27 as a yellow foam. Analytical data of 16b as free base: ¹H-NMR (CDCl₃): 1.31 (m, 3H), 2.31 (m, 1H), 2.74 (t×m, 1H), 3.01 (m, 1H), 3.31 (m, 2H), 3.48-3.78 (m, 5H), 3.89 (m, 1H), 4.27 (m, 2H), 4.59 (d×d, 1H), 4.78 (d×d, 1H), 6.37 (ca. 60%)/6.45 (ca. 40%) (2×s, sum=H9; note; epimeric purity proof of C9 of the ergoline structure: see chapter General; amide couplings), 6.92 (t, 1 arom. H), 7.14-7.26 (m, 3 arom. H), 7.99 (bs, NH). ¹⁹F-NMR (CDCl₃): −97.77, −97.85. LCMS (M+H): expected for 16b: M=377.47; found: 378.3.

9,10-Didehydro-N-ethyl-N-propyn-3-yl-6-((RS)-(2,2-difluorocyclopropyl)-methyl)ergoline-8R-carboxamide (TRALA-28), 16c. According to the general procedure for N6 alkylations described, from total 59.4 μL (3×19.8 μL, 2^{nd} addition on day 2, 3^{rd} addition after 8h on day 3) rac. 2-(bromomethyl)-1,1-difluorocyclopropane and 31 mg 6-Nor-TRALA-02 (15), reaction time: 6 days. Yield: 13 mg (33%) TRALA-28 as a yellow foam. Analytical data of 16c as free base: ¹H-NMR (CDCl₃): 1.11 (m, 1H), 1.31 (m, 3H), 1.52 (m, 1H), 1.84 (m, 1H), 2.31 (m, 1H), 2.61-3.35 (m, 5H), 3.47-3.78 (m, 4H), 3.91 (m, 1H), 4.29 (m, 2H), 6.37 (ca. 50%)/6.45 (ca. 50%) (2×s, sum=H9; note; epimeric purity proof of C9 of the ergoline structure: see chapter General; amide couplings), 6.92 (s, 1 arom. H), 7.14-7.26 (m, 3 arom. H), 8.00 (bs, NH). ¹⁹F-NMR (CDCl₃): −141.65, −141.73, −142.20, −142.28, −142.57, −142.60, −143.13, −143.16. LCMS (M+H): expected for 16c: M=409.48; found: 410.3.

Microsomal assays: The objective of this experiment was the investigation of the microsomal stability of 10 novel lysergamides (FIGS. 13A-13J) in order to make predictions about whether any of the derivatives may be clinically faster metabolized than lysergic acid diethylamide (LSD). The test substances were incubated with human liver microsomes for 4 hours and the metabolic degradation was then measured by liquid chromatography-tandem mass spectrometry (LC-MS/MS). In brief, LSD and the derivatives (10 nM) were incubated in the presence of pooled human liver microsomes for 4 hours. The microsomal reaction mixture contained 464.5 μL phosphate-buffered saline, 25 μL nicotinamide adenine dinucleotide phosphate (NADPH) solution A (1:20 dilution, #451220, lot: 0344003; Corning Life Sciences B.V) and 5 μL Solution B (1:100 dilution, #451200, lot: 0342002; Corning Life Sciences B.V), 5 μL liver microsomes (150 donors, 20 mg/mL, #452117, lot: 38296; Corning Life Sciences B.V., Amsterdam, The Netherlands), and 0.5 μL test drugs (10 μM). Samples (50 μL) were taken 2 minutes prior to microsomal incubation (t0) and 0.5, 1, 2, 3, and 4 hours after initiation of the microsomal reaction. A total of five assays were performed per substance. The amount of the test substances (peak area) exposed to human liver microsomes for 4 hours was compared relative to a concentration of 10 nM (t0, 100% peak area). Linear regression slopes were plotted to compare the degradation of the derivatives and LSD. None of the TRALA derivatives were metabolized to LSD (data not shown).

Throughout this application, various publications, including United States patents, are referenced by author and year and patents by number. Full citations for the publications are listed herein. The disclosures of these publications and patents in their entireties are hereby incorporated by reference into this application in order to more fully describe the state of the art to which this invention pertains.

The invention has been described in an illustrative manner and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Abramson, H. A. (1959). Lysergic acid diethylamide (LSD-25): XXIX. The Response Index as a Measure of Threshold Activity of Psychotropic Drugs in Man. J Psychol, 48(1), 65-78.
2. Bailey, K., Verner, D., & Legault, D. (1973). Distinction of Some Dialkyl Amides of Lysergic and iso-Lysergic Acids from LSD. J Assoc Off Anal Chem, 56, 88-99.
3. Becker, A. M., Holze, F., Grandinetti, T., Klaiber, A., Toedtli, V. E., Kolaczynska, K. E., Liechti, M. E. (2022). Acute effects of psilocybin after escitalopram or placebo pretreatment in a randomized, double-blind, placebo-controlled, crossover study in healthy subjects. Clin Pharmacol Ther, 111, 886-895. doi:10.1002/cpt.2487
4. Bogenschutz, M. P. (2013). Studying the effects of classic hallucinogens in the treatment of alcoholism: rationale, methodology, and current research with psilocybin. Curr Drug Abuse Rev, 6(1), 17-29. Retrieved from http://www.ncbi.nlm.nih.go/pubmed/23627783
5. Bogenschutz, M. P., Forcehimes, A. A., Pommy, J. A., Wilcox, C. E., Barbosa, P. C., & Strassman, R. J. (2015). Psilocybin-assisted treatment for alcohol dependence: a proof-of-concept study. J Psychopharmacol, 29(3), 289-299. doi:10.1177/0269881114565144
6. Brandt, S. D., Kavanagh, P. V., Westphal, F., Elliott, S. P., Wallach, J., Colestock, T., Halberstadt, A. L. (2017). Return of the lysergamides. Part II: Analytical and behavioural characterization of N(6)-allyl-6-norlysergic acid diethylamide (AL-LAD) and (2′S,4′S)-lysergic acid 2,4-dimethylazetidide (LSZ). Drug Test Anal, 9(1), 38-50. doi:10.1002/dta.1985
7. Brandt, S. D., Kavanagh, P. V., Westphal, F., Stratford, A., Odland, A. U., Klein, A. K., Halberstadt, A. L. (2020). Return of the lysergamides. Part VI: Analytical and behavioural characterization of 1-cyclopropanoyl-d-lysergic acid diethylamide (1CP-LSD). Drug Test Anal, 12(6), 812-826. doi:10.1002/dta.2789
8. Canezin, J., Cailleux, A., Turcant, A., Le Bouil, A., Harry, P., & Allain, P. (2001). Determination of LSD and its metabolites in human biological fluids by high-performance liquid chromatography with electrospray tandem mass spectrometry. J Chromatogr B Biomed Sci Appl, 765(1), 15-27. Retrieved from http://www.ncbi.nlm.nih.gov/pubmed/11817305
9. Cao, D., Yu, J., Wang, H., Luo, Z., Liu, X., He, L., Wang, S. (2022). Structure-based discovery of nonhallucinogenic psychedelic analogs. Science, 375(6579), 403-411. doi:10.1126/science.abl8615
10. Carhart-Harris, R., Giribaldi, B., Watts, R., Baker-Jones, M., Murphy-Beiner, A., Murphy, R., . . . Nutt, D. J. (2021). Trial of psilocybin versus escitalopram for depression. N Engl J Med, 384(15), 1402-1411. doi:10.1056/NEJMoa2032994
11. Carhart-Harris, R. L., Bolstridge, M., Rucker, J., Day, C. M., Erritzoe, D., Kaelen, M., Nutt, D. J. (2016). Psilocybin with psychological support for treatment-resistant depression: an open-label feasibility study. Lancet Psychiatry, 3, 619-627. doi:10.1016/S2215-0366(16)30065-7
12. Carhart-Harris, R. L., Kaelen, M., Bolstridge, M., Williams, T. M., Williams, L. T., Underwood, R., Nutt, D. J. (2016). The paradoxical psychological effects of lysergic acid diethylamide (LSD). Psychol Med, 46, 1379-1390. doi:10.1017/S0033291715002901
13. Davis, A. K., Barrett, F. S., May, D. G., Cosimano, M. P., Sepeda, N. D., Johnson, M. W., Griffiths, R. R. (2021). Effects of psilocybin-assisted therapy on major depressive disorder: a randomized clinical trial. JAMA Psychiatry, 78(5), 481-489. doi:10.1001/jamapsychiatry.2020.3285
14. Dolder, P. C., Schmid, Y., Mueller, F., Borgwardt, S., & Liechti, M. E. (2016). LSD acutely impairs fear recognition and enhances emotional empathy and sociality. Neuropsychopharmacology, 41, 2638-2646.
15. Dolder, P. C., Schmid, Y., Steuer, A. E., Kraemer, T., Rentsch, K. M., Hammann, F., & Liechti, M. E. (2017). Pharmacokinetics and pharmacodynamics of lysergic acid diethylamide in healthy subjects. Clin Pharmacokinetics, 56, 1219-1230.
16. Dominguez-Clave, E., Soler, J., Elices, M., Pascual, J. C., Alvarez, E., de la Fuente Revenga, M., Riba, J. (2016). Ayahuasca: Pharmacology, neuroscience and therapeutic potential. Brain Res Bull, 126 (Pt 1), 89-101. doi:10.1016/j.brainresbull.2016.03.002
17. Dos Santos, R. G., Osorio, F. L., Crippa, J. A., Riba, J., Zuardi, A. W., & Hallak, J. E. (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol, 6(3), 193-213. doi:10.1177/2045125316638008
18. Fehr, T., Stadler, P. A., & Hofmann, A. (1970). [Demethylation of lysergic acid skeleton]. Helv Chim Acta, 53(8), 2197-2201. doi:10.1002/hlca.19700530832
19. Garcia-Romeu, A., Davis, A. K., Erowid, F., Erowid, E., Griffiths, R. R., & Johnson, M. W. (2019). Cessation and reduction in alcohol consumption and misuse after psychedelic use. J Psychopharmacol, 33(9), 1088-1101. doi:10.1177/0269881119845793
20. Garcia-Romeu, A., Griffiths, R. R., & Johnson, M. W. (2014). Psilocybin-occasioned mystical experiences in the treatment of tobacco addiction. Curr Drug Abuse Rev, 7(3), 157-164. Retrieved from http//www.ncbi.nih.gov/pubmed/25563
21. Gasser, P., Holstein, D., Michel, Y., Doblin, R., Yazar-Klosinski, B., Passie, T., & Brenneisen, R. (2014). Safety and efficacy of lysergic acid diethylamide-assisted psychotherapy for anxiety associated with life-threatening diseases. J Nerv Ment Dis, 202(7), 513-520. doi:10.1097/NMD.0000000000000113
22. Gasser, P., Kirchner, K., & Passie, T. (2015). LSD-assisted psychotherapy for anxiety associated with a life-threatening disease: a qualitative study of acute and sustained subjective effects. J Psychopharmacol, 29(1), 57-68. doi:10.1177/0269881114555249
23. Golding, B. T., & Wong, A. K. (1981). Preparation of labeled aldehydes and ketones from enamides. Angewandte Chemie, 20, 89-90.
24. Griffiths, R., Richards, W., Johnson, M., McCann, U., & Jesse, R. (2008). Mystical-type experiences occasioned by psilocybin mediate the attribution of personal meaning and spiritual significance 14 months later. J Psychopharmacol, 22(6), 621-632. doi:10.1177/0269881108094300
25. Griffiths, R. R., Johnson, M. W., Carducci, M. A., Umbricht, A., Richards, W. A., Richards, B. D., Klinedinst, M. A. (2016). Psilocybin produces substantial and sustained decreases in depression and anxiety in patients with life-threatening cancer: a randomized double-blind trial. J Psychopharmacol, 30(12), 1181-1197. doi:10.1177/0269881116675513
26. Grob, C. S., Danforth, A. L., Chopra, G. S., Hagerty, M., McKay, C. R., Halberstadt, A. L., & Greer, G. R. (2011). Pilot study of psilocybin treatment for anxiety in patients with advanced-stage cancer. Arch Gen Psychiatry, 68(1), 71-78. doi:10.1001/archgenpsychiatry.2010.116
27. Halberstadt, A. L., Chatha, M., Klein, A. K., McCorvy, J. D., Meyer, M. R., Wagmann, L., Brandt, S. D. (2020). Pharmacological and biotransformation studies of 1-acyl-substituted derivatives of d-lysergic acid diethylamide (LSD). Neuropharmacology, 172, 107856. doi:10.1016/j.neuropharm.2019.107856
28. He, L., Zhao, L., Wang, D. X., & Wang, M. X. (2014). Catalytic asymmetric difunctionalization of stable tertiary enamides with salicylaldehydes: highly efficient, enantioselective, and diastereoselective synthesis of diverse 4-chromanol derivatives. Org Lett, 16(22), 5972-5975. doi:10.1021/ol5029964
29. Hoehn, R. D., Nichols, D. E., McCorvy, J. D., Neven, H., & Kais, S. (2017). Experimental evaluation of the generalized vibrational theory of G protein-coupled receptor activation. Proc Natl Acad Sci USA, 114(22), 5595-5600. doi:10.1073/pnas.1618422114
30. Hoffman, A. J., & Nichols, D. E. (1985). Synthesis and LSD-like discriminative stimulus properties in a series of N(6)-alkyl norlysergic acid N,N-diethylamide derivatives. J Med Chem, 28(9), 1252-1255. doi:10.1021/jm00147a022
31. Hoffmann, A. J. (1987). Synthesis and pharmacological evaluation of N(6)-alkyl norlyergic acid N,N-diethylamide derivatives. In Department of Pharmacology: Perdue University.
32. Holze, F., Caluori, T. V., Vizeli, P., & Liechti, M. E. (2021). Safety pharmacology of acute LSD administration in healthy subjects. Psychopharmacology (Berl) doi:10.1007/s00213-021-05978-6
33. Holze, F., Duthaler, U., Vizeli, P., Muller, F., Borgwardt, S., & Liechti, M. E. (2019). Pharmacokinetics and subjective effects of a novel oral LSD formulation in healthy subjects. Br J Clin Pharmacol, 85, 1474-1483. doi:10.1111/bcp.13918
34. Holze, F., Ley, L., Muller, F., Becker, A. M., Straumann, I., Vizeli, P., Liechti, M. E. (2022). Direct comparison of the acute effects of lysergic acid diethylamide and psilocybin in a double-blind placebo-controlled study in healthy subjects. Neuropsychopharmacology, doi.org/10.1038/s41386-022-01297-2.
35. Holze, F., Vizeli, P., Ley, L., Muller, F., Dolder, P., Stocker, M., Liechti, M. E. (2021). Acute dose-dependent effects of lysergic acid diethylamide in a double-blind placebo-controlled study in healthy subjects. Neuropsychopharmacology, 46(3), 537-544. doi:10.1038/s41386-020-00883-6
36. Holze, F., Vizeli, P., Muller, F., Ley, L., Duerig, R., Varghese, N., Liechti, M. E. (2020). Distinct acute effects of LSD, MDMA, and D-amphetamine in healthy subjects. Neuropsychopharmacology, 45(3), 462-471. doi:10.1038/s41386-019-0569-3
37. Hu, Y., Chan, K. H., He, X., Ho, M. K., & Wong, Y. H. (2014). Synthesis and functional characterization of substituted isoquinolinones as MT2-selective melatoninergic ligands. PLoS One, 9(12), el 13638. doi:10.1371/journal.pone.0113638
38. Huang, X., Marona-Lewicka, D., Pfaff, R. C., & Nichols, D. E. (1994). Drug discrimination and receptor binding studies of N-isopropyl lysergamide derivatives. Pharmacol Biochem Behav, 47(3), 667-673. doi:10.1016/0091-3057(94)90172-4
39. Hysek, C. M., Simmler, L. D., Nicola, V., Vischer, N., Donzelli, M., KrAhenbuhl, S., Liechti, M. E. (2012). Duloxetine inhibits effects of MDMA (“ecstasy”) in vitro and in humans in a randomized placebo-controlled laboratory study. PLoS One, 7, e36476.
40. Ichikawa, J., & Meltzer, H. Y. (2000). The effect of serotonin(1A) receptor agonism on antipsychotic drug-induced dopamine release in rat striatum and nucleus accumbens. Brain Res, 858(2), 252-263. doi:10.1016/s0006-8993(99)02346-x
41. Johnson, M. W., Garcia-Romeu, A., Cosimano, M. P., & Griffiths, R. R. (2014). Pilot study of the 5-HT2AR agonist psilocybin in the treatment of tobacco addiction. J Psychopharmacol, 28(11), 983-992. doi:10.1177/0269881114548296
42. Johnson, M. W., Garcia-Romeu, A., & Griffiths, R. R. (2016). Long-term follow-up of psilocybin-facilitated smoking cessation. Am J Drug Alcohol Abuse, 43, 55-60. doi:10.3109/00952990.2016.1170135
43. Krebs, T. S., & Johansen, P. O. (2012). Lysergic acid diethylamide (LSD) for alcoholism: meta-analysis of randomized controlled trials. J Psychopharmacol, 26(7), 994-1002. doi:10.1177/0269881112439253
44. Kulyashova, A., & M., K. (2016). Convenient modular construction of medicinally important5-acylamino-4,5-dihydroisoxazoles featuring four elements of diversity. Tetrahedron Lett, 57, 4395-4397.
45. Liechti, M. E. (2017). Modern clinical research on LSD. Neuropsychopharmacology, 42, 2114-2127.
46. Luethi, D., Hoener, M. C., Krahenbuhl, S., Liechti, M. E., & Duthaler, U. (2019). Cytochrome P450 enzymes contribute to the metabolism of LSD to nor-LSD and 2-oxo-3-hydroxy-LSD: Implications for clinical LSD use. Biochem Pharmacol, 164, 129-138. doi:10.1016/j.bcp.2019.04.013
47. Luethi, D., & Liechti, M. E. (2018). Monoamine transporter and receptor interaction profiles in vitro predict reported human doses of novel psychoactive stimulants and psychedelics. Int J Neuropsychopharmacol, 21(10), 926-931. doi:10.1093/ijnp/pyy047
48. Madsen, M. K., Fisher, P. M., Burmester, D., Dyssegaard, A., Stenbaek, D. S., Kristiansen, S., Knudsen, G. M. (2019). Correction: Psychedelic effects of psilocybin correlate with serotonin 2A receptor occupancy and plasma psilocin levels. Neuropsychopharmacology, 44(7), 1328-1334. doi:10.1038/s41386-019-0360-5
49. Marona-Lewicka, D., Thisted, R. A., & Nichols, D. E. (2005). Distinct temporal phases in the behavioral pharmacology of LSD: dopamine D2 receptor-mediated effects in the rat and implications for psychosis. Psychopharmacology (Berl), 180(3), 427-435. doi:10.1007/s00213-005-2183-9
50. McCamley, K., Ripper, J. A., Singer, R. D., & Scammells, P. J. (2003). Efficient N-demethylation of opiate alkaloids using a modified nonclassical Polonovski reaction. J Org Chem, 68(25), 9847-9850. doi:10.1021/jo035243z
51. Meiresonne, T., Verniest, G., De Kimpe, N., & Mangelinckx, S. (2015). Synthesis of 2-Fluoro-1,4-benzoxazines and 2-Fluoro-1,4-benzoxazepin-5-ones by Exploring the Nucleophilic Vinylic Substitution (S(N)V) Reaction of gem-Difluoroenamides. J Org Chem, 80(10), 5111-5124. doi:10.1021/acs.joc.5b00507
52. Meuzelaar, G. J., van Vliet, C. A., Neeleman, E., Maat, L., & Sheldon, R. A. (1997). Synthesis of gamma-unsaturated enamides by N-acylation of imines derived from gamma-unsaturated amines. Liebigs Ann Recueil, 1159-1163.
53. Mimura, H., Kawada, K., Yamshita, T., Sakamato, T., & Kikugawa, Y. (2010). Trifluoroacetaldehyde: A useful industrial bulk material for the synthesis of trifluoromethylated amino compounds. J Fluor Chem, 131, 477-486.
54. Nichols, D. E. (2016). Psychedelics. Pharmacol Rev, 68(2), 264-355. doi:10.1124/pr.115.011478
55. Nichols, D. E. (2018a). Chemistry and Structure-Activity Relationships of Psychedelics. Curr Top Behav Neurosci, 36, 1-43. doi:10.1007/7854_2017_475
56. Nichols, D. E. (2018b). Dark classics in chemical neuroscience: lysergic acid diethylamide (LSD). ACS Chem Neurosci, 9, 2331-2343. doi:10.1021/acschemneuro.8b00043
57. Nichols, D. E., Frescas, S., Marona-Lewicka, D., & Kurrasch-Orbaugh, D. M. (2002). Lysergamides of isomeric 2,4-dimethylazetidines map the binding orientation of the diethylamide moiety in the potent hallucinogenic agent N,N-diethyllysergamide (LSD). J Med Chem, 45(19), 4344-4349. Retrieved from http://www.ncbi.nlm.nih.gov/pubmed/12213075
58. Nichols, D. E., & Grob, C. S. (2018). Is LSD toxic? Forensic Sci Int, 284, 141-145. doi:10.1016/j.forsciint.2018.01.006
59. Nichols, D. E., Monte, A., Huang, X., & Marona-Lewicka, D. (1996). Stereoselective pharmacological effects of lysergic acid amides possessing chirality in the amide substituent. Behav Brain Res, 73(1-2), 117-119. doi:10.1016/0166-4328(96)00080-0
60. Oberlender, R., Pfaff, R. C., Johnson, M. P., Huang, X. M., & Nichols, D. E. (1992). Stereoselective LSD-like activity in d-lysergic acid amides of (R)- and (S)-2-aminobutane. J Med Chem, 35(2), 203-211. doi:10.1021/jm00080a001
61. Palhano-Fontes, F., Barreto, D., Onias, H., Andrade, K. C., Novaes, M. M., Pessoa, J. A., Araujo, D. B. (2019). Rapid antidepressant effects of the psychedelic ayahuasca in treatment-resistant depression: a randomized placebo-controlled trial. Psychol Med, 49(4), 655-663. doi:10.1017/S0033291718001356
62. Passie, T., Halpern, J. H., Stichtenoth, D. O., Emrich, H. M., & Hintzen, A. (2008). The pharmacology of lysergic acid diethylamide: a review. CNS Neurosci Ther, 14(4), 295-314. doi:10.1111/j.1755-5949.2008.00059.x
63. Pfaff, R. C., Huang, X., Marona-Lewicka, D., Oberlender, R., & Nichols, D. E. (1994). Lysergamides revisited. NIDA Res Monogr, 146, 52-73. Retrieved from https://www.ncbi.nlm.nih.gov/pubmed/87/42794
64. Preller, K. H., Herdener, M., Pokorny, T., Planzer, A., Kraehenmann, R., StAmpfli, P., Vollenweider, F. X. (2017). The fabric of meaning and subjective effects in LSD-induced states depend on serotonin 2A receptor activation Curr Biol, 27, 451-457.
65. Rickli, A., Moning, O. D., Hoener, M. C., & Liechti, M. E. (2016). Receptor interaction profiles of novel psychoactive tryptamines compared with classic hallucinogens. Eur Neuropsychopharmacol, 26, 1327-1337.
66. Riss, P. J., & Aigbirhio, F. I. (2011). A simple, rapid procedure for nucleophilic radiosynthesis of aliphatic [18F]trifluoromethyl groups. Chem Commun (Camb), 47(43), 11873-11875. doi:10.1039/c1cc15342k
67. Roseman, L., Nutt, D. J., & Carhart-Harris, R. L. (2017). Quality of acute psychedelic experience predicts therapeutic efficacy of psilocybin for treatment-resistant depression. Front Pharmacol, 8, 974. doi:10.3389/fphar.2017.00974
68. Ross, S., Bossis, A., Guss, J., Agin-Liebes, G., Malone, T., Cohen, B., Schmidt, B. L. (2016). Rapid and sustained symptom reduction following psilocybin treatment for anxiety and depression in patients with life-threatening cancer: a randomized controlled trial. J Psychopharmacol, 30(12), 1165-1180. doi:10.1177/0269881116675512
69. Sanches, R. F., de Lima Osorio, F., Dos Santos, R. G., Macedo, L. R., Maia-de-Oliveira, J. P., Wichert-Ana, L., Hallak, J. E. (2016). Antidepressant Effects of a Single Dose of Ayahuasca in Patients With Recurrent Depression: A SPECT Study. J Clin Psychopharmacol, 36(1), 77-81. doi:10.1097/jcp.0000000000000436
70. Schmid, Y., Enzler, F., Gasser, P., Grouzmann, E., Preller, K. H., Vollenweider, F. X., Liechti, M. E. (2015). Acute effects of lysergic acid diethylamide in healthy subjects. Biol Psychiatry, 78(8), 544-553. doi: 10.1016/j.biopsych.2014.11.015
71. Schmid, Y., Gasser, P., Oehen, P., & Liechti, M. E. (2021). Acute subjective effects in LSD- and MDMA-assisted psychotherapy. J Psychopharmacol, 35(4), 362-374. doi:10.1177/0269881120959604
72. Schmid, Y., & Liechti, M. E. (2018). Long-lasting subjective effects of LSD in normal subjects. Psychopharmacology (Ber/), 235(2), 535-545. doi:10.1007/s00213-017-4733-3
73. Shulgin, A., & Shulgin, A. (1991). PiHKAL: a chemical love story (1 edn).
74. Shulgin, A. T., & Shulgin, A. (1997). TiHKAL the continuation: Berkeley: Transform Press.
75. Spiess, P., Berger, M., Kaiser, D., & Maulide, N. (2021). Direct Synthesis of Enamides via Electrophilic Activation of Amides. J Am Chem Soc, 143(28), 10524-10529. doi:10.1021/jacs.1c04363
76. Stachulski, A. V., Nichols, D. E., & Scheinmann, F. (1996). Stereochemical and NMR Reassignment of 6-Norlysergic Acid Diethylamide and 6-Nor-6-allyllysergic Acid Diethylamide. J Chem Res, S1, 30-31.
77. Studerus, E., Gamma, A., Kometer, M., & Vollenweider, F. X. (2012). Prediction of psilocybin response in healthy volunteers. PLoS One, 7(2), e30800. doi:10.1371/journal.pone.0030800
78. Taniguchi, T., Ishita, A., Uchiyama, M., Tamura, O., Muraoka, O., Tanabe, G., & Ishibashi, H. (2005). 7-endo selective aryl radical cyclization onto enamides leading to 3-benzazepines: concise construction of a cephalotaxine skeleton. J Org Chem, 70(5), 1922-1925. doi:10.1021/jo040264u
79. Vizeli, P., Straumann, I., Holze, F., Schmid, Y., Dolder, P. C., & Liechti, M. E. (2021). Genetic influence of CYP2D6 on pharmacokinetics and acute subjective effects of LSD in a pooled analysis. Sci Rep, 11(1), 10851. doi:10.1038/s41598-021-90343-y
80. Vollenweider, F. X., Vontobel, P., Hell, D., & Leenders, K. L. (1999). 5-HT modulation of dopamine release in basal ganglia in psilocybin-induced psychosis in man—a PET study with [11C]raclopride. Neuropsychopharmacology, 20(5), 424-433. doi:10.1016/S0893-133X(98)00108-0
81. Wacker, D., Wang, S., McCorvy, J. D., Betz, R. M., Venkatakrishnan, A. J., Levit, A., Roth, B. L. (2017). Crystal structure of an LSD-bound human serotonin receptor. Cell, 168(3), 377-389.e312. doi:10.1016/j.cell.2016.12.033
82. Watts, V. J., Lawler, C. P., Fox, D. R., Neve, K. A., Nichols, D. E., & Mailman, R. B. (1995). LSD and structural analogs: pharmacological evaluation at D1 dopamine receptors. Psychopharmacology (Berl), 118(4), 401-409. Retrieved from http://www.ncbi.nhn.nih.gov/pubmed/7568626
83. Xu, Y., Xiao-Yu, L., Wang, Z., & Tang, L. (2017). A convenient synthesis of N-vinyl enamides via the lithiation and ring-opening reaction of 2-phenyl-2-oxazolines Tetrahedron Lett, 58, 1788-1791.

1. A pharmacologically active compound
characterized in that it exhibits a structural composition as represented by FIG. 1A,
further characterized in that it is part of class 1 to class 5;
and such compounds are represented as shown in FIG. 2A to FIG. 6C wherein:
class 1 is a lysergic acid amide as represented in FIGS. 2A-2G and FIGS. 3A-3H, wherein R8′ is consisting of substituents shown in subclasses, named subclasses 1a to 1n, whereby R8 is consisting of
a) R8′,
b) any substituent of the subclasses 1a to 1n and R8′═ as defined in the specific class from 1a to 1l,
c) Hydrogen, C₁-C₅ alkyl, branched C₁-C₅ alkyl, C₃-C₅ cycloalkyl, C₁-C₅ alkylcycloalkyl, C₂-C₅ alkenyl, branched C₃-C₅ alkenyl, C₂-C₅ alkynyl, branched C₄-C₅ alkynyl, or
d) as specifically indicated in subclasses 1a to 1n;
with that defined,
in subclass 1a, the substituent R8′ consists of an F₁-F₁₁ fluorine substituted C1-C5 alkyl or branched C₃-C₅ alkyl group, each optionally combined with D₁-D₁₀ deuteron, and/or hydroxy and/or carbonyl,
in subclass 1b, the substituent R8′ consists of an F1-F13 fluorine substituted C3-C7 alkenyl group, optionally combined with D1-D12 deuteron, and/or nitrile, and/or hydroxy and/or carbonyl, whereby the double bond being isolated from the Nitrogen,
in subclass 1c, the substituent R8′ consists of an F1-F11 fluorine substituted C3-C6 cycloalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in subclass 1d, the substituent R8′ consists of an F1-F17 fluorine substituted C3-C6 cycloalkylalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in subclass 1e, the substituent R8′ consists of an F1-F11 fluorine substituted C3-C7 alkynyl group, optionally combined with D1-D12 deuteron, and/or nitrile, and/or hydroxy and/or carbonyl, with the triple bond isolated from the amide Nitrogen,
in subclass 1f, the substituent R8′ consists of an F0-F7 fluorine substituted C2-C4 alkenyl group attached to the Nitrogen with the unsaturated part, yielding enamides, optionally combined with D1-D7 deuteron, and/or nitrile, and/or hydroxy and/or carbonyl,
in subclass 1g, the substituent R8′ consists of an F1-F5 fluorine substituted C2-C4 alkylalkynyl group attached to the Nitrogen with the unsaturated part, yielding ynamides, optionally combined with D1-D4 deuteron, and/or nitrile, and/or hydroxy and/or carbonyl,
in class 1 h, being a subclass of class 1, the substituent R8′ consists of an F1-F13 fluorine substituted C1-3-O—C1-3 alkoxyalkyl group, optionally combined with D1-D12 deuteron, and/or nitrile, and/or hydroxy and/or carbonyl,
in subclass 1i, the substituent R8′ consists of an F0-F7 fluorine substituted C1-C3 alkoxy or C3-C4 cycloalkoxy group, each optionally combined with D1-D7 deuteron, and/or nitrile, and/or hydroxy and/or carbonyl,
in subclass 1j, the substituent R8′ consists of a nitrile attached to a C1-C3 alkyl group, optionally combined with F1-F7 fluorine, and/or D1-D7 deuteron, and/or hydroxy and/or carbonyl,
in subclass 1k, the substituent R8 consists of any D1-D6 deuteron combined with F1-F6 fluorine containing C1-C3 alkyl group optionally combined with hydroxy and/or carbonyl, and R8′ consists of a Hydrogen, a C1-C6 alkyl or a C3-C5 cycloalkyl or a C4-C7 cycloalkylalkyl group, optionally combined with hydroxy and/or carbonyl,
in subclass 1l, the substituent R8 consists of a D1-D7 deuteron or an F1-F7 fluorine, or of any D1-D6 deuteron combined with F1-F6 fluorine containing C1-C3 alkyl group optionally combined with hydroxy and/or carbonyl, and R8′ consists of a C2-C8 alkenyl or a C2-C8 alkynyl group, optionally combined with nitrile, and/or hydroxy and/or carbonyl,
in subclass 1m, the substituent R8 and R8′ are connected to each other to build an azacycloalkane with the amide Nitrogen, and are consisting of a D1-D10 deuteron or an F1-F10 fluorine, or of any D1-D9 deuteron combined with F1-F9 fluorine containing C3-C6 alkylene group optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group, and
in subclass 1n, the substituent R8 and R8′ are connected to each other to build an azacycloalkane with the amide Nitrogen and are consisting of a C3-C6 alkylene group having a nondeuterated or deuterated C1-C3 alkenyl and/or a nondeuterated or deuterated C2-C3 alkynyl group attached, the azacycloalkane forming alkylene group further and optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group;
class 2 is a lysergic acid amide as represented in FIGS. 4A-4G and FIGS. 5A-5C, further characterized in that it consists of 6-substituted 6-Nor-lysergic acid diethylamides, wherein R6 is consisting of substituents shown in subclasses, named subclasses 2a to 2i, whereby R6 is characterized as follows:
in subclass 2a, the substituent R6 consists of an F₁-F₁₁ fluorine substituted C1-C5 alkyl or branched C₃-C₅ alkyl group, each optionally combined with D₁-D₁₀ deuteron, and/or nitrile, and/or hydroxy and/or carbonyl,
in subclass 2b, being a subclass of class 2, the substituent R6 consists of an F1-F13 fluorine substituted C3-C7 alkenyl group, optionally combined with D1-D12 deuteron, and/or nitrile, and/or hydroxy and/or carbonyl, with the alkenyl double bond being isolated from Nitrogen,
in subclass 2c, the substituent R6 consists of an F1-F11 fluorine substituted C3-C7 alkynyl group with the triple bond isolated from N6 Nitrogen, optionally combined with D1-D10 deuteron, and/or nitrile, and/or hydroxy and/or carbonyl. In case the substituent R6 contains at least one nitrile, one hydroxy or one carbonyl group, R6 can also consist of a C3-C7 alkynyl group with the triple bond isolated from N6 Nitrogen, optionally combined with D1-D10 deuteron, and/or nitrile, and/or hydroxy and/or carbonyl,
in subclass 2d, the substituent R6 consists of a C3-C6 cycloalkyl group, optionally combined with F1-F11 fluorine, and/or D1-D11 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl, in subclass 2e, the substituent R6 consists of an F1-F17 fluorine substituted C4-C9 cycloalkylalkyl group, optionally combined with D1-D16 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl. In case the substituent R6 is not cyclopropylmethyl attached by the exocyclic methylene unit to the N6 Nitrogen of the ergoline structure, or it is cyclopropylmethyl attached by the exocyclic methylene unit to the N6 Nitrogen of the ergoline structure and contains at least one nitrile, one hydroxy or one carbonyl group, R6 can also consist of a C4-C9 cycloalkylalkyl group, optionally combined with D1-D17 deuteron, and/or nitrile, and/or hydroxy, and/or carbonyl, and/or deuterated and nondeuterated C1-C3 alkyl and/or deuterated and nondeuterated C1-C3 alkenyl and/or deuterated and nondeuterated C1-C3 alkynyl group,
in subclass 2f, the substituent R6 consists of an F0-F7 fluorine substituted C2-C4 alkenyl group attached to the Nitrogen with the unsaturated part, yielding enamines, optionally combined with D1-D7 deuteron, and/or nitrile, and/or hydroxy and/or carbonyl,
in subclass 2g, the substituent R6 consists of a C3-C6 oxacycloalkyl, a C3-C9 oxacycloalkylalkyl, a C3-C6 thiacycloalkyl or of a C3-C9 thiacycloalkylalkyl group, each optionally combined with F1-F19 fluorine, and/or D1-D19 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in subclass 2h, the substituent R6 consists of an F0-F11 fluorine substituted C1-C5 alkoxy or C3-C6 cycloalkoxy or C2-C6 alkenoxy group, each optionally combined with D1-D11 deuteron, and/or nitrile, and/or hydroxy and/or carbonyl,
in subclass 2i, the substituent R6 consists of an aryl, a heteroaryl, an arylmethyl or a heteroarylmethyl group, each optionally combined with F0-F7 fluorine, and/or D1-D7 deuteron, and/or one or more of the following substituents that themselves can optionally be fluorinated and/or deuterated: halogen, nitrile, nitro, hydroxy, carbonyl, C1-C4 alkoxy, C1-C4 alkyl, C1-C4 alkenyl, C1-C4 alkynyl, C3-C6 cycloalkyl, C3-C6 cycloalkoxy, C3-C6 alkenoxy, C3-C6 alkynoxy, methylenedioxy, C1-C4 alkylthio, C3-C6 alkenylthio, C3-C6 alkynylthio, C3-C6 cycloalkylthio, and the R6 substituent, as defined for class 2i above, can further be annulated;
class 3 is a lysergic acid amide as represented in FIG. 6A, further characterized in that it consists of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C);
class 4 is a lysergic acid amide as represented in FIG. 6B, further characterized in that it consists of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group:
a) any acyl;
b) unsubstituted and substituted carbamoyl;
c) amide-bound amino acid;
d) alkyl, alkenyl or alkynyl;
e) alkoxy, alkenoxy or alkynoxy;
f) any of the substituents described under a) to e), substituted with one or more fluorine atoms;
g) any of the substituents described under a) to e), substituted with one or more deuteron atoms; h) any of the substituents described under a) to e), substituted with one or more fluorine atoms and one or more deuteron atoms; and
class 5 (FIG. 6C), consisting of a monodeuterated up to a fully deuterated ergoline core structure, and additionally consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) Hydrogen; b) any acyl; c) unsubstituted and substituted carbamoyl; d) amide-bound amino acid; e) alkyl, alkenyl or alkynyl; f) alkoxy, alkenoxy or alkynoxy; g) any of the substituents described under a) to f), substituted with one or more fluorine atoms; h) any of the substituents described under a) to f), substituted with one or more deuteron atoms; i) any of the substituents described under a) to f), substituted with one or more fluorine atoms and one or more deuteron atoms.

2. A pharmacologically active compound of claim 1, further characterized in that the compound is a free base.

3. A pharmacologically active compound of claim 1, further characterized in that the compound is a salt thereof.

4. A pharmacologically active compound of claim 3, further characterized in that the compound is a tartrate salt or a hemitartrate salt thereof.

5. A pharmacologically active compound of claim 4, further characterized in that the compound is a pharmacologically acceptable acid addition salt thereof.

6. A pharmacologically active compound of claim 1, further characterized in that the compound is chosen from the group consisting of a racemate, a single enantiomer, a diastereomer, an epimer, and a mixture of enantiomers or diastereomers or epimers in any ratio, a single and a mixture E or Z configurational isomer in any ratio, a single and a mixture cis or trans configurational isomer in any ratio and any combination thereof.

7. A method of changing neurotransmission, including the steps of:
administering a pharmaceutically effective amount of composition to a mammal of a pharmacologically active compound
characterized in that this compound exhibits a structural composition as represented by FIG. 1A,
further characterized in that it is part of class 1 to class 5;
and such compounds are represented as shown in FIG. 2A to FIG. 6C, wherein:
class 1 is a lysergic acid amide as represented in FIGS. 2A-2G and FIGS. 3A-3H, wherein R8′ is consisting of substituents shown in subclasses, named subclasses 1a to 1n, whereby R8 is consisting of
a) R8′,
b) any substituent of the subclasses 1a to 1n and R8′═ as defined in the specific class from 1a to 11,
c) Hydrogen, C₁-C₅ alkyl, branched C₁-C₅ alkyl, C₃-C₅ cycloalkyl, C₁-C₅ alkylcycloalkyl, C₂-C₅ alkenyl, branched C₃-C₅ alkenyl, C₂-C₅ alkynyl, branched C₄-C₅ alkynyl, or
d) as specifically indicated in subclasses 1a to 1n;
with that defined,
in subclass 1a, the substituent R8′ consists of an F₁-F₁₁ fluorine substituted C1-C5 alkyl or branched C₃-C₅ alkyl group, each optionally combined with D₁-D₁₀ deuteron, and/or hydroxy and/or carbonyl,
in subclass 1b, the substituent R8′ consists of an F1-F13 fluorine substituted C3-C7 alkenyl group, optionally combined with D1-D12 deuteron, and/or nitrile, and/or hydroxy and/or carbonyl, whereby the double bond being isolated from the Nitrogen,
in subclass 1c, the substituent R8′ consists of an F1-F11 fluorine substituted C3-C6 cycloalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in subclass 1d, the substituent R8′ consists of an F1-F17 fluorine substituted C3-C6 cycloalkylalkyl group, optionally combined with D1-D10 deuteron, and/or nitrile, and/or hydroxy, and/or carbonyl, and/or deuterated and nondeuterated C1-C3 alkyl and/or deuterated and nondeuterated C1-C3 alkenyl,
in subclass 1e, the substituent R8′ consists of an F1-F11 fluorine substituted C3-C7 alkynyl group, optionally combined with D1-D12 deuteron, and/or nitrile, and/or hydroxy and/or carbonyl, with the triple bond isolated from the amide Nitrogen,
in subclass 1f, the substituent R8′ consists of an F0-F7 fluorine substituted C2-C4 alkenyl group attached to the Nitrogen with the unsaturated part, yielding enamides, optionally combined with D1-D7 deuteron, and/or nitrile, and/or hydroxy and/or carbonyl,
in subclass 1g, the substituent R8′ consists of an F1-F5 fluorine substituted C2-C4 alkylalkynyl group attached to the Nitrogen with the unsaturated part, yielding ynamides, optionally combined with D1-D4 deuteron, and/or nitrile, and/or hydroxy and/or carbonyl,
in class 1h, being a subclass of class 1, the substituent R8′ consists of an F1-F13 fluorine substituted C1-3-O—C1-3 alkoxyalkyl group, optionally combined with D1-D12 deuteron, and/or nitrile, and/or hydroxy and/or carbonyl,
in subclass 1i, the substituent R8′ consists of an F0-F7 fluorine substituted C1-C3 alkoxy or C3-C4 cycloalkoxy group, each optionally combined with D1-D7 deuteron, and/or nitrile, and/or hydroxy and/or carbonyl,
in subclass 1j, the substituent R8′ consists of a nitrile attached to a C1-C3 alkyl group, optionally combined with F1-F7 fluorine, and/or D1-D7 deuteron, and/or hydroxy and/or carbonyl,
in subclass 1k, the substituent R8 consists of any D1-D6 deuteron combined with F1-F6 fluorine containing C1-C3 alkyl group optionally combined with hydroxy and/or carbonyl, and R8′ consists of a Hydrogen, a C1-C6 alkyl or a C3-C5 cycloalkyl or a C4-C7 cycloalkylalkyl group, optionally combined with hydroxy and/or carbonyl,
in subclass 1l, the substituent R8 consists of a D1-D7 deuteron or an F1-F7 fluorine, or of any D1-D6 deuteron combined with F1-F6 fluorine containing C1-C3 alkyl group optionally combined with hydroxy and/or carbonyl, and R8′ consists of a C2-C8 alkenyl or a C2-C8 alkynyl group, optionally combined with nitrile, and/or hydroxy and/or carbonyl,
in subclass 1m, the substituent R8 and R8′ are connected to each other to build an azacycloalkane with the amide Nitrogen, and are consisting of a D1-D10 deuteron or an F1-F10 fluorine, or of any D1-D9 deuteron combined with F1-F9 fluorine containing C3-C6 alkylene group optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group,
in subclass 1n, the substituent R8 and R8′ are connected to each other to build an azacycloalkane with the amide Nitrogen and are consisting of a C3-C6 alkylene group having a nondeuterated or deuterated C1-C3 alkenyl and/or a nondeuterated or deuterated C2-C3 alkynyl group attached, the azacycloalkane forming alkylene group further and optionally combined with nitrile, and/or hydroxy, and/or carbonyl and/or deuterated and nondeuterated C1-C3 alkyl, and/or deuterated and nondeuterated C1-C3 alkenyl and/or deuterated and nondeuterated C2-C3 alkynyl group;
class 2 is a lysergic acid amide as represented in FIGS. 4A-4G and FIGS. 5A-5C, further characterized in that it consists of 6-substituted 6-Nor-lysergic acid diethylamides, wherein R6 is consisting of substituents shown in subclasses, named subclasses 2a to 2i, whereby R6 is characterized as follows:
in subclass 2a, the substituent R6 consists of an F₁-F₁₁ fluorine substituted C₁-C₅ alkyl or branched C₃-C₅ alkyl group, each optionally combined with D₁-D₁₀ deuteron, and/or nitrile, and/or hydroxy and/or carbonyl,
in subclass 2b, being a subclass of class 2, the substituent R6 consists of an F1-F13 fluorine substituted C3-C7 alkenyl group, optionally combined with D1-D12 deuteron, and/or nitrile, and/or hydroxy and/or carbonyl, with the alkenyl double bond being isolated from Nitrogen,
in subclass 2c, the substituent R6 consists of an F1-F11 fluorine substituted C3-C7 alkynyl group with the triple bond isolated from N6 Nitrogen, optionally combined with D1-D10 deuteron, and/or nitrile, and/or hydroxy and/or carbonyl. In case the substituent R6 contains at least one nitrile, one hydroxy or one carbonyl group, R6 can also consist of a C3-C7 alkynyl group with the triple bond isolated from N6 Nitrogen, optionally combined with D1-D10 deuteron, and/or nitrile, and/or hydroxy and/or carbonyl,
in subclass 2d, the substituent R6 consists of a C3-C6 cycloalkyl group, optionally combined with F1-F11 fluorine, and/or D1-D11 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in subclass 2e, the substituent R6 consists of an F1-F17 fluorine substituted C4-C9 cycloalkylalkyl group, optionally combined with D1-D16 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl. In case the substituent R6 is not cyclopropylmethyl attached by the exocyclic methylene unit to the N6 Nitrogen of the ergoline structure, or it is cyclopropylmethyl attached by the exocyclic methylene unit to the N6 Nitrogen of the ergoline structure and contains at least one nitrile, one hydroxy or one carbonyl group, R6 can also consist of a C4-C9 cycloalkylalkyl group, optionally combined with D1-D17 deuteron, and/or nitrile, and/or hydroxy, and/or carbonyl, and/or deuterated and nondeuterated C1-C3 alkyl and/or deuterated and nondeuterated C1-C3 alkenyl and/or deuterated and nondeuterated C1-C3 alkynyl group,
in subclass 2f, the substituent R6 consists of an F0-F7 fluorine substituted C2-C4 alkenyl group attached to the Nitrogen with the unsaturated part, yielding enamines, optionally combined with D1-D7 deuteron, and/or nitrile, and/or hydroxy and/or carbonyl,
in subclass 2g, the substituent R6 consists of a C3-C6 oxacycloalkyl, a C3-C9 oxacycloalkylalkyl, a C3-C6 thiacycloalkyl or of a C3-C9 thiacycloalkylalkyl group, each optionally combined with F1-F19 fluorine, and/or D1-D19 deuteron, and/or nitrile, and/or hydroxy, and/or carbonyl, and/or deuterated and nondeuterated C1-C3 alkyl, and/or deuterated and nondeuterated C1-C3 alkenyl and/or deuterated and nondeuterated C2-C3 alkynyl,
in subclass 2h, the substituent R6 consists of an F0-F11 fluorine substituted C1-C5 alkoxy or C3-C6 cycloalkoxy or C2-C6 alkenoxy group, each optionally combined with D1-D11 deuteron, and/or nitrile, and/or hydroxy and/or carbonyl,
in subclass 2i, the substituent R6 consists of an aryl, a heteroaryl, an arylmethyl or a heteroarylmethyl group, each optionally combined with F0-F7 fluorine, and/or D1-D7 deuteron, and/or one or more of the following substituents that themselves can optionally be fluorinated and/or deuterated: halogen, nitrile, nitro, hydroxy, carbonyl, C1-C4 alkoxy, C1-C4 alkyl, C1-C4 alkenyl, C1-C4 alkynyl, C3-C6 cycloalkyl, C3-C6 cycloalkoxy, C3-C6 alkenoxy, C3-C6 alkynoxy, methylenedioxy, C1-C4 alkylthio, C3-C6 alkenylthio, C3-C6 alkynylthio, C3-C6 cycloalkylthio, and the R6 substituent, as defined for class 2i above, can further be annulated;
class 3 is a lysergic acid amide as represented in FIG. 6A, further characterized in that it consists of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C);
class 4 is a lysergic acid amide as represented in FIG. 6B, further characterized in that it consists of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group:
a) any acyl;
b) unsubstituted and substituted carbamoyl;
c) amide-bound amino acid;
d) alkyl, alkenyl or alkynyl;
e) alkoxy, alkenoxy or alkynoxy;
f) any of the substituents described under a) to e), substituted with one or more fluorine atoms;
g) any of the substituents described under a) to e), substituted with one or more deuteron atoms;
h) any of the substituents described under a) to e), substituted with one or more fluorine atoms and one or more deuteron atoms; and
class 5 (FIG. 6C), consisting of a monodeuterated up to a fully deuterated ergoline core structure, and additionally consisting of any possible combination of the substituents R8 and R8′ from class 1 and its subclasses 1a to 1n (FIG. 2A to FIG. 3H) with the substituents R6 from class 2 and its subclasses 2a to 2i (FIG. 4A to FIG. 5C) and with combination of an N1 Nitrogen substituent on the ergoline substructure from the following group: a) Hydrogen; b) any acyl; c) unsubstituted and substituted carbamoyl; d) amide-bound amino acid; e) alkyl, alkenyl or alkynyl; f) alkoxy, alkenoxy or alkynoxy; g) any of the substituents described under a) to f), substituted with one or more fluorine atoms; h) any of the substituents described under a) to f), substituted with one or more deuteron atoms; i) any of the substituents described under a) to f), substituted with one or more fluorine atoms and one or more deuteron atoms;
increasing serotonin 5-HT2A and 5-HT2C receptor interaction in the mammal; and
inducing psychoactive effects.

8. The method of claim 7, wherein the compound is chosen from the group consisting of a racemate, a single enantiomer, a diastereomer, or a mixture of enantiomers or diastereomers or epimers in any ratio, a single and a mixture E- or Z-configurational isomer in any ratio, a single and a mixture cis or trans configurational isomer in any ratio and any combination thereof.

9. The method of claim 7, wherein the psychoactive effects include psychedelic or empathogenic effects having intensity, effect quality, or duration of effect in a mammal similar or different in comparison to that of LSD.

10. The method of claim 7, wherein the compound is administered to mammals for substance-assisted psychotherapy.

11. The method of claim 7, wherein the compound is administered to allow for changing dose potency in comparison to LSD.

12. The method of claim 7, wherein the compound is administered to allow for tailoring and treatment individualization to the mammal's therapeutic need.

13. The method of claim 7, wherein the mammal is a human.

14. A method of treating an individual, including the steps of:
administering a pharmaceutically effective amount of a compound of FIG. 1A to the individual; and
treating the individual.

15. The pharmacologically active compound of claim 1, wherein said compound is chosen from the group consisting of compound 2l, compound 12c, compound 16a, and compound 16b and further characterized in that the compound is metabolized faster than LSD resulting in a shorter duration of acute action.

16. The pharmacologically active compound of claim 1, wherein said compound is chosen from the group consisting of compounds 2a-2n, compounds 12a-12g, compound 13, compounds 14a-c, and compounds 16a-c and further characterized in that the compound has a similar potency to LSD resulting in similar small doses being psychoactive and therapeutically active.
